(19) United States
(12) Patent Application Publication
(10) Pub. No.: US 2004/0039208 A1
(43) Pub. Date: Feb. 26, 2004

(54) Process for making n-aryl-anthranilic acids and their derivatives

(76) Inventors: Michael Huai Gu Chen, Ann Arbor, MI (US); Edward Mark Davis, Zeeland, MI (US); Javier Magano, Ypsilanti, MI (US); Thomas Norman Nanninga, Holland, MI (US); Derick Dale Winkle, Zeeland, MI (US)

Correspondence Address: Suzanne M Harvey, Warner Lambert Company, 2800 Plymouth Road, Ann Arbor, MI, 48105, US

(21) Appl. No.: 10/344,294

(22) PCT Filed: Jul. 20, 2001
(86) PCT No.: PCT/US01/22948

(51) Int. Cl. C07D 213/46; C07C 255/50; C07C 211/54

(52) U.S. Cl. 546/328; 548/577; 558/408; 564/433; 564/434

(57) ABSTRACT

The present invention relates to a process for the preparation of N-arylanthranilic acids, and a process for the preparation of N-aryl anthranilic esters, amides, and hydroxamic esters.

This application is a 371 application of PCT/US01/22948 filed Jul. 20, 2001, which claims the benefit of priority to U.S. provisional application Serial No. 60/228,206 filed Aug. 25, 2000.

### BACKGROUND OF THE INVENTION

The present invention relates to a process for the preparation of N-aryl anthranilic acids, which are useful pharmaceutical agents and, for example, are known anti-inflammatory agents. In addition, N-aryl-anthranilic acids can serve as intermediates in the preparation of N-aryl anthranilic amides, N-aryl-anthranilic hydroxamic acids, and N-aryl anthranilic hydroxamic acid esters. Certain N-aryl anthranilic hydroxamic acids and N-aryl anthranilic hydroxamic acid esters inhibit certain dual specificity kinase enzymes involved in proliferative diseases such as cancer and restenosis.

Proliferative diseases are caused by a defect in the intracellular signaling system, or the signal transduction mechanism of certain proteins. Cancer, for example, is commonly caused by a series of defects in these signaling proteins, resulting from a change either in their intrinsic activity or in their cellular concentrations. For example, a cell may produce a growth factor that binds to its own receptors, resulting in an autocrine loop, which continually stimulates proliferation. Mutations or over expression of intracellular signaling proteins such as Ras can lead to spurious mitogenic signals within a cell. Some of the most common mutations occur in genes encoding for Ras, which is a G-protein that is in an activated state when it is bound to GTP, and in an inactivated state when it is bound to GDP. Activation and inactivation of Ras is regulated in normal cells.

The above mentioned growth factor receptors, and many other mitogenic receptors, when activated, lead to Ras being converted from the GDP-bound state to the GTP-bound state. This signal is thought to be an absolute prerequisite for proliferation in most cell types. Defects in this signaling system, especially in the deactivation of the Ras·GTP complex, are common in cancers, and lead to the signaling cascade downstream from Ras being chronically activated.

Activated Ras leads in turn to the activation of a cascade of serine/threonine kinases. One of the groups of kinases known to require an active Ras-GTP for its own activation is the Raf family of kinases. The Raf kinases in turn activate mitogen-activated protein kinase (“MAP kinase” or “MAPK”)/extracellular signal-regulated kinase (“ERK”), also known as “MAP/ERK kinase,” (“MEK”), which then activates one of at least three known MAP kinases, namely ERK. Activation of MAP kinase by mitogens appears to be essential for proliferation, and constitutive activation of this kinase is thought to be sufficient to induce cellular transformation. Blockade of downstream Ras signaling, for example by use of a dominant negative Raf-1 protein, can completely inhibit mitogenesis, whether induced from cell surface receptors or from oncogenic Ras mutants.

Accordingly, although Ras is not itself a protein kinase, it participates in the activation of Raf and other kinases. This participation most likely occurs through a phosphorylation mechanism. For example, once activated, Raf and other kinases are known to phosphorylate MEK on two closely adjacent serine residues, namely S²¹⁸ and S²²² in the case of MEK-1, which is a prerequisite for activation of MEK as a kinase. Phosphorylated MEK in turn phosphorylates MAP kinase on tyrosine, Y¹⁸⁵, and threonine, T¹⁸³. This double phosphorylation activates MAP kinase at least 100-fold, and leads to the activated MAP kinase catalyzing the phosphorylation of a large number of proteins, including several transcription factors and other kinases. Many of these MAP kinase phosphorylations are mitogenically activating for the target protein, whether the target protein is another kinase, a transcription factor, or other cellular protein.

MEK is also activated by several kinases other than Raf-1, including MEK itself, which appears to be a signal integrating kinase. As far as is currently known, MEK is highly specific for the phosphorylation of MAP kinase. In fact, no substrate for MEK other than MAP kinase has been demonstrated to date, and MEK does not phosphorylate peptides based on the MAP kinase phosphorylation sequence, or even phosphorylate denatured MAP kinase. MEK also appears to associate strongly with MAP kinase prior to phosphorylating it, suggesting that phosphorylation of MAP kinase by MEK may require a prior strong interaction between the two proteins. Accordingly, it is thought that selective inhibitors of MEK, possibly operating through allosteric mechanisms rather than through the usual blockade of the ATP binding site, may be valuable.

This invention provides processes for making compounds that are highly specific inhibitors of the kinase activity of MEK. Both in enzyme assays and in whole cells, the compounds made by the processes of this invention inhibit the phosphorylation of MAP kinase by MEK, thus preventing the activation of MAP kinase in cells in which the Ras cascade has been activated. One result of this enzyme inhibition is a reversal of transformed phenotype of some cells types, as measured both by the ability of the transformed cells to grow in an anchorage-independent manner and by the ability of some transformed cell lines to proliferate independently of external mitogens.

Thus, a process of synthesizing N-aryl anthranilic acids that results in higher yields and minimizes the amount of required aniline, often an expensive, hard to synthesize, or hard to remove (unreacted or excess aniline from the reaction mixture after reaction is complete) starting material, would be desirable. Such a process would allow successful commercial scale production of the N-aryl anthranilic acids.

The present invention unexpectedly provides high-yielding processes for preparing N-aryl anthranilic acids and derivatives thereof comprising coupling about 1 mole equivalent of an aniline with about 1 mole equivalent of an ortho-halobenzoic acid. For example, contrary to the law of mass action, the yield of product provided by the present process is unexpectedly higher than the yield of product provided by a process that employs 2 mol equivalents of the aniline. Further, the invention process allows successful commercial scale production of the N-aryl anthranilic acids and derivatives thereof. These and other advantages of this invention will be more fully described in the following text.

### SUMMARY OF THE INVENTION

One embodiment of the present invention is a process, hereinafter referred to as Process Embodiment 1, of synthesizing a compound of Formula I

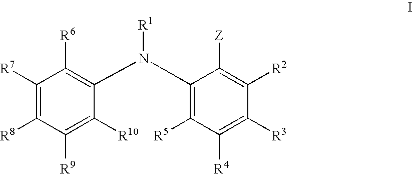

or a pharmaceutically acceptable salt thereof, wherein:

R¹ is hydrogen, alkyl, alkoxy, or aryl;

R², R³, R⁴, R⁵, R⁶, R⁷, R⁸, R⁹, and R¹⁰ are each independently selected from:

hydrogen,

halo,

alkyl,

aryl,

a heterocyclic group,

haloalkyl,

alkoxy,

nitro,

CN,

—(O)ₘ—(CH₂)ₙ—R¹¹, or

—[N(H)]ₘ—(CH₂)ₙ—R¹¹, wherein m, n, and R¹¹ are as defined below,

or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms,

or R¹ and R⁶ may be taken together with the nitrogen atom to which R¹ is attached, the carbon atom to which R⁶ is attached, and the carbon atom contiguous to said nitrogen atom to which R¹ is attached and said carbon atom to which R⁶ is attached, to form a 5-membered or 6-membered, aromatic or dihydro-aromatic ring having carbon atoms and 1 or 2 nitrogen atoms;

R¹¹ is hydrogen, hydroxy, —CO₂H, or N(R¹²)R¹³,

R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;

m is an integer of 0 or 1;

n is an integer selected from 0, 1, 2, 3, 4; and

Z is COOH, COOM, COOR¹⁵, —C(O)R¹⁵, —C(O)N(R¹⁶)R¹⁷, —C(O)N(R¹⁸)OR¹⁹, NO₂, or CN, wherein

M is a Group I metal cation or a hemi Group II metal cation,

R¹⁵ is alkyl, alkenyl, alkynyl, aryl, or a heterocyclic group, and

R¹⁶, R¹⁷, R¹⁸, and R¹⁹ are each independently selected from hydrogen, alkyl, alkenyl, phenyl, and benzyl, or

R¹⁶ and R ¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;

comprising reacting a compound of Formula (A)

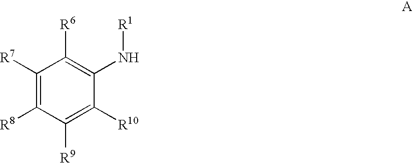

wherein R¹, R⁶, R⁷, R⁸, R⁹, and R¹⁰ are as defined above, with a compound of Formula (B)

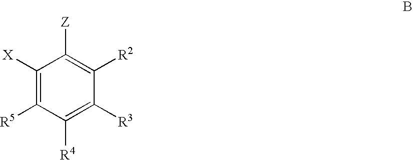

wherein Z, R², R³, R⁴, and R⁵ are as defined above, and X is halo or O-LG,

wherein LG is SO₂R²⁰ or P(═O)(OR²⁰)₂, wherein R²⁰ is alkyl or aryl, optionally in a solvent, and in the presence of from about 1 mol equivalent to about 10 mol equivalents of a base, wherein the base is selected from:

a Group I metal cation hydride or a Group 2 metal cation hydride, including lithium hydride, sodium hydride, potassium hydride, and calcium hydride,

a Group I metal cation dialkylamide or a Group 2 metal cation dialkylamide, including lithium diisopropylamide,

a Group I metal cation amide or a Group 2 metal cation amide, including lithium amide, sodium amide, potassium amide, and

a Group I metal cation alkoxide or a Group 2 metal cation alkoxide, including sodium ethoxide, potassium tert-butoxide, and magnesium ethoxide, for a time, and at a temperature, sufficient to yield a compound of Formula I.

Another embodiment of the present invention is a process of Process Embodiment 1, wherein the base is selected from: lithium diisopropylamide, lithium hydride, sodium hydride, potassium hydride, lithium amide, sodium amide, potassium amide, sodium methoxide, sodium ethoxide, and potassium tert-butoxide.

Another embodiment of the present invention is a process of Process Embodiment 1, wherein the base is selected from lithium hydride, sodium hydride, and potassium hydride.

Another embodiment of the present invention is a process of Process Embodiment 1, wherein the base is lithium hydride.

Another embodiment of the present invention is a process of Process Embodiment 1, wherein the base is selected from lithium amide, sodium amide, and potassium amide.

Another embodiment of the present invention is a process of Process Embodiment 1, wherein the base is lithium amide.

Another embodiment of the present invention is a process of Process Embodiment 1, wherein the base is lithium diisopropylamide.

Another embodiment of the present invention is a process of Process Embodiment 1, wherein the base is selected from sodium methoxide, sodium ethoxide, and potassium tert-butoxide.

Another embodiment of the present invention is a process of Process Embodiment 1, wherein from 1 to 5 mol equivalents of base are employed initially, and optionally from 0.5 to 4 additional mol equivalents of base are added to the reaction after a time, wherein said 0.5 to 4 additional mol equivalents of base are added in one portion or are added sequentially in unequal or equal portions at unequal or equal time intervals.

Another embodiment of the present invention is a process of Process Embodiment 1, wherein said 0.5 to 4 additional mol equivalents of base are added sequentially to the reaction in unequal portions of decreasing size.

Another embodiment of the present invention is a process of Process Embodiment 1, wherein in the compound of formula (B), Z is COOH and 2 mol equivalents of base are employed initially or Z is COOM and 1 mol equivalent of base is employed initially, and said 0.5 to 4 additional mol equivalents of base are added sequentially to the reaction in unequal portions of decreasing size as follows: about 0.5 mol equivalents, followed by about 0.25 mol equivalents, followed by about 0.13 mol equivalents, followed by about 0.06 mol equivalents, optionally followed by about 0.03 mol equivalents, followed by about 0.015 mol equivalents.

Another embodiment of the present invention is a process of Process Embodiment 1, wherein R¹ is hydrogen.

Another embodiment of the present invention is a process of Process Embodiment 1, wherein X is fluoro.

Another embodiment of the present invention is a process of Process Embodiment 1, wherein X is O-LG, wherein LG is SO₂CF₃ or P(═O)(OCH₂CH₃)₂.

Another embodiment of the present invention is a process of Process Embodiment 1, wherein X is O-LG, wherein LG is SO₂CF₃ or P(═O)(OCH₂CH₃)₂, further comprising an organopalladium catalyst.

Another embodiment of the present invention is a process of Process Embodiment 1, wherein R², R³, R⁴, and R⁵ are each independently selected from hydrogen, alkoxy, fluoro, chloro, bromo, and iodo.

Another embodiment of the present invention is a process of Process Embodiment 1, wherein R⁶, R⁷, R⁸, R⁹, and R¹⁰ are each independently selected from hydrogen, alkyl, fluoro, chloro, bromo, and iodo.

Another embodiment of the present invention is a process of Process Embodiment 1, wherein Z is COOH or COOM.

Another embodiment of the present invention is a process of Process Embodiment 1, wherein R¹ is hydrogen, X is fluoro, R², R³, R⁴, and R⁵ are each independently selected from hydrogen, alkoxy, fluoro, chloro, bromo, and iodo, R⁶, R⁷, R⁸, R⁹, and R¹⁰ are each independently selected from hydrogen, methyl, fluoro, chloro, bromo, and iodo, and Z is COOH or COOM.

Another embodiment of the present invention is a process of Process Embodiment 1, wherein a solvent is present and the solvent comprises acetonitrile, tetrahydrofuran, 1,2-diethoxyethane, 2,2-dimethoxypropane, 1,2-dimethoxypropane, diethylether, dioxane, or methyl tert-butylether.

Another embodiment of the present invention is a process of Process Embodiment 1, wherein a solvent is present and the solvent comprises tetrahydrofuran or acetonitrile.

Another embodiment of the present invention is a process of Process Embodiment 1, wherein a solvent is present and the solvent comprises a mixture of from about 1 part by volume of acetonitrile and about 1 part by volume of tetrahydrofuran to about 5 parts by volume of acetonitrile and about 1 part by volume of tetrahydrofuran.

Another embodiment of the present invention is a process of Process Embodiment 1, wherein when the base is added, the reaction mixture is at a temperature of from −78° C. to 150° C.

Another embodiment of the present invention is a process of Process Embodiment 1, or any one of the other above embodiments of a process of Process Embodiment 1, wherein the compound of Formula I is a compound of formula

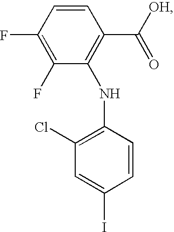

or a pharmaceutically acceptable salt thereof.

Another embodiment of the present invention is a process of Process Embodiment 1, or any one of the other above embodiments of a process of Process Embodiment 1 except the one immediately above, wherein the compound of Formula I is a compound of formula

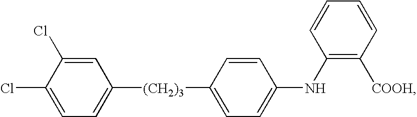

or a pharmaceutically acceptable salt thereof.

Another embodiment of the present invention is a process of Process Embodiment 1, wherein the compound of Formula I is a compound of Formula Ia

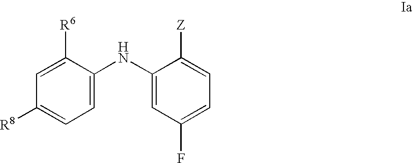

or a pharmaceutically acceptable salt thereof,

wherein R⁶ is halo or methyl, R⁸ is bromo or iodo, and Z is COOH, COOM, COOR¹⁵, —C(O)R¹⁵, —C(O)N(R¹⁶)R¹⁷, —C(O)N(R¹⁸)OR¹⁹, NO₂, or CN, wherein

M is a Group I metal cation or a hemi Group II metal cation,

R¹⁵ is alkyl, alkenyl, alkynyl, aryl, or a heterocyclic group, and

R¹⁶, R¹⁷, R¹⁸, and R¹⁹ are each independently selected from hydrogen, alkyl, alkenyl, phenyl, and benzyl, or

R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein

R¹⁴ is hydrogen or alkyl.

Another embodiment of the present invention is a process of Process Embodiment 1, wherein the compound of Formula I is a compound of Formula Ib

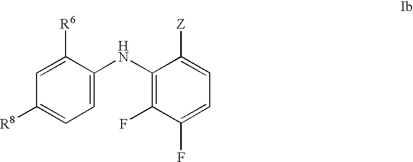

or a pharmaceutically acceptable salt thereof,

wherein R⁶ is halo or methyl, R⁸ is bromo or iodo, and Z is COOH, COOM, COOR¹⁵, —C(O)R¹⁵, —C(O)N(R¹⁶)R¹⁷, —C(O)N(R¹⁸)OR¹⁹, NO₂, or CN, wherein

M is a Group I metal cation or a hemi Group II metal cation,

R¹⁵ is alkyl, alkenyl, alkynyl, aryl, or a heterocyclic group, and

R¹⁶, R¹⁷, R¹⁸, and R¹⁹ are each independently selected from hydrogen, alkyl, alkenyl, phenyl, and benzyl, or

R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein

R¹⁴ is hydrogen or alkyl.

Another embodiment of the present invention is a process of Process Embodiment 1, wherein the compound of Formula I is a compound of Formula Ic1

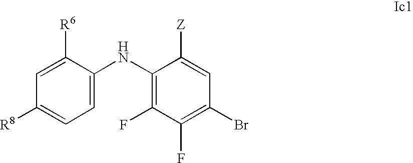

or a pharmaceutically acceptable salt thereof,

wherein R⁶ is halo or methyl, R⁸ is bromo or iodo, and Z is COOH, COOM, COOR¹⁵, —C(O)R¹⁵, —C(O)N(R¹⁶)R¹⁷, —C(O)N(R¹⁸)OR¹⁹, NO₂, or CN, wherein

M is a Group I metal cation or a hemi Group II metal cation,

R¹⁵ is alkyl, alkenyl, alkynyl, aryl, or a heterocyclic group, and

R¹⁶, R¹⁷, R¹⁸, and R¹⁹ are each independently selected from hydrogen, alkyl, alkenyl, phenyl, and benzyl, or

R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein

R¹⁴ is hydrogen or alkyl.

Another embodiment of the present invention is a process of Process Embodiment 1, wherein the compound of Formula I is a compound of Formula Ic2

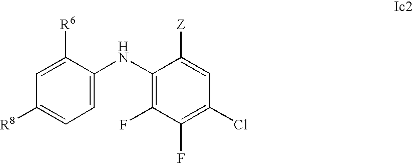

or a pharmaceutically acceptable salt thereof,

wherein R⁶ is halo or methyl, R⁸ is bromo or iodo, and Z is COOH, COOM, COOR¹⁵, —C(O)R¹⁵, —C(O)N(R¹⁶)R¹⁷, —C(O)N(R¹⁸)OR¹⁹, NO₂, or CN, wherein

M is a Group I metal cation or a hemi Group II metal cation,

R¹⁵ is alkyl, alkenyl, alkynyl, aryl, or a heterocyclic group, and

R¹⁶, R¹⁷, R¹⁸, and R¹⁹ are each independently selected from hydrogen, alkyl, alkenyl, phenyl, and benzyl, or

R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein

R¹⁴ is hydrogen or alkyl.

Another embodiment of the present invention is a process of Process Embodiment 1, wherein the compound of Formula I is a compound of Formula Id

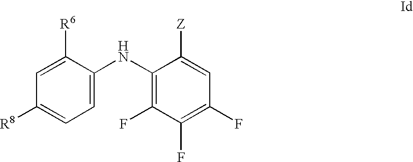

or a pharmaceutically acceptable salt thereof,

wherein R⁶ is halo or methyl, R⁸ is bromo or iodo, and Z is COOH, COOM, COOR¹⁵, —C(O)R¹⁵, —C(O)N(R¹⁶)R¹⁷, —C(O)N(R¹⁸)OR¹⁹, NO₂, or CN, wherein

M is a Group I metal cation or a hemi Group II metal cation,

R¹⁵ is alkyl, alkenyl, alkynyl, aryl, or a heterocyclic group, and

R¹⁶, R¹⁷, R¹⁸, and R¹⁹ are each independently selected from hydrogen, alkyl, alkenyl, phenyl, and benzyl, or

R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein

R¹⁴ is hydrogen or alkyl.

Another embodiment of the present invention is a process of Process Embodiment 1, wherein the compound of Formula I is a compound of formula

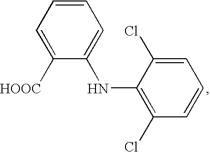

or a pharmaceutically acceptable salt thereof.

Another embodiment of the present invention is a process of Process Embodiment 1, wherein the compound of Formula I is a compound of formula

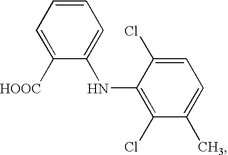

or a pharmaceutically acceptable salt thereof.

Another embodiment of the present invention is a process of Process Embodiment 1, wherein the compound of Formula I is a compound of formula

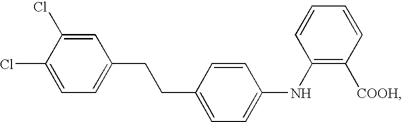

or a pharmaceutically acceptable salt thereof.

Another embodiment of the present invention is a process of Process Embodiment 1, wherein the compound of Formula I is a compound of formula

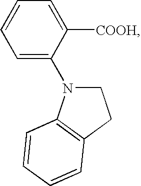

or a pharmaceutically acceptable salt thereof.

Another embodiment of the present invention is a process of Process Embodiment 1, wherein the compound of Formula I is a compound of formula

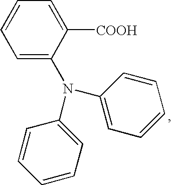

or a pharmaceutically acceptable salt thereof.

Another embodiment of the present invention is a process of Process Embodiment 1, wherein the compound of Formula I is a compound of formula

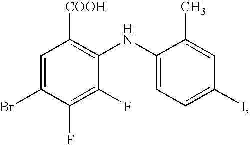

or a pharmaceutically acceptable salt thereof.

Another embodiment of the present invention is a process of Process Embodiment 1, wherein the compound of Formula I is a compound of formula

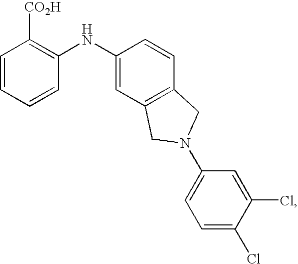

or a pharmaceutically acceptable salt thereof.

Another embodiment of the present invention is a process of Process Embodiment 1, wherein the compound of Formula I is a compound of formula

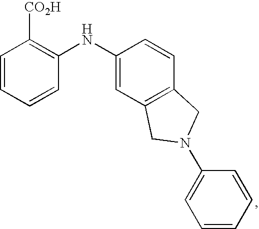

or a pharmaceutically acceptable salt thereof.

Another embodiment of the present invention is a process, hereinafter referred to as Process Embodiment 1A, of synthesizing a compound of Formula I

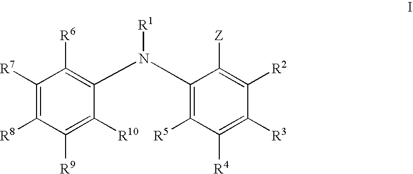

or a pharmaceutically acceptable salt thereof,

wherein:

R¹ is hydrogen, alkyl, alkoxy, or aryl;

R², R³, R⁴, R⁵, R⁶, R⁷, R⁸, R⁹, and R¹⁰ are each independently selected from:

hydrogen,

halo,

alkyl,

aryl,

a heterocyclic group,

haloalkyl,

alkoxy,

nitro,

CN,

—(O)ₘ—(CH₂)ₙ—R¹¹, or

—[N(H)]ₘ—(CH₂)ₙ—R¹¹, wherein m, n, and R¹¹ are as defined below,

or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms,

or R¹ and R⁶ may be taken together with the nitrogen atom to which R¹ is attached, the carbon atom to which R⁶ is attached, and the carbon atom contiguous to said nitrogen atom to which R¹ is attached and said carbon atom to which R⁶ is attached, to form a 5-membered or 6-membered, aromatic or dihydro-aromatic ring having carbon atoms and 1 or 2 nitrogen atoms;

R¹¹ is hydrogen, hydroxy, —CO₂H, or N(R¹²)R¹³,

R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;

m is an integer of 0 or 1;

n is an integer selected from 0, 1, 2, 3, 4; and

Z is COOH, COOM, COOR¹⁵, —C(O)R¹⁵, —C(O)N(R¹⁶)R¹⁷,

—C(O)N(R^{1 8})OR¹⁹, NO₂, or CN, wherein M is a Group I, or a hemi Group II, metal cation, R¹⁵ is alkyl, alkenyl, alkynyl, aryl, or a heterocyclic group, and R¹⁶, R¹⁷, R¹⁸, and R¹⁹ are each independently selected from hydrogen, alkyl, alkenyl, phenyl, and benzyl, or

R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;

comprising reacting a compound of Formula (A)

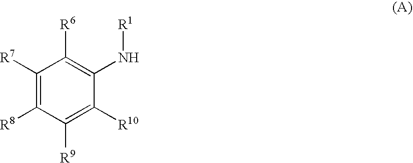

wherein R¹, R⁶, R⁷, R⁸, R⁹, and R¹⁰ are as defined above, with a compound of Formula (B)

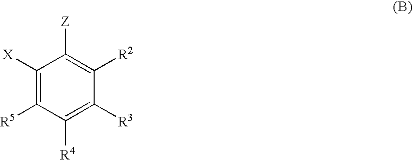

wherein Z is COOR¹⁵, —C(O)R¹⁵, —C(O)N(R¹⁶)R¹⁷, —C(O)N(R¹⁸)OR¹⁹, NO₂, or CN, and R²—R⁵ and R¹⁵—R¹⁹ are as defined above, and X is halo or O-LG, wherein LG is SO₂R²⁰ or P(═O)(OR²⁰)₂, wherein R²⁰ is alkyl or aryl, optionally in a solvent, and in the presence of from about 1 mol equivalent to about 10 mol equivalents of a base, wherein the base is selected from:

a Group I metal cation bis(trialkylsilyl)amide or a Group 2 metal cation bis(trialkylsilyl)amide, including lithium bis(trimethylsilyl)amide, sodium bis(trimethylsilyl)amide, or potassium bis(trimethylsilyl)amide, for a time, and at a temperature, sufficient to yield a compound of Formula I.

Another embodiment of the present invention is a process of Process Embodiment 1A, wherein the base is selected from: lithium bis(trimethylsilyl)amide, sodium bis(trimethylsilyl)amide, and potassium bis(trimethylsilyl)amide.

Another embodiment of the present invention is a process of Process Embodiment 1A, wherein the base is lithium bis(trimethylsilyl)amide.

Another embodiment of the present invention is a process of Process Embodiment 1A, wherein from 1 to 5 mol equivalents of base are employed initially, and optionally from 0.5 to 4 additional mol equivalents of base are added to the reaction after a time, wherein said 0.5 to 4 additional mol equivalents of base are added in one portion or are added sequentially in unequal or equal portions at unequal or equal time intervals.

Another embodiment of the present invention is a process of Process Embodiment 1A, wherein said 0.5 to 4 additional mol equivalents of base are added sequentially to the reaction in unequal portions of decreasing size.

Another embodiment of the present invention is a process of Process Embodiment 1A, wherein R¹ is hydrogen.

Another embodiment of the present invention is a process of Process Embodiment 1A, wherein X is fluoro.

Another embodiment of the present invention is a process of Process Embodiment 1A, wherein X is O-LG, wherein LG is SO₂CF₃ or P(═O)(OCH₂CH₃)₂.

Another embodiment of the present invention is a process of Process Embodiment 1A, wherein X is O-LG, wherein LG is SO₂CF₃ or P(═O)(OCH₂CH₃)₂, further comprising an organopalladium catalyst.

Another embodiment of the present invention is a process of Process Embodiment 1A, wherein R², R³, R⁴, and R⁵ are each independently selected from hydrogen, alkoxy, fluoro, chloro, bromo, and iodo.

Another embodiment of the present invention is a process of Process Embodiment 1A, wherein R⁶, R⁷, R⁸, R⁹, and R¹⁰ are each independently selected from hydrogen, alkyl, fluoro, chloro, bromo, and iodo.

Another embodiment of the present invention is a process of Process Embodiment 1A, wherein Z is —C(O)N(R¹⁸)OR¹⁹, wherein R¹⁸ and R¹⁹ are each independently selected from hydrogen, alkyl, alkenyl, phenyl, and benzyl.

Another embodiment of the present invention is a process of Process Embodiment 1A, wherein R¹ is hydrogen, X is fluoro, R², R³, R⁴, and R⁵ are each independently selected from hydrogen, alkoxy, fluoro, chloro, bromo, and iodo, R⁶, R⁷, R⁸, R⁹, and R¹⁰ are each independently selected from hydrogen, methyl, fluoro, chloro, bromo, and iodo, and Z is —C(O)N(R¹⁸)OR¹⁹, wherein R¹⁸ and R¹⁹ are each independently selected from hydrogen, alkyl, alkenyl, phenyl, and benzyl.

Another embodiment of the present invention is a process of Process Embodiment 1A, wherein a solvent is present and the solvent comprises acetonitrile, tetrahydrofuran, 1,2-diethoxyethane, 2,2-dimethoxypropane, 1,2-dimethoxypropane, diethylether, dioxane, or methyl tert-butylether.

Another embodiment of the present invention is a process of Process Embodiment 1A, wherein a solvent is present and the solvent comprises tetrahydrofuran or acetonitrile.

Another embodiment of the present invention is a process of Process Embodiment 1A, wherein a solvent is present and the solvent comprises a mixture of from about 1 part by volume of acetonitrile and about 1 part by volume of tetrahydrofuran to about 5 parts by volume of acetonitrile and about 1 part by volume of tetrahydrofuran.

Another embodiment of the present invention is a process of Process Embodiment 1A, wherein when the base is added, the reaction mixture is at a temperature of from −78° C. to 150° C.

Another embodiment of the present invention is a process of Process Embodiment 1A, or any one of the other above embodiments of a process of Process Embodiment 1A, wherein the compound of Formula I is a compound of formula

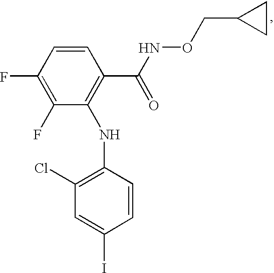

or a pharmaceutically acceptable salt thereof.

Another embodiment of the present invention is a process of Process Embodiment 1A, wherein the compound of Formula I is a compound of Formula Ia

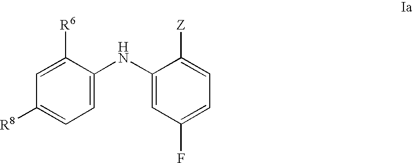

or a pharmaceutically acceptable salt thereof,

wherein R⁶ is halo or methyl, R⁸ is bromo or iodo, and Z is COOH, COOM, COOR¹⁵, —C(O)R¹⁵, —C(O)N(R¹⁶)R¹⁷, —C(O)N(R¹⁸)OR¹⁹, NO₂, or CN, wherein

M is a Group I metal cation or a hemi Group II metal cation,

R¹⁵ is alkyl, alkenyl, alkynyl, aryl, or a heterocyclic group, and

R¹⁶, R¹⁷, R¹⁸, and R¹⁹ are each independently selected from hydrogen, alkyl, alkenyl, phenyl, and benzyl, or

R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl.

Another embodiment of the present invention is a process of Process Embodiment 1A, wherein the compound of Formula I is a compound of Formula Ib

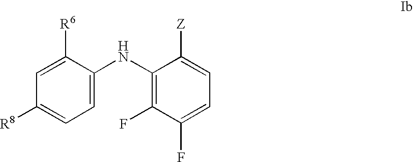

or a pharmaceutically acceptable salt thereof,

wherein R⁶ is halo or methyl, R⁸ is bromo or iodo, and Z is COOH, COOM, COOR¹⁵, —C(O)R¹⁵, —C(O)N(R¹⁶)R¹⁷, —C(O)N(R¹⁸)OR¹⁹, NO₂, or CN, wherein

M is a Group I metal cation or a hemi Group II metal cation,

R¹⁵ is alkyl, alkenyl, alkynyl, aryl, or a heterocyclic group, and

R¹⁶, R¹⁷, R¹⁸, and R¹⁹ are each independently selected from hydrogen, alkyl, alkenyl, phenyl, and benzyl, or

R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl.

Another embodiment of the present invention is a process of Process Embodiment 1A, wherein the compound of Formula I is a compound of Formula Ic1

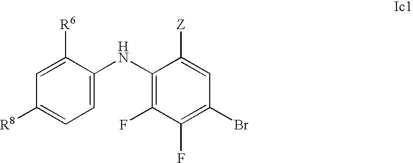

or a pharmaceutically acceptable salt thereof,

wherein R⁶ is halo or methyl, R⁸ is bromo or iodo, and Z is COOH, COOM, COOR¹⁵, —C(O)R¹⁵, —C(O)N(R¹⁶)R¹⁷, —C(O)N(R¹⁸)OR¹⁹, NO₂, or CN, wherein

M is a Group I metal cation or a hemi Group II metal cation,

R¹⁵ is alkyl, alkenyl, alkynyl, aryl, or a heterocyclic group, and

R¹⁶, R¹⁷, R¹⁸, and R¹⁹ are each independently selected from hydrogen, alkyl, alkenyl, phenyl, and benzyl, or

R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl.

Another embodiment of the present invention is a process of Process Embodiment 1A, wherein the compound of Formula I is a compound of Formula Ic2

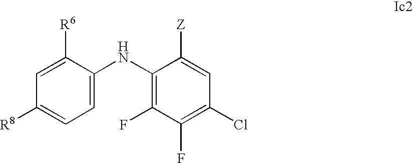

or a pharmaceutically acceptable salt thereof,

wherein R⁶ is halo or methyl, R⁸ is bromo or iodo, and Z is COOH, COOM, COOR¹⁵, —C(O)R¹⁵, —C(O)N(R¹⁶)R¹⁷, —C(O)N(R¹⁸)OR¹⁹, NO₂, or CN, wherein

M is a Group I metal cation or a hemi Group II metal cation,

R¹⁵ is alkyl, alkenyl, alkynyl, aryl, or a heterocyclic group, and

R¹⁶, R¹⁷, R¹⁸, and R¹⁹ are each independently selected from hydrogen, alkyl, alkenyl, phenyl, and benzyl, or

R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl.

Another embodiment of the present invention is a process of Process Embodiment 1A, wherein the compound of Formula I is a compound of Formula Id1

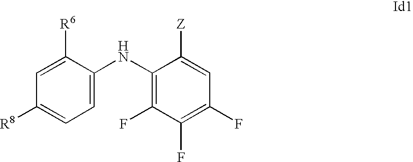

or a pharmaceutically acceptable salt thereof,

wherein R⁶ is halo or methyl, R⁸ is bromo or iodo, and Z is COOH, COOM, COOR¹⁵, —C(O)R¹⁵, —C(O)N(R¹⁶)R¹⁷, —C(O)N(R¹⁸)OR¹⁹, NO₂, or CN, wherein

M is a Group I metal cation or a hemi Group II metal cation,

R¹⁵ is alkyl, alkenyl, alkynyl, aryl, or a heterocyclic group, and

R¹⁶, R¹⁷, R¹⁸, and R¹⁹ are each independently selected from hydrogen, alkyl, alkenyl, phenyl, and benzyl, or

R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein

R¹⁴ is hydrogen or alkyl.

Another embodiment of the present invention is a process of Process Embodiment 1A, further comprising hydrolyzing the compound of Formula I wherein Z is COOR¹⁵, wherein R¹⁵ is alkyl, alkenyl, alkynyl, aryl, or a heterocyclic group, to provide the compound of Formula Id2

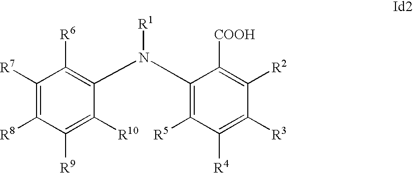

or a pharmaceutically acceptable salt thereof,

wherein:

R¹ is hydrogen, alkyl, alkoxy, or aryl;

R², R³, R⁴, R⁵, R⁶, R⁷, R⁸, R⁹, and R¹⁰ are each independently selected from:

hydrogen,

halo,

alkyl,

aryl,

a heterocyclic group,

haloalkyl,

alkoxy,

nitro,

CN,

—(O)ₘ—(CH₂)ₙ—R¹¹, or

—[N(H)]ₘ—(CH₂)ₙ—R¹¹, wherein m, n, and R¹¹ are as defined below,

or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms,

or R ¹ and R⁶ may be taken together with the nitrogen atom to which R¹ is attached, the carbon atom to which R⁶ is attached, and the carbon atom contiguous to said nitrogen atom to which R¹ is attached and said carbon atom to which R⁶ is attached, to form a 5-membered or 6-membered, aromatic or dihydro-aromatic ring having carbon atoms and 1 or 2 nitrogen atoms;

R¹¹ is hydrogen, hydroxy, —CO₂H, or N(R¹²)R¹³,

R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;

m is an integer of 0 or 1; and

n is an integer selected from 0, 1, 2, 3, 4. This is hereinafter referred to as Process Embodiment 1A1.

Another embodiment of the present invention is a process of Process Embodiment 1A, or any one of the other above embodiments of a process of Process Embodiment 1A, wherein the compound of Formula I is a compound of formula

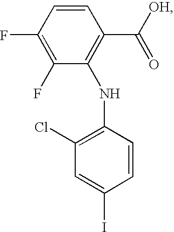

or a pharmaceutically acceptable salt thereof.

Another embodiment of the present invention is a process of Process Embodiment 1A1, wherein the compound of Formula I is a compound of formula

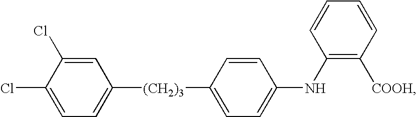

or a pharmaceutically acceptable salt thereof.

Another embodiment of the present invention is a process of Process Embodiment 1A1, wherein the compound of Formula I is a compound of formula

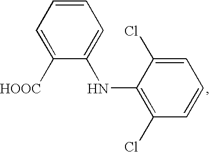

or a pharmaceutically acceptable salt thereof.

Another embodiment of the present invention is a process of Process Embodiment 1A1, wherein the compound of Formula I is a compound of formula

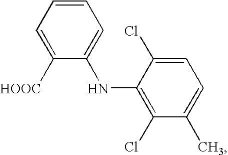

or a pharmaceutically acceptable salt thereof.

Another embodiment of the present invention is a process of Process Embodiment 1A1, wherein the compound of Formula I is a compound of formula

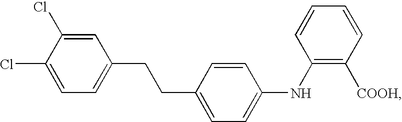

or a pharmaceutically acceptable salt thereof.

Another embodiment of the present invention is a process of Process Embodiment 1A1, wherein the compound of Formula I is a compound of formula

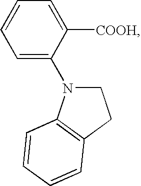

or a pharmaceutically acceptable salt thereof.

Another embodiment of the present invention is a process of Process Embodiment 1A1, wherein the compound of Formula I is a compound of formula

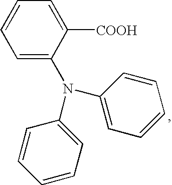

or a pharmaceutically acceptable salt thereof.

Another embodiment of the present invention is a process of Process Embodiment 1A1, wherein the compound of Formula I is a compound of formula

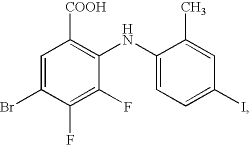

or a pharmaceutically acceptable salt thereof.

Another embodiment of the present invention is a process of Process Embodiment 1A1, wherein the compound of Formula I is a compound of formula

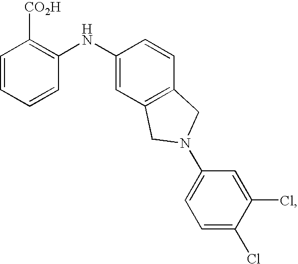

or a pharmaceutically acceptable salt thereof.

Another embodiment of the present invention is a process of Process Embodiment 1A1, wherein the compound of Formula I is a compound of formula

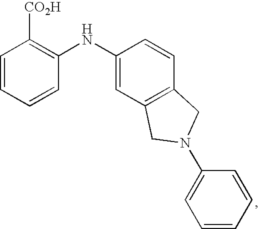

or a pharmaceutically acceptable salt thereof.

Another embodiment of the present invention is a process, hereinafter referred to as Process Embodiment 2, of synthesizing a compound of Formula Ie

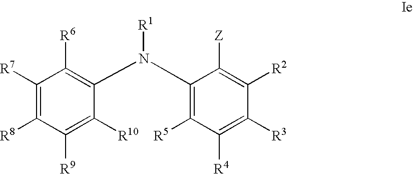

or a pharmaceutically acceptable salt thereof,

wherein:

R¹ is hydrogen, alkyl, alkoxy, or aryl;

R², R³, R⁴, R⁵, R⁶, R⁷, R⁸, R⁹, and R¹⁰ are each independently selected from:

hydrogen,

halo,

alkyl,

aryl,

a heterocyclic group,

haloalkyl,

alkoxy,

nitro,

CN,

—(O)ₘ—(CH₂)ₙ—R¹¹, or

—[N(H)]ₘ—(CH₂)ₙ—R¹¹, wherein m, n, and R¹¹ are as defined below, or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms,

or R¹ and R⁶ may be taken together with the nitrogen atom to which R¹ is attached, the carbon atom to which R⁶ is attached, and the carbon atom contiguous to said nitrogen atom to which R¹ is attached and said carbon atom to which R⁶ is attached, to form a 5-membered or 6-membered, aromatic or dihydro-aromatic ring having carbon atoms and 1 or 2 nitrogen atoms;

R¹¹ is hydrogen, hydroxy, —CO₂H, or N(R¹²)R¹³,

R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;

m is an integer of 0 or 1;

n is an integer selected from 0, 1, 2, 3, 4; and

Z is COOR¹⁵, —C(O)N(R¹⁶)R¹⁷, or —C(O)N(R¹⁸)OR¹⁹, wherein

R¹⁵ is alkyl, alkenyl, alkynyl, aryl, or a heterocyclic group, and

R¹⁶, R¹⁷, R¹⁸, and R¹⁹ are each independently selected from hydrogen, alkyl, alkenyl, phenyl, and benzyl, or

R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;

comprising coupling a compound of Formula If

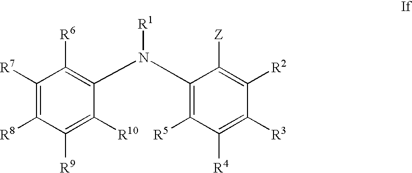

wherein Z is COOH or COOM, wherein M is a Group I metal cation or a hemi Group II metal cation, and R¹, R², R³, R⁴, R⁵, R⁶, R⁷, R⁸, R⁹, and R¹⁰ are as defined above, or when Z is COOM, R¹ is optionally a Group I metal cation or a hemi Group II metal cation,

with a compound of Formula II

HOR¹⁵ II

wherein R¹⁵ is as defined above, or a pharmaceutically acceptable salt thereof, or with a compound of Formula III

HN(R¹⁶)R¹⁷ III

or a pharmaceutically acceptable salt thereof, wherein R¹⁶ and R¹⁷ are as defined above, or with a compound of Formula IV

HN(R¹⁸)OR¹⁹ IV

or a pharmaceutically acceptable salt thereof, wherein R¹⁸ and R¹⁹ are as defined above.

Another embodiment of the present invention is a process of Process Embodiment 2, wherein R¹⁸ is hydrogen and R¹⁹ is selected from methyl, ethyl, propyl, isopropyl, 1-butyl, 2-butyl, 2-methyl-prop-1-yl, 1,1-dimethylethyl, 1-buten-1-yl, 1-buten-2-yl, 1-buten-3-yl, 1-buten-4-yl, 2-buten-1-yl, 2-buten-2-yl 1-methylcyclopropyl, 2-methylcyclopropyl, 1-methylcyclobutyl, 2-methylcyclobutyl, 3-methylcyclobutyl, 1-methylcyclopentyl, 2-methylcyclopentyl, 3-methylcyclopentyl, 1-methylcyclohexy, 2-methylcyclohexyl, 3-methylcyclohexyl, 4-methylcyclohexyl, cyclopropylmethyl, cyclopropyl-difluoromethyl, cyclopropyl-difluoromethyl, cyclobutylmethyl, cyclopentylmethyl, cyclohexylmethyl, phenyl, and benzyl.

Another embodiment of the present invention is a process of Process Embodiment 2, wherein R¹⁸ is hydrogen and R¹⁹ is cyclopropylmethyl.

Another embodiment of the present invention is a process of Process Embodiment 2, or any one of the other above embodiments of a process of Process Embodiment 2, wherein the compound of Formula I is a compound of formula

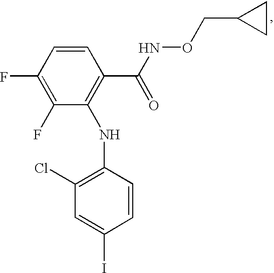

or a pharmaceutically acceptable salt thereof.

Another embodiment of the present invention is a process of Process Embodiment 2, wherein R¹⁶ is hydrogen and R¹⁷ is cyclopropylmethyl, 2-cyclopropylethyl, cyclobutylmethyl, 2-cyclobutylethyl, cyclopentylmethyl, 2-cyclopentylethyl, cyclohexylmethyl, 2-cyclohexylethyl, cyclopropyl-difluoromethyl, or 2-cyclopropyl-1,1-difluoroethyl.

Another embodiment of the present invention is a process of Process Embodiment 2, which hereinafter is referred to as a PROCESS OF EMBODIMENT 2a, of synthesizing a compound of Formula Ig

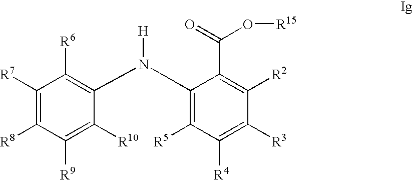

or a pharmaceutically acceptable salt thereof,

or a compound of Formula Ih

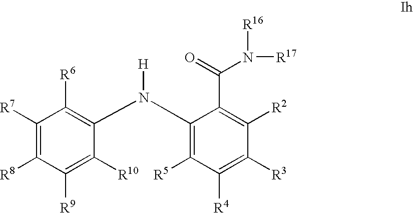

or a pharmaceutically acceptable salt thereof,

or a compound of Formula Ii

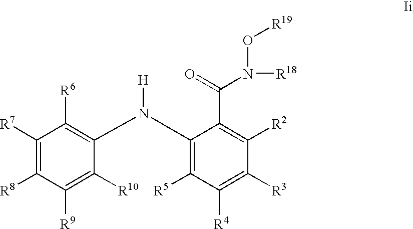

or a pharmaceutically acceptable salt thereof,

wherein:

R², R³, R⁴, R⁵, R⁶, R⁷, R⁸, R⁹, and R¹⁰ are each independently selected from:

hydrogen,

halo,

alkyl,

aryl,

a heterocyclic group,

haloalkyl,

alkoxy,

nitro,

CN,

—(O)ₘ—(CH₂)ₙ—R¹¹, or

—[N(H)]ₘ—(CH₂)ₙ—R¹¹, wherein m, n, and R¹¹ are as defined below, or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms;

R¹¹is hydrogen, hydroxy, —CO₂H, or N(R¹²)R¹³,

R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;

m is an integer of 0 or 1;

n is an integer selected from 0, 1, 2, 3, 4; and

R¹⁵ is alkyl, alkenyl, alkynyl, aryl, or a heterocyclic group,

R¹⁶, R¹⁷, R¹⁸, and R¹⁹ are each independently selected from hydrogen, alkyl, alkenyl, phenyl, and benzyl, or

R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl comprising reacting an acid selected from trifluoroacetic acid, trichloroacetic acid, a mineral acid, an alkylsulfonic acid, and an arylsulfonic acid

with a compound of Formula Ij

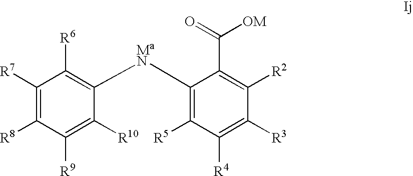

wherein R², R³, R⁴, R⁵, R⁶, R⁷, R⁸, R⁹, and R¹⁰ are as defined above,

M and Ma are each independently a Group I metal cation or a hemi Group II metal cation;

adding a carboxylic acid activating reagent to the mixture of Step (a), and reacting for a time, and at a temperature, sufficient to form a corresponding activated carboxylic acid intermediate; and

adding, optionally in the presence of up to 10 mol equivalents of a tertiary organic amine, a reactant which is selected from:

a compound of Formula II

HOR¹⁵ II

or a pharmaceutically acceptable salt thereof, wherein R¹⁵ is as defined above, or

a compound of Formula III

HN(R¹⁶)R¹⁷ III

or a pharmaceutically acceptable salt thereof, wherein R¹⁶ and R¹⁷ are as defined above, or

a compound of Formula IV

HN(R¹⁸)OR¹⁹ IV

or a pharmaceutically acceptable salt thereof, wherein R¹⁸ and R¹⁹ are as defined above, and reacting for a time, and at a temperature, sufficient to provide a compound of Formula Ig, Ih, or Ii, respectively.

Another embodiment of the present invention is a process of Process Embodiment 2a, wherein M^{a} is selected from lithium cation, sodium cation, and potassium cation.

Another embodiment of the present invention is a process of Process Embodiment 2a, wherein M^{a} is lithium cation.

Another embodiment of the present invention is a process of Process Embodiment 2a, wherein in Step (a), the acid employed is trifluoroacetic acid, trichloroacetic acid, a mineral acid selected from HCl, HBr, or H₂SO₄, an alkylsulfonic acid selected from CH₃SO₃H and CF₃SO₃H, or an arylsulfonic acid selected from phenyl-SO₃H and para-toluenesulfonic acid.

Another embodiment of the present invention is a process of Process Embodiment 2a, wherein in Step (a), the acid employed is CH₃SO₃H.

Another embodiment of the present invention is a process of Process Embodiment 2a, wherein the carboxylic acid activating reagent employed in Step (b) is selected from: (COCl)₂, S(O)Cl₂, S(O)₂Cl₂, P(O)Cl₃, (phenyl)₂P(═O)Cl, 1,1′-carbonyldiimidazole, triphenylphosphine/diethylazodicarboxylate, EDC, EDCI, and N,N′-dicyclohexylcarbodiimide.

Another embodiment of the present invention is a process of Process Embodiment 2a, wherein the carboxylic acid activating reagent employed in Step (b) is S(O)Cl₂.

Another embodiment of the present invention is a process of Process Embodiment 2a, wherein the carboxylic acid activating reagent employed in Step (b) is (phenyl)₂P(═O)Cl.

Another embodiment of the present invention is a process of Process Embodiment 2a, or any one of the other above embodiments of the process of Process Embodiment 2a, wherein the reactant added in step (c) is O-cyclopropylmethyl-hydroxylamine, or a pharmaceutically acceptable acid addition salt thereof.

Another embodiment of the present invention is a process of Process Embodiment 2a, or any one of the other above embodiments of the process of Process Embodiment 2a, wherein the compound of Formula I is a compound of formula

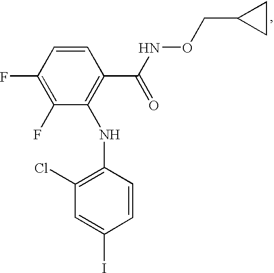

or a pharmaceutically acceptable salt thereof.

Another embodiment of the present invention is a process, hereinafter referred to as Process Embodiment 3, of synthesizing a compound of Formula Ik

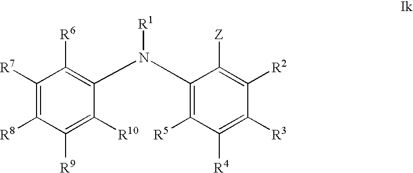

or a pharmaceutically acceptable salt thereof,

wherein:

R¹ is hydrogen, alkyl, alkoxy, or aryl;

R², R³, R⁴, R⁵, R⁶, R⁷, R⁸, R⁹, and R¹⁰ are each independently selected from:

hydrogen,

halo,

alkyl,

aryl,

a heterocyclic group,

haloalkyl,

alkoxy,

nitro,

CN,

—(O)ₘ—(CH₂)ₙ—R¹¹, or

—[N(H)]ₘ—(CH₂)ₙ—R¹¹, wherein m, n, and R¹¹ are as defined below, or

any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms, or

R¹ and R⁶ may be taken together with the nitrogen atom to which R¹ is attached, the carbon atom to which R⁶ is attached, and the carbon atom contiguous to said nitrogen atom to which R¹ is attached and said carbon atom to which R⁶ is attached, to form a 5-membered or 6-membered, aromatic or dihydro-aromatic ring having carbon atoms and 1 or 2 nitrogen atoms;

R¹¹ is hydrogen, hydroxy, —CO₂H, or N(R¹²)R¹³,

R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;

m is an integer of 0 or 1;

n is an integer selected from 0, 1, 2, 3, 4; and

Z is COOR¹⁵ wherein R¹⁵ is alkyl, alkenyl, alkynyl, aryl, or a heterocyclic group;

comprising coupling a compound of Formula If

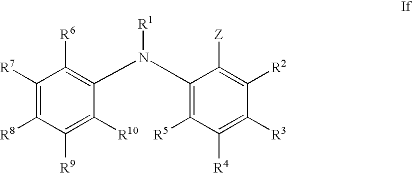

wherein Z is COOH or COOM, wherein M is a Group I metal cation or a hemi Group II metal cation, and R¹, R², R³, R⁴, R⁵, R⁶, R⁷, R⁸, R⁹, and R¹⁰ are as defined above, or when Z is COOM, R¹ is optionally a Group I metal cation or a hemi Group II metal cation,

with a compound of Formula II

HOR¹⁵ II

or a pharmaceutically acceptable salt thereof, wherein R¹⁵ is as defined above, optionally in the presence of an acid catalyst;

or with a compound of Formula IIa

L-R¹⁵ IIi

or a pharmaceutically acceptable salt thereof, wherein R¹⁵ is as defined above and L is a leaving group selected from bromo, chloro, iodo, alkylsulfonyloxy, and arylsulfonyloxy, acyloxy, optionally in the presence of a non-nucleophilic base.

Another embodiment of the present invention is a process, hereinafter referred to as Process Embodiment 4, for synthesizing a compound of Formula I

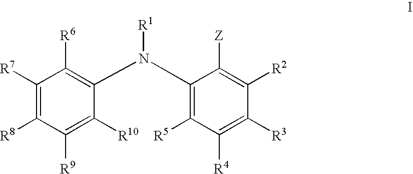

or a pharmaceutically acceptable salt thereof,

wherein:

R¹ is hydrogen, alkyl, alkoxy, or aryl;

R², R³, R⁴, R⁵, R⁶, R⁷, R⁸, R⁹, and R¹⁰ are each independently selected from:

hydrogen,

halo,

alkyl,

aryl,

a heterocyclic group,

haloalkyl,

alkoxy,

nitro,

CN,

—(O)ₘ—(CH₂)ₙ—R¹¹, or

—[N(H)]ₘ—(CH₂)ₙ—R¹¹, wherein m, n, and R ¹I are as defined below, or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms, or

R¹ and R⁶ may be taken together with the nitrogen atom to which R¹ is attached, the carbon atom to which R⁶ is attached, and the carbon atom contiguous to said nitrogen atom to which R¹ is attached and said carbon atom to which R⁶ is attached, to form a 5-membered or 6-membered, aromatic or dihydro-aromatic ring having carbon atoms and 1 or 2 nitrogen atoms;

R¹¹ is hydrogen, hydroxy, —CO₂H, or N(R¹²)R¹³,

R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;

m is an integer of 0 or 1;

n is an integer selected from 0, 1, 2, 3, 4; and

Z is COOH, COOM, COOR¹⁵, —C(O)R¹⁵, —C(O)N(R¹⁶)R¹⁷, —C(O)N(R¹⁸)OR¹⁹, NO₂, or CN, wherein

M is a Group I metal cation or a hemi Group II metal cation,

R¹⁵ is alkyl, alkenyl, alkynyl, aryl, or a heterocyclic group, and

R¹⁶, R¹⁷, R¹⁸, and R¹⁹ are each independently selected from hydrogen, alkyl, alkenyl, phenyl, and benzyl, or

R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;

comprising:

(a) a step for reacting a compound of Formula (A)

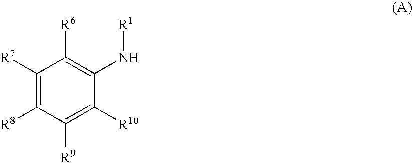

wherein R¹, R⁶, R⁷, R⁸, R⁹, and R¹⁰ are as defined above, with a compound of Formula (B)

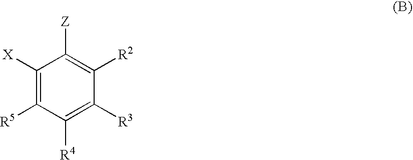

wherein Z, R², R³, R⁴, and R⁵ are as defined above, and X is halo or O-LG,

wherein LG is SO₂R²⁰ or P(═O)(OR²⁰)₂, wherein R²⁰ is alkyl or aryl, in the presence of from about 1 mol equivalent to about 10 mol equivalents of a base, wherein the base is selected from:

a Group I metal cation hydride or a Group 2 metal cation hydride, including lithium hydride, sodium hydride, potassium hydride, and calcium hydride,

a Group I metal cation dialkylamide or a Group 2 metal cation dialkylamide, including lithium diisopropylamide,

a Group I metal cation amide or a Group 2 metal cation amide, including lithium amide, sodium amide, potassium amide, and

a Group I metal cation alkoxide or a Group 2 metal cation alkoxide, including sodium ethoxide, potassium tert-butoxide, and magnesium ethoxide, and for a time, and at a temperature, sufficient to produce a compound of Formula I; and

(b) purifying the compound of Formula I produced in step (a).

Another embodiment of the present invention is a process, hereinafter referred to as Process Embodiment 5, for synthesizing a compound of Formula I

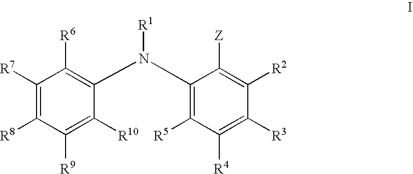

or a pharmaceutically acceptable salt thereof,

wherein:

R¹ is hydrogen, alkyl, alkoxy, or aryl;

R², R³, R⁴, R⁵, R⁶, R⁷, R⁸, R⁹, and R¹⁰ are each independently selected from:

hydrogen,

halo,

alkyl,

aryl,

a heterocyclic group,

haloalkyl,

alkoxy,

nitro,

CN,

—(O)ₘ—(CH₂)ₙ—R¹¹, or

—[N(H)]ₘ—(CH₂)ₙ—R¹¹, wherein m, n, and R¹¹ are as defined below, or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms, or

R¹ and R⁶ may be taken together with the nitrogen atom to which R¹ is attached, the carbon atom to which R⁶ is attached, and the carbon atom contiguous to said nitrogen atom to which R¹ is attached and said carbon atom to which R⁶ is attached, to form a 5-membered or 6-membered, aromatic or dihydro-aromatic ring having carbon atoms and 1 or 2 nitrogen atoms;

R¹¹ is hydrogen, hydroxy, —CO₂H, or N(R¹²)R¹³,

R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;

m is an integer of 0 or 1;

n is an integer selected from 0, 1, 2, 3, 4; and

Z is COOR¹⁵, —C(O)R¹⁵, —C(O)N(R¹⁶)R¹⁷, —C(O)N(R¹⁸)OR¹⁹, NO₂, or CN, wherein

R¹⁵ is alkyl, alkenyl, alkynyl, aryl, or a heterocyclic group, and

R¹⁶, R¹⁷, R¹⁸, and R¹⁹ are each independently selected from hydrogen, alkyl, alkenyl, phenyl, and benzyl, or

R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;

comprising:

(a) a step for reacting a compound of Formula (A)

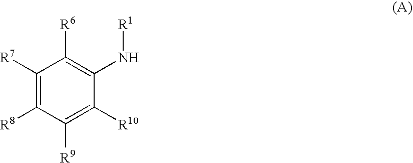

wherein R¹, R⁶, R⁷, R⁸, R⁹, and R¹⁰ are as defined above, with a compound of Formula (B)

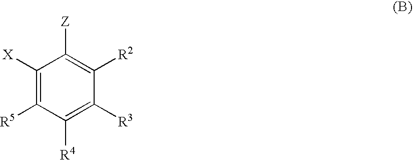

wherein Z, R², R³, R⁴, and R⁵ are as defined above, and X is halo or O-LG,

wherein LG is SO₂R²⁰ or P(═O)(OR²⁰)₂, wherein R²⁰ is alkyl or aryl, in the presence of from about 1 mol equivalent to about 10 mol equivalents of a base, wherein the base is a Group I metal cation bis(trialkylsilyl)amide or a Group 2 metal cation bis(trialkylsilyl)amide, including lithium bis(trimethylsilyl)amide, sodium bis(trimethylsilyl)amide, or potassium bis(trimethylsilyl)amide, and for a time, and at a temperature, sufficient to produce a compound of Formula I; and

(b) purifying the compound of Formula I produced in step (a).

Another embodiment of the present invention is a process, hereinafter referred to as Process Embodiment 6, for synthesizing a compound of Formula I

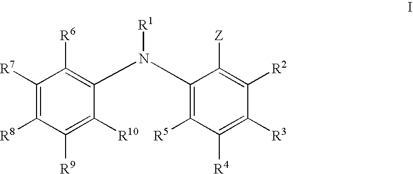

or a pharmaceutically acceptable salt thereof,

wherein:

R¹ is alkyl, alkoxy, or aryl;

R², R³, R⁴, R⁵, R⁶, R⁷, R⁸, R⁹, and R¹⁰ are each independently selected from:

hydrogen,

halo,

alkyl,

aryl,

a heterocyclic group,

haloalkyl,

alkoxy,

nitro,

CN,

—(O)ₘ—(CH₂)ₙ—R¹¹, or

—[N(H)]ₘ—(CH₂)ₙ—R¹¹, wherein m, n, and R¹¹ are as defined below,

or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms,

or R¹ and R⁶ may be taken together with the nitrogen atom to which R¹ is attached, the carbon atom to which R⁶ is attached, and the carbon atom contiguous to said nitrogen atom to which R¹ is attached and said carbon atom to which R⁶ is attached, to form a 5-membered or 6-membered, aromatic or dihydro-aromatic ring having carbon atoms and 1 or 2 nitrogen atoms;

R¹¹ is hydrogen, hydroxy, —CO₂H, or N(R¹²)R¹³,

R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;

m is an integer of 0 or 1;

n is an integer selected from 0, 1, 2, 3, 4; and Z is COOH or COOM;

comprising reacting a compound of Formula (A)

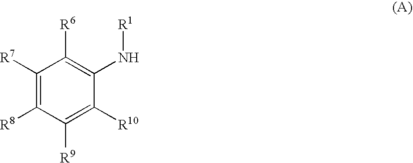

wherein R¹, R⁶, R⁷, R⁸, R⁹, and R¹⁰ are as defined above, with a compound of Formula (B)

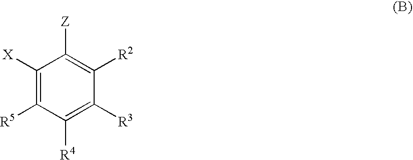

wherein Z, R², R³, R⁴, and R⁵ are as defined above, and X is halo or O-LG, wherein LG is SO₂R²⁰ or P(═O)(OR²⁰)₂, wherein R²⁰ is alkyl or aryl, optionally in a solvent, and in the presence of from about 1 mol equivalent to about 10 mol equivalents of a base, wherein the base is a Group I metal cation bis(trialkylsilyl)amide or a Group 2 metal cation bis(trialkylsilyl)amide, including lithium bis(trimethylsilyl)amide, sodium bis(trimethylsilyl)amide, or potassium bis(trimethylsilyl)amide, for a time, and at a temperature, sufficient to yield a compound of Formula I.

Another embodiment of the present invention is a process, hereinafter referred to as Process Embodiment 7, wherein the process is a process of any one of Process Embodiments 1, 1A, 2, 3, 4, 5, or 6 that is carried out on a commercial scale.

### DETAILED DESCRIPTION OF THE INVENTION

The present invention is methods of synthesizing a compound of Formula I

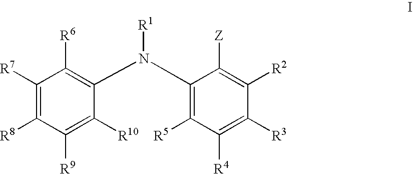

or a pharmaceutically acceptable salt thereof, wherein R¹, R², R³, R⁴, R⁵, R⁶, R⁷, R⁸, R⁹, R¹⁰, and Z are as defined above.

As used herein, the term “alkyl” means (i) a straight chain or branched chain hydrocarbon group having from 1 to 20 carbon atoms, (ii) a cyclic hydrocarbon group having from 3 to 20 carbon atoms, which is also known as a “cycloalkyl” group, (iii) a cyclic hydrocarbon group bonded through a straight chain or branched chain hydrocarbon group, which is also known as a “cycloalkyl-alkylene” group, wherein the total number of carbon atoms is from 4 to 20 and wherein alkylene is as defined below, or (iv) an alkyl group bonded through a cyclic alkylene, which is also known as an “alkyl-cycloalkylene” group, wherein the total number of carbon atoms is from 4 to 20 and wherein cycloalkylene is as defined below. Alkyl groups may be unsubstituted or substituted with from 1 to 4 substituents as described below. Preferred straight chain or branched chain alkyl groups have from 1 to 8 carbon atoms. Preferred cycloalkyl groups have from 3 to 8 carbon atoms. Other preferred alkyl groups have from 4 to 8 carbon atoms. C₁-C₆ alkyl means a straight chain or branched chain hydrocarbon group having from 1 to 6 carbon atoms. C₃-C₆ cycloalkyl means a cyclic hydrocarbon group having from 3 to 6 carbon atoms. Typical examples of straight chain or branched chain unsubstituted alkyl groups include methyl, ethyl, 1-propyl, 2-propyl, 1-butyl, 2-butyl, 2,2-dimethylethyl, 1-pentyl, 2-pentyl, 2,2-dimethylpropyl, 1-hexyl, 1-heptyl, 4-heptyl, 2-octyl, 2-methyl-hept-2-yl, 1-nonyl, 1-decyl, 1-undecyl, 1-dodecyl, 2-dodecyl, 2,4-dimethyl-2-decyl, 2-(1-methylethyl)-1-nonyl, 2-hexadecyl, and 1-tetradecyl. Illustrative examples of unsubstituted cycloalkyl groups are cyclopropyl, cyclobutyl, cyclopentyl, cyclohexyl, cycloheptyl, cyclooctyl, cyclononyl, cyclodecyl, cycloundecyl, cyclododecyl, cyclohexadecyl, and cyclotetradecyl. Illustrative examples of cycloalkyl-alkylene groups include cyclopropylmethyl, 3-cyclopentyl-hexyl, and 2-cyclopentyl-decyl. Illustrative examples of alkyl-cycloalkylene groups include 1-methyl-cyclopropyl, 3- hexyl-cyclopentyl, and 2-(dec-3-yl)-cyclopentyl. Substituted alkyl is described and illustrated below.

The term “alkenyl” means a straight chain or branched chain mono- or di-unsaturated hydrocarbon group having from 2 to 20 carbon atoms, or a cyclic mono-unsaturated hydrocarbon group having from 3 to 20 carbon atoms, which is also known as a “cycloalkenyl” group. Alkenyl groups may be unsubstituted or substituted with from 1 to 4 substituents as described below. Preferred straight chain or branched chain alkenyl groups have from 2 to 8 carbon atoms. Preferred cycloalkenyl groups have from 5 to 8 carbon atoms. Typical examples of straight chain or branched chain unsubstituted alkenyl groups include ethenyl, 1-propen-1-yl, 1-propen-2-yl, 2-propen-1-yl, 1-buten-3-yl, 1-butadienyl, 2-penten-2-yl, 1-hexen-6-yl, 1-hepten-3-yl, 3-hepten-1-yl, 2-octen-6-yl, 2-methyl-hept-2-en-4-yl, 1-nonen-8-yl, 1-decen-1-yl, 1-undecen-5-yl, CH₃—(CH₂)₁₀—C(H)═C(H)—C(H)═C(H)—, and 2,4-dimethyl-2-decen-1-yl. Illustrative examples of unsubstituted cycloalkenyl groups are 1-cyclopropenyl, 2-cyclobutenyl, 2-cyclopentenyl, 4-cyclohexenyl, 1-cycloheptenyl, 5-cyclooctenyl, 5-cyclononenyl, and 6-cyclotetradecenyl. Substituted alkenyl is described and illustrated below.

The term “alkynyl” means a straight chain or branched chain mono- or di-unsaturated hydrocarbon group having from 2 to 20 carbon atoms, or a cyclic mono-unsaturated hydrocarbon group having from 12 to 20 carbon atoms, which is also known as a “cycloalkynyl” group. Alkynyl groups may be unsubstituted or substituted with from 1 to 4 substituents as described below. Preferred straight chain or branched chain alkynyl groups have from 2 to 8 carbon atoms. Preferred cycloalkynyl groups have from 12 to 14 carbon atoms. Typical examples of straight chain or branched chain unsubstituted alkynyl groups include ethynyl, 1-propyn-1-yl, 1-propyn-3-yl, 2-propyn-1-yl, 1-butyn-3-yl, 1-butadiynyl, 2-pentyn-5-yl, 1-hexyn-6-yl, 1-heptyn-3-yl, 3-heptyn-1-yl, 2-octyn-6-yl, hept-2-yn-4-yl, and 4,4-dimethyl-2-decyn-1-yl. Illustrative examples of unsubstituted cycloalkynyl groups are 6-cyclotetradecynyl. Substituted alkynyl is described and illustrated below.

It should be appreciated that di-unsaturated, straight chain or branched chain, alkenyl and alkynyl groups may have as the second site of unsaturation a C≡C or C═C bond, respectively.

The term “alkoxy” means an alkyl group bonded through an oxygen atom, which is alkyl-O—, wherein alkyl is as defined above. Illustrative examples of unsubstituted alkoxy include methoxy, isopropoxy, 2-hexyloxy, cyclopropyloxy, cyclopentyloxy, and cyclohexyloxy. Illustrative examples of substituted alkoxy are provided below.

The term “acyl” means Rᵣ—C(O), wherein Rᵣ is hydrogen, alkyl, alkenyl, alkynyl, all as defined above, or aryl (including heteroaryl) as defined below. Illustrative examples of acyl include acetyl, benzoyl, 2-thienylcarbonyl, and cyclopentylcarbonyl. Illustrative examples of substituted acyl include hydroxyacetyl, 3,5-dichloro-4nitrobenzoyl, (2-methylphenyl)propylcarbonyl, and 3-hydroxycyclopentylcarbonyl.

The term “acyloxy” means Rᵣ—C(O)—O, wherein Rᵣ is hydrogen, alkyl, alkenyl, alkynyl, all as defined above, or aryl (including heteroaryl) as defined below. Illustrative examples of acyloxy include acetyloxy, benzoyloxy, 2-thienylcarbonyloxy, and cyclopentylcarbonyloxy. Illustrative examples of substituted acyloxy include hydroxyacetyloxy, trifluoroacetyloxy, 3,5-dichloro-4nitrobenzoyloxy, (2-methylphenyl)propylcarbonyloxy, and 3-hydroxycyclopentylcarbonyloxy.

The term “haloalkyl” means a halo bonded through an alkylene, which is a halo-alkylene group, wherein halo and alkylene are as defined below.

The terms “halo” and “halogen” may be used interchangeably, and mean fluoro, chloro, bromo, or iodo.

The phrases “hydroxamic ester” and hydroxamic acid ester” are synonymous and mean a hydroxamic acid that is substituted on the oxygen atom that is bonded to the nitrogen atom with the substituents described above for the group R¹⁹.

The term “alkylene” means a divalent, straight chain or branched chain, hydrocarbon group having from 1 to 20 carbon atoms, or a divalent cyclic hydrocarbon group having from 3 to 20 carbon atoms, each of which may be unsubstituted or substituted with from 1 to 4 substituents. Illustrative examples of an unsubstituted alkylene group include —CH₂—, —CH₂CH₂—, —CH₂CH₂CH₂—, —CH₂CH₂CH₂CH₂—, —C(CH₃)₂—CH₂—C(H)CH₃—(CH₂)₁₂—, —CH(CH₃)CH₂—, 1,4-cyclobutylene, 1,3-cyclohexylene, and 1,2-cycloheptadecylene. Illustrative examples of a substituted alkylene are provided below.

The term “alkylsulfonic acid” means an alkyl group bonded to a SO₃H group, which is also known as an alkyl-SO₃H group, wherein alkyl is unsubstituted alkyl as defined above, or is a fluoro-substituted alkyl. Illustrative examples of unsubstituted alkylsulfonic acids include CH₃SO₃H, ethanesulfonic acid, tert-butylsulfonic acid, and cyclohexylsulfonic acid. Illustrative examples of fluoro-substituted alkylsulfonic acid include CH₂FSO₃H, CF₂HSO₃H, CF₃SO₃H, and CH₃CF₂SO₃H.

The term “arylsulfonic acid” means an aryl group bonded to a SO₃H group, which is also known as an aryl-SO₃H group, wherein aryl is unsubstituted aryl or is aryl substituted with halo or unsubstituted alkyl, wherein halo and unsubstituted alkyl are as defined above.

The term “alkylsulfonyloxy” means an alkyl group bonded to a SO₃ group, which is also known as an alkyl-SO₃ group, wherein alkyl is unsubstituted alkyl as defined above, or is a fluoro-substituted alkyl. Illustrative examples of unsubstituted alkylsulfonic acids include CH₃SO₃, ethanesulfonyloxy, tert-butylsulfonyloxy, and cyclohexylsulfonyloxy. Illustrative examples of fluoro-substituted alkylsulfonyloxy include CH₂FSO₃, CF₂HSO₃, CF₃SO₃, and CH₃CF₂SO₃.

The term “arylsulfonyloxy” means an aryl group bonded to a SO₃ group, which is also known as an aryl-SO₃ group, wherein aryl is unsubstituted aryl or is aryl substituted with halo or unsubstituted alkyl, wherein halo and unsubstituted alkyl are as defined above.

As described above, alkyl, alkenyl, alkynyl, alkylene, alkoxy, and acyl may be substituted with from 1 to 4 substituents. The substituents are independently selected from:

phenyl,

phenyl substituted with from 1 to 3 substituents selected from C₁-C₆ alkyl, halo, OH, O—C₁-C₆ alkyl, 1,2-methylenedioxy, CN, NO₂, N₃, NH₂, N(H)CH₃, N(CH₃)₂, C(O)CH₃, OC(O)—C₁-C₆ alkyl, C(O)—H, CO₂H, CO₂—(C₁-C₆ alkyl), C(O)—N(H)OH, C(O)NH₂, C(O)NHMe, C(O)N(Me)₂, NHC(O)CH₃, N(H)C(O)NH₂, SH, S—C₁-C₆ alkyl, C≡CH, C(═NOH)—H, C(═NOH)—CH₃, CH₂OH, CH₂NH₂, CH₂N(H)CH₃, CH₂N(CH₃)₂, C(H)F—OH, CF₂—OH, S(O)₂NH₂, S(O)₂N(H)CH₃, S(O)₂N(CH₃)₂, S(O)—CH₃, S(O)₂CH₃, S(O)₂CF₃, or NHS(O)₂CH₃,

benzyl,

benzyl substituted with from 1 to 3 substituents selected from oxo (on methylene carbon only), C₁-C₆ alkyl, halo, OH, O—C₁-C₆ alkyl, 1,2-methylenedioxy, CN, NO₂, N₃, NH₂, N(H)CH₃, N(CH₃)₂, C(O)CH₃, OC(O)—C₁-C₆ alkyl, C(O)—H, CO₂H, CO₂—(C₁-C₆ alkyl), C(O)—N(H)OH, C(O)NH₂, C(O)NHMe, C(O)N(Me)₂, NHC(O)CH₃, N(H)C(O)NH₂, SH, S—C₁-C₆ alkyl, C≡CH, C(═NOH)—H, C(═NOH)—CH₃, CH₂OH, CH₂NH₂, CH₂N(H)CH₃, CH₂N(CH₃)₂, C(H)F—OH, CF₂—OH, S(O)₂NH₂, S(O)₂N(H)CH₃, S(O)₂N(CH₃)₂, S(O)—CH₃, S(O)₂CH₃, S(O)₂CF₃, or NHS(O)₂CH₃,

heteroaryl, wherein heteroaryl is as defined below,

heterocyclic group, wherein heterocyclic group is as defined below, oxo,

O—R_{Z}, wherein R_{Z} is hydrogen, C₁-C₆ alkyl, C₃-C₆ cycloalkyl, phenyl, or benzyl, wherein phenyl and benzyl may be substituted with from 1 to 3 substituents as described below,

S—R_{Z}, wherein R_{Z} is hydrogen, C₁-C₆ alkyl, C₃-C₆ cycloalkyl, phenyl, or benzyl, wherein phenyl and benzyl may be substituted with from 1 to 4 substituents as described below,

C(O)—R_{Z}, wherein R_{Z} is hydrogen, C₁-C₆ alkyl, C₃-C₆ cycloalkyl, phenyl, or benzyl, wherein phenyl and benzyl may be substituted with from 1 to 4 substituents as described below,

CO₂R_{Z}, wherein R_{Z} is hydrogen, C₁-C₆ alkyl, C₃-C₆ cycloalkyl, phenyl, or benzyl, wherein phenyl and benzyl may be substituted with from 1 to 4 substituents as described below,

C(O)—N(H)OR_{Z}, wherein R_{Z} is hydrogen, C₁-C₆ alkyl, phenyl, or benzyl,

C(═NOR_{Z})—H, wherein R_{Z} is hydrogen, C₁-C₆ alkyl, phenyl, or benzyl,

C(═NOR_{Z})—CH₃, wherein R_{Z} is hydrogen, C₁-C₆ alkyl, phenyl, or benzyl,

C(H)F—OH,

CF₂—OH,

O—C(O)—R_{Z}, wherein R_{Z} is hydrogen, C₁-C₆ alkyl, C₃-C₆ cycloalkyl, phenyl, or benzyl, wherein phenyl and benzyl may be substituted with from 1 to 4 substituents as described below,

C(O)—N(R_{Z})R_{y}, wherein R_{Z} and R_{y} are independently hydrogen, C₁-C₆ alkyl, C₃-C₆ cycloalkyl, phenyl, or benzyl, wherein phenyl and benzyl may be substituted with from 1 to 4 substituents as described below, or R_{Z} and R_{y} are taken together with the nitrogen atom to which they are attached to form a 5-membered, saturated heterocyclic ring having 1 nitrogen atom and 4 carbon atoms or a 6-membered, saturated heterocyclic ring of formula (Z), wherein (Z) is as defined above,

N(R_{Z})R_{y}, wherein R_{Z} and R_{y} are independently hydrogen, C₁-C₆ alkyl, C₃-C₆ cycloalkyl, phenyl, or benzyl, wherein phenyl and benzyl may be substituted with from 1 to 4 substituents as described below, or R_{Z} and R_{y} are taken together with the nitrogen atom to which they are attached to form a 5-membered, saturated heterocyclic ring having 1 nitrogen atom and 4 carbon atoms or a 6-membered, saturated heterocyclic ring of formula (Z), wherein (Z) is as defined above,

N(R_{Z})—C(O)—R_{y}, wherein R_{Z} and R_{y} are independently hydrogen, C₁-C₆ alkyl, C₃-C₆ cycloalkyl, phenyl, or benzyl, wherein phenyl and benzyl may be substituted with from 1 to 4 substituents as described below,

N(H)—C(O)—N(R_{Z})R_{y}, wherein R_{Z} and R_{y} are independently hydrogen, C₁-C₆ alkyl, phenyl, or benzyl, wherein phenyl and benzyl may be substituted with from 1 to 4 substituents as described below, or R_{Z} and R_{y} are taken together with the nitrogen atom to which they are attached to form a 5-membered, saturated heterocyclic ring having 1 nitrogen atom and 4 carbon atoms or a 6-membered, saturated heterocyclic ring of formula (Z), wherein (Z) is as defined above,

N(H)—C(O)—OR_{Z}, wherein R_{Z} is independently hydrogen, C₁-C₆ alkyl, C₃-C₆ cycloalkyl, phenyl, or benzyl, wherein phenyl and benzyl may be substituted with from 1 to 4 substituents as described below,

O—C(O)—N(R_{Z})R_{y}, wherein R_{Z} and R_{y} are independently hydrogen, C₁-C₆ alkyl, C₃-C₆ cycloalkyl, phenyl, or benzyl, wherein phenyl and benzyl may be substituted with from 1 to 4 substituents as described below, or R_{Z} and R_{y} are taken together with the nitrogen atom to which they are attached to form a 5-membered, saturated heterocyclic ring having 1 nitrogen atom and 4 carbon atoms or a 6-membered, saturated heterocyclic ring of formula (Z), wherein (Z) is as defined above,

(NO₂,

N₃,

N(H)—C(NR_{X})—N(R_{Z})R_{y}, wherein R_{Z} and R_{y} are independently hydrogen, C₁-C₆ alkyl, phenyl, or benzyl, wherein phenyl and benzyl may be substituted with from 1 to 4 substituents as described below, or R_{Z} and R_{y} are taken together with the nitrogen atom to which they are attached to form a 5-membered, saturated heterocyclic ring having 1 nitrogen atom and 4 carbon atoms or a 6-membered, saturated heterocyclic ring of formula (Z), wherein (Z) is as defined above, and R_{X} is hydrogen, hydroxy, methoxy, or CN,

CN,

halo,

S(O)—(C₁-C₆ alkyl),

S(O)₂—(C₁-C₆ alkyl),

S(O)₂—N(R_{Z})R_{y}, wherein R_{Z} and R_{y} are independently hydrogen, C₁-C₆ alkyl, C₃-C₆ cycloalkyl, phenyl, or benzyl, wherein phenyl and benzyl may be substituted with from 1 to 4 substituents as described below, or R_{Z} and R_{y} are taken together with the nitrogen atom to which they are attached to form a 5-membered, saturated heterocyclic ring having 1 nitrogen atom and 4 carbon atoms or a 6-membered, saturated heterocyclic ring of formula (Z), wherein (Z) is as defined above, and

N(H)—S(O)₂—(C₁-C₆ alkyl);

with the proviso that alkenyl or alkynyl are not substituted with oxo at unsaturated carbon atoms.

Illustrative examples of substituted straight chain or branched chain alkyl groups include CH₂OH, CF₂OH, CH₂C(CH₃)₂CO₂CH₃, CF₃, C(O)CF₃, C(O)—CH₃, (CH₂)₄—S—CH₃, CH(CO₂H)CH₂CH₂C(O)NMe₂, (CH₂)₅NH—C(O)—NH₂, CH₂—CH₂—C(H)—(4-fluorophenyl), CH(OCH₃)CH₂CH₃, (CH₂)₉-(morpholin-4-yl), CH₂SO₂NH₂, and CH(CH₃)CH₂CH₂OC(O)CH₃.

Illustrative examples of substituted cycloalkyl groups include 1-hydroxy-cyclopropyl, cyclobutanon-3-yl, 3-(3-phenyl-ureido)-cyclopent-1-yl, 4-carboxy-cyclohexyl, and 9-trifluromethyl-cyclododecanyl. Illustrative examples of substituted cycloalkyl-alkylene groups include cyclopropyl-difluoro-methyl, 2-cyclopropyl-1,1-difluoroethyl, cyclopropyl(methyl)methyl, 3-cyclopentyl-2-oxo-hexyl, and 2-cyclopentyl-1,1,1-trifluorodecyl. Illustrative examples of substituted alkyl-cycloalkylene groups include 1-trifluoromethyl-cyclopropyl, 3-hexyl-1-hydroxy-cyclopentyl, and 2-(2-cyano-dec-3-yl)-cyclopentyl. Substituted alkyl is described and illustrated below.

Illustrative examples of substituted straight chain or branched chain alkenyl groups include C(H)═C(H)CH₂OH, CH═CF₂, CH₂C(H)═C(H)—(CH₂)₂CF₂OH, CH₂C(═CH₂)CO₂CH₃, C(H)═C(H)—CF₃, CH₂—CH₂—C(H)═C(H)—C(O)—CH₃, C(H)═C(CH₃)—S—CH₃, C(H)═C(H)—C(H)═C(CH₃)—CO₂Me, (CH₂)₁₂—C(H)═C(H)—C(H)═C(H)-phenyl, CH₃—C≡C—CH₂—CH₂—C(H)═C(H)—, and C(H)═C═C(H)OC(O)CH₃.

Illustrative examples of substituted cycloalkenyl groups include 1-hydroxy-cyclopropenyl, 3-oxo-cyclobuten-1-yl, and 9-trifluromethyl-cyclododecen-1-yl.

Illustrative examples of substituted straight chain or branched chain alkynyl groups include C≡CCH₂OH, C≡CF, CH₂C≡C—(CH₂)₂CF₂OH, C≡C—CF₃, CH₂—CH₂—C≡C—C(O)—CH₃, C≡C—CH₂—S—CH₃, C≡C (CH₂)₈—CO₂Me, (CH₂)₁₂—C≡C-phenyl, CH₃—C≡C—CH₂—CH₂—C(H)═C(H)—, and C≡C—CH₂OC(O)CH₃. Illustrative examples of substituted cycloalkynyl groups include 4-trifluromethyl-cyclododecyn-4-yl.

Illustrative examples of substituted alkoxy include trifluoromethoxy, 2-carboxy-isopropoxy, 3-oxo-2-hexyloxy, (±)-2-methyl-cyclopropyloxy, (±)-3-amino-cyclopentyloxy, and 1-cyano-cyclohexyloxy.

Illustrative examples of substituted alkylene include hydroxymethylene, 2-dimethylaminobutylene, 2-fluoro-2-hexyl-propylene, and 2,4-cyclobutanone-diyl.

The term “aryl” means phenyl, substituted phenyl, 1-naphthyl, substituted 1-naphthyl, 2-napthyl, substituted 2-napthyl, or heteroaryl, wherein heteroaryl is as defined below. Substituted phenyl, substituted 1-naphthyl, and substituted 2-naphthyl groups are substituted with from 1 to 4 substituents as described below. Illustrative examples of substituted phenyl, substituted 1-naphthyl, and substituted 2-naphthyl are provided below.

The term “heteroaryl” means a 5-membered, monocyclic heteroaryl, a 6-membered, monocyclic heteroaryl, or a 9- or 10-membered, fused-bicyclic heteroaryl, which are as defined below, each of which may be unsubstituted or substituted as described below.

The phrase “5-membered, monocyclic heteroaryl” means an unsubstituted or substituted, 5-membered, monocyclic, aromatic ring group having carbon atoms and from 1 to 4 heteroatoms selected from N, O, and S, with the proviso that not more than 1 heteroatom atom which is O or S is present. Illustrative examples of an unsubstituted 5-membered, monocyclic heteroaryl include thiophen-2-yl, furan-2-yl, pyrrol-3-yl, pyrrol-1-yl, imidazol-4-yl, isoxazol-3-yl, oxazol-2-yl, thiazol-4-yl, tetrazol-1-yl, 1,2,4-oxadiazol-3-yl, 1,2,4-triazol-1-yl, and pyrazol-3-yl. Substituted 5-membered, monocyclic heteroaryl is described below.

The phrase “6-membered, monocyclic heteroaryl” means an unsubstituted or substituted, 6-membered, monocyclic, aromatic ring group having carbon atoms and 1 or 2 nitrogen atoms. Illustrative examples of an unsubstituted 6-membered, monocyclic heteroaryl include pyridin-2-yl, pyridin-4-yl, pyrimidin-2-yl, pyridazin-4-yl, and pyrazin-2-yl. Substituted 6-membered, monocyclic heteroaryl is described below.

The phrase “9- or 10-membered, fused-bicyclic heteroaryl” means an unsubstituted or substituted, 9-membered or 10-membered, fused-bicyclic, aromatic ring group having carbon atoms and from 1 to 4 heteroatoms selected from N, O, and S, with the proviso that not more than 2 heteroatoms which are oxygen atoms or sulfur atoms are present, and further that when 2 heteroatoms which are O and/or S are present, the O and/or S atoms are not bonded to each other. Illustrative examples of an unsubstituted 9- or 10-membered, fused-bicyclic heteroaryl include indol-2-yl, indol-6-yl, iso-indol-2-yl, benzimidazol-2-yl, benzimidazol-1-yl, benztriazol-1-yl, benztriazol-5-yl, quinolin-2-yl, isoquinolin-7-yl, benzopyrimidin-2-yl, benzoxazol-2-yl, benzothiophen-5-yl, and benzofuran-3-yl. Substituted 9- or 10-membered, bicyclic heteroaryl is described below.

The phrase “substituted 5-membered, monocyclic heteroaryl” means a 5-membered, monocyclic, aromatic ring group having carbon atoms and from 1 to 4 heteroatoms selected from N, O, and S, which is substituted with 1 or 2 substituents as defined below, with the proviso that not more than 1 heteroatom atom which is O or S is present, and further that each substituent is not bonded to an oxygen atom or a sulfur atom. Illustrative examples of a substituted, 5-membered, monocyclic heteroaryl are provided below.

The phrase “substituted 6-membered, monocyclic heteroaryl” means a 6-membered, monocyclic, aromatic ring group having carbon atoms and I or 2 nitrogen atoms, which is substituted with 1 or 2 substituents as defined below, with the proviso that each substituent is not bonded to a nitrogen atom. Illustrative examples of a substituted, 6-membered, monocyclic heteroaryl are provided below.

The phrase “substituted 9- or 10-membered, fused-bicyclic heteroaryl” means a 9-membered or 10-membered, fused-bicyclic, aromatic ring group having carbon atoms and from 1 to 4 heteroatoms selected from N, O, and S, which is substituted with from 1 to 3 substituents as defined below, with the proviso that not more than 2 heteroatoms which are O and/or S are present, and further that when 2 heteroatoms which are O and/or S atoms are present, the O and/or S atoms are not bonded to each other, and further that each substituent is not bonded to an oxygen atom or a sulfur atom. Illustrative examples of a substituted 9- or 10-membered, fused-bicyclic heteroaryl are provided below.

The phrase “heterocyclic group” means, except where otherwise noted, a heteroaryl, wherein heteroaryl is as defined above, or saturated or partially unsaturated 3- to 14-membered, monocyclic, bicyclic, or tricyclic ring having carbon atoms and from 1 to 5 heteroatoms selected from N, O, and S, which ring may be unsubstituted or substituted as defined below. The ring nitrogen atom(s) may be unprotected or protected with suitable nitrogen protecting groups. Preferred is a 5-membered, monocyclic heterocycloalkyl, a 6-membered, monocyclic heterocycloalkyl, or a 9- or 10-membered, fused-bicyclic heterocycloalkyl, which may be unsubstituted or substituted and are as defined below. Examples of useful heterocyclic groups included unsubstituted or substituted acridinyl, aziridinyl, benzathiaolinyl, benzimidazolyl, benzofuranyl, imidazolyl, 1H-indolyl, 1H-indazolyl, isoindolyl, isoquinolinyl, isothiazolyl, N-methyl-piperazinyl, morpholinyl, oxazolyl, 1,2,4-oxadiazolyl, phenazinyl, phenothiazinyl, phenoxazinyl, phthalazinyl, piperidinyl, piperazinyl, pteridinyl, purinyl, pyrazinyl, pyrazolyl, pyridazinyl, pyridinyl, pyrimidinyl, pyrrolyl, pyrrolidinyl, quinazolinyl, quinalinyl, quinoxalinyl, thiazolyl, 1,3,4-thiadiazolyl, thiophenyl, 1,3,5-triazinyl, 1,2,3-triazolyl, and the like.

The phrase “5-membered, monocyclic heterocycloalkyl” means a 5-membered, monocyclic nonaromatic ring group having carbon atoms and from 1 to 3 heteroatoms selected from nitrogen, oxygen, and sulfur, and optionally 1 double bond, and optionally substituted with 1 or 2 substituents as defined below, with the proviso that not more than 2 heteroatoms which are O and/or S atoms are present, and further when 2 heteroatoms which are O and/or S atoms are present, the O and/or S atoms are not bonded to each other. Preferred 5-membered, monocyclic heterocycloalkyl groups have no double bonds. Illustrative examples of unsubstituted 5-membered, monocyclic heterocycloalkyl include 2,3-dihydrofuran-2-yl, tetrahydrofuran-3-yl, and tetrahydroimidazol-1-yl. Substituted 5-membered, monocyclic heterocycloalkyl are described and illustrated below.

The phrase “6-membered, monocyclic heterocycloalkyl” means a 6-membered, monocyclic, nonaromatic ring group having carbon atoms and from 1 to 3 heteroatoms selected from nitrogen, oxygen, and sulfur, and optionally 1 or 2 double bonds, and optionally substituted with 1 or 2 substituents as defined below, with the proviso that not more than 2 heteroatoms which are O and/or S atoms are present, and further when 2 heteroatoms which are O and/or S atoms are present, the O and/or S atoms are not bonded to each other. Preferred 6-membered, monocyclic heterocycloalkyl groups have no double bonds. Illustrative examples of unsubstituted 6-membered, monocyclic heterocycloalkyl include 1,2,5,6-tetrahydropyridin-2-yl, piperidin-4-yl, piperazin-1-yl, morpholin-1-yl, and thiomorpholin-2-yl.

The phrase “9- or 10-membered, fused-bicyclic heterocycloalkyl” means a 9- or 10-membered, fused-bicyclic ring group having carbon atoms and from 1 to 4 heteroatoms selected from nitrogen, oxygen, and sulfur, wherein the fused-bicyclic ring has a first ring and a second ring, wherein the first ring is a 5- or 6-membered aromatic ring having carbon atoms and from 0 to 2 heteroatoms selected from nitrogen, oxygen, and sulfur, and the second ring is a 5- or 6-membered nonaromatic ring having carbon atoms and 1 or 2 heteroatoms selected from nitrogen, oxygen, and sulfur, and wherein the first ring and second ring are fused by sharing 1 double bond, (ie, the second ring is a dihydroaromatic ring), and optionally substituted with from 1 to 3 substituents as defined below, with the proviso that not more than 3 heteroatoms which are O and/or S atoms are present, and further that when 2 or 3 heteroatoms which are O and/or S atoms are present, the O and/or S atoms are not bonded to each other. Preferred 9- or 10-membered, fused-bicyclic heterocycloalkyl groups have a 6-membered ring fused to a 5-membered ring. Illustrative examples of unsubstituted 9- or 10-membered, fused-bicyclic heterocycloalkyl include 2,3-dihydro-benzofuran-2-yl and 2,3-dihydro-indol-5-yl.

The substituents for substituted aryl, which as shown above means substituted phenyl, substituted 1-naphthyl, substituted 2-naphthyl, substituted heteroaryl, which as shown above means a substituted 5-membered, monocyclic heteroaryl, a substituted 6-membered, monocyclic heteroaryl, or a substituted 9- or 10-membered, fused-bicyclic heteroaryl, or substituted heterocycloalkyl, which as shown above means substituted 5-membered, monocyclic heterocycloalkyl, substituted 6-membered, monocyclic heterocycloalkyl, or substituted 9- or 10-membered, fused-bicyclic heterocycloalkyl, are independently selected from:

C₁-C₆ alkyl,

C₂-C₆ alkenyl,

C₂-C₆ alkynyl,

C₃-C₆ cycloalkyl,

phenyl,

phenyl substituted with from 1 to 3 substituents selected from C₁-C₆ alkyl, halo, OH, O—C₁-C₆ alkyl, 1,2-methylenedioxy, CN, NO₂, N₃, NH₂, N(H)CH₃, N(CH₃)₂, C(O)CH₃, OC(O)—C₁-C₆ alkyl, C(O)—H, CO₂H, CO₂—(C₁-C₆ alkyl), C(O)—N(H)OH, C(O)NH₂, C(O)NHMe, C(O)N(Me)₂, NHC(O)CH₃, N(H)C(O)NH₂, SH, S—C₁-C₆ alkyl, C≡CH, C(═NOH)—H, C(═NOH)—CH₃, CH₂OH, CH₂NH₂, CH₂N(H)CH₃, CH₂N(CH₃)₂, C(H)F—OH, CF₂—OH, S(O)₂NH₂, S(O)₂N(H)CH₃, S(O)₂N(CH₃)₂, S(O)—CH₃, S(O)₂CH₃, S(O)₂CF₃, or NHS(O)₂CH₃,

benzyl,

benzyl substituted with from 1 to 3 substituents selected from oxo (on methylene carbon only), C₁-C₆ alkyl, halo, OH, O—C₁-C₆ alkyl, 1,2-methylenedioxy, CN, NO₂, N₃, NH₂, N(H)CH₃, N(CH₃)₂, C(O)CH₃, OC(O)—C₁-C₆ alkyl, C(O)—H, CO₂H, CO₂—(C₁-C₆ alkyl), C(O)—N(H)OH, C(O)NH₂, C(O)NHMe, C(O)N(Me)₂, NHC(O)CH₃, N(H)C(O)NH₂, SH, S—C₁-C₆ alkyl, C≡CH, C(═NOH)—H, C(═NOH)—CH₃, CH₂OH, CH₂NH₂, CH₂N(H)CH₃, CH₂N(CH₃)₂, C(H)F—OH, CF₂—OH, S(O)₂NH₂, S(O)₂N(H)CH₃, S(O)₂N(CH₃)₂, S(O)—CH₃, S(O)₂CH₃, S(O)₂CF₃, or NHS(O)₂CH₃,

(CH₂)₂₋₄-(phenyl),

(CH₂)₂₋₄-(substituted phenyl), wherein substituted phenyl is as defined immediately above,

O—R_{Z}, wherein R_{Z} is hydrogen, C₁-C₆ alkyl, phenyl, or benzyl,

S—R_{Z}, wherein R_{Z} is hydrogen, C₁-C₆ alkyl, phenyl, or benzyl, 1,2-methylenedioxy,

C(O)—R_{Z}, wherein R_{Z} is hydrogen, C₁-C₆ alkyl, phenyl, or benzyl,

CO₂R_{Z}, wherein R_{Z} is hydrogen, C₁-C₆ alkyl, phenyl, or benzyl,

C(O)—N(H)OR_{Z}, wherein R_{Z} is hydrogen, C₁-C₆ alkyl, phenyl, or benzyl,

C(═NOR_{Z})—H, wherein R_{Z} is hydrogen, C₁-C₆ alkyl, phenyl, or benzyl,

C(═NOR_{Z})—CH₃, wherein R_{Z} is hydrogen, C₁-C₆ alkyl, phenyl, or benzyl,

CH₂OR_{Z}, wherein R_{Z} is hydrogen, C₁-C₆ alkyl, phenyl, or benzyl,

CH₂N(R_{Z})R_{y}, wherein R_{Z} and R_{y} are independently hydrogen, C₁-C₆ alkyl, phenyl, or benzyl, or R_{Z} and R_{y} are taken together with the nitrogen atom to which they are attached to form a 5-membered, saturated heterocyclic ring having 1 nitrogen atom and 4 carbon atoms or a 6-membered, saturated heterocyclic ring of formula (Z), wherein (Z) is as defined above,

C(H)F—OH,

CF₂—OH,

O—C(O)—R_{Z}, wherein R_{Z} is hydrogen, C₁-C₆ alkyl, phenyl, or benzyl,

C(O)—N(R_{Z})R_{y}, wherein R_{Z} and R_{y} are independently hydrogen, C₁-C₆ alkyl, phenyl, or benzyl, or R_{Z} and R_{y} are taken together with the nitrogen atom to which they are attached to form a 5-membered, saturated heterocyclic ring having 1 nitrogen atom and 4 carbon atoms or a 6-membered, saturated heterocyclic ring of formula (Z), wherein (Z) is as defined above,

N(R_{Z})R_{y}, wherein R_{Z} and R_{y} are independently hydrogen, C₁-C₆ alkyl, phenyl, or benzyl, or R_{Z} and R_{y} are taken together with the nitrogen atom to which they are attached to form a 5-membered, saturated heterocyclic ring having 1 nitrogen atom and 4 carbon atoms or a 6-membered, saturated heterocyclic ring of formula (Z), wherein (Z) is as defined above,

N(R_{Z})—C(O)—R_{y}, wherein R_{Z} and R_{y} are independently hydrogen, C₁-C₆ alkyl, phenyl, or benzyl,

N(H)—C(O)—N(R_{Z})R_{y}, wherein R_{Z} and R_{y} are independently hydrogen, C₁-C₆ alkyl, phenyl, or benzyl, or R_{Z} and R_{y} are taken together with the nitrogen atom to which they are attached to form a 5-membered, saturated heterocyclic ring having 1 nitrogen atom and 4 carbon atoms or a 6-membered, saturated heterocyclic ring of formula (Z), wherein (Z) is as defined above,

N(H)—C(O)—OR_{Z}, wherein R_{Z} is independently hydrogen, C₁-C₆ alkyl, phenyl, or benzyl,

N(H)—S(O)₂—(C₁-C₆ alkyl),

O—C(O)—N(R_{Z})R_{y}, wherein R_{Z} and R_{y} are independently hydrogen, C₁-C₆ alkyl, phenyl, or benzyl, or R_{Z} and R_{y} are taken together with the nitrogen atom to which they are attached to form a 5-membered, saturated heterocyclic ring having 1 nitrogen atom and 4 carbon atoms or a 6-membered, saturated heterocyclic ring of formula (Z), wherein (Z) is as defined above,

NO₂,

N₃,

N(H)—C(NR_{X})—N(R_{Z})R_{y}, wherein R_{Z} and R_{y} are independently hydrogen, C₁-C₆ alkyl, phenyl, or benzyl, or R_{Z} and R_{y} are taken together with the nitrogen atom to which they are attached to form a 5-membered, saturated heterocyclic ring having 1 nitrogen atom and 4 carbon atoms or a 6-membered, saturated heterocyclic ring of formula (Z), wherein (Z) is as defined above, and R_{X} is hydrogen, hydroxy, methoxy, or CN,

CN,

halo,

S(O)—(C₁-C₆ alkyl),

S(O)₂—(C₁-C₆ alkyl),

S(O)₂—N(R_{Z})—(R_{y}), wherein R_{Z} and R_{y} are independently hydrogen, C₁-C₆ alkyl, phenyl, or benzyl, or R_{Z} and R_{y} are taken together with the nitrogen atom to which they are attached to form a 5-membered, saturated heterocyclic ring having 1 nitrogen atom and 4 carbon atoms or a 6-membered, saturated heterocyclic ring of formula (Z), wherein (Z) is as defined above, and

S(O)₂CF₃.

Preferred substituents for substituted aryl, and preferred substituents at carbon atoms for substituted 5-membered, monocyclic heteroaryl, substituted 6-membered, monocyclic heteroaryl, substituted 9- or 10-membered, fused-bicyclic heteroaryl, substituted 5-membered, monocyclic heterocycloalkyl, substituted 6-membered, monocyclic heterocycloalkyl, and substituted 9- or 10-membered, fused-bicyclic heterocycloalkyl are C₁-C₆ alkyl, halo, OH, O—C₁-C₆ alkyl, 1,2-methylenedioxy, CN, NO₂, N₃, NH₂, N(H)CH₃, N(CH₃)₂, C(O)CH₃, OC(O)—C₁-C₆ alkyl, C(O)—H, CO₂H, CO₂—(C₁-C₆ alkyl), C(O)—N(H)OH, C(O)NH₂, C(O)NHMe, C(O)N(Me)₂, NHC(O)CH₃, N(H)C(O)NH₂, SH, S—C₁-C₆ alkyl, C≡CH, C(═NOH)—H, C(═NOH)—CH₃, CH₂OH, CH₂NH₂, CH₂N(H)CH₃, CH₂N(CH₃)₂, C(H)F—OH, CF₂—OH, S(O)₂NH₂, S(O)₂N(H)CH₃, S(O)₂N(CH₃)₂, S(O)—CH₃, S(O)₂CH₃, S(O)₂CF₃, or NHS(O)₂CH₃.

Further as shown above, substituted 5-membered, monocyclic heteroaryl, substituted 9- or 10-membered, fused-bicyclic heteroaryl, substituted 5-membered, monocyclic heterocycloalkyl, substituted 6-membered, monocyclic heterocycloalkyl, and substituted 9- or 10-membered, fused-bicyclic heterocycloalkyl may optionally be substituted at a nitrogen atom, instead of a carbon atom, with 1 of certain substituents of said 1 or 2 substituents. Substitution at a nitrogen atom is possible when a ring —N(H)— is present. The substituent replaces the hydrogen atom in the diradical —N(H)— and is selected from:

C₁-C₆ alkyl, which may be straight or branched,

C₂-C₆ alkenyl, which may be straight or branched,

C₂-C₆ alkynyl, which may be straight or branched,

C₃-C₆ cycloalkyl, and

CN.

Still further, substituted 5-membered, monocyclic heterocycloalkyl, substituted 6-membered, monocyclic heterocycloalkyl, and substituted 9- or 10-membered, fused-bicyclic heterocycloalkyl may be substituted at a saturated carbon atom with oxo (═O) to form C═O.

As described above, substituted aryl means substituted phenyl, substituted 1-naphthyl, or substituted 2-naphthyl. Illustrative examples of:

(i) substituted phenyl include 4-methoxyphenyl, 2,6-difluorophenyl, 3-hydroxy-4-methylphenyl, 2-hydroxymethyl-3,4-dichloro-phenyl, 1,3-benzoxazol-5-yl, and 2-methoxy-4-nitrophenyl;

(ii) substituted l-naphthyl include 5-trifluoromethanesulfonylaminonaphth-1-yl and 2-(N-hydroxy-carboxamido)-naphth-1-yl; and

(iii) substituted 2-naphthyl includes 5-trifluoromethanesulfonylaminonaphth-2-yl and 1-(N-hydroxy-carboxamido)-naphth-2-yl.

As described above, substituted heteroaryl means substituted, 5-membered, monocyclic heteroaryl, substituted 6-membered, monocyclic heteroaryl, or substituted, 9- or 10-membered, fused-bicyclic heteroaryl. Illustrative examples of:

(i) substituted 5-membered, monocyclic heteroaryl include 3-chloro-thiophen-2-yl, 5-hexyl-furan-2-yl, 1-methyl-pyrrol-3-yl, 2-carboxy-pyrrol-1-yl, 1,2-dimethyl-imidazol-4-yl, 5-(4-carboethoxy-7-fluoro-heptyl)-isoxazol-3-yl, 4-trifluoromethyl-oxazol-2-yl, 2-hydroxy-thiazol-4-yl, 5-acetylamino-tetrazol-1-yl, 5-(tert-butyl)-1,2,4-oxadiazol-3-yl, 3-cyano-1,2,4-triazol-1-yl, and 5-acetyl-pyrazol-3-yl;

(ii) substituted 6-membered, monocyclic heteroaryl include 4,6-difluoro-pyridin-2-yl, 2-methyl-pyridin-4-yl, 4-azido-pyrimidin-2-yl, 6-ureido-pyridazin-4-yl, and 5-methylthio-pyrazin-2-yl; and

(iii) 9- or 10-membered, bicyclic heteroaryl include 6,7-dimethoxy-indol-2-yl, 1-propyl-indol-6-yl, 7-nitro-isoindol-2-yl, 1-benzyl-benzimidazol-2-yl, 4-chloro-benzimidazol-1-yl, 7-(2-propyl)-benztriazol-1-yl, 1-(2-hydroxyethyl)-benztriazol-5-yl, 4-iodo-quinolin-2-yl, 1-nitro-isoquinolin-7-yl, 4-cyano-benzopyrimidin-2-yl, 4,5,6-trifluoro-benzoxazol-2-yl, 2-carboxy-benzothiophen-5-yl, and 4-methylsulfinyl-benzofuran-3-yl.

As described above, substituted heterocycloalkyl means substituted, 5-membered, monocyclic heterocycloalkyl, substituted 6-membered, monocyclic heterocycloalkyl, or substituted, 9- or 10-membered, fused-bicyclic heterocycloalkyl. Illustrative examples of:

(i) substituted 5-membered, monocyclic heterocycloalkyl include 5-chloro-2,3-dihydrofuran-2-yl, 2,2-dimethyl-tetrahydrofuran-3-yl, 1-(3,4-dichloro-phenyl)-2,5-dihydro-1H-pyrrole-3,4-diyl (e.g., a 1-substituted, 2,5-dihydro-pyrrole benzo-fused at the 3,4-positions) and 2-oxo-tetrahydroimidazol-1-yl;

(ii) substituted 6-membered, monocyclic heterocycloalkyl include 4-acetyl-1,2,5,6-tetrahydropyridin-2-yl, 1-methyl -piperidin-4-yl, 4-benzyl-piperazin-1-yl, 3-fluoro-morpholin-1-yl, and 2-methyl-thiomorpholin-2-yl; and

(iii) 9- or 10-membered, bicyclic heterocycloalkyl include 4-nitro-2,3-dihydro-benzofuran-2-yl, 1,3-benzoxazol-5-yl, and 2-oxo-2,3-dihydro-indol-5-yl.

The term “heteroatom” means N, O, or S, except where otherwise noted.

The term “oxo” means ═O. Oxo, together with the carbon atom to which it is attached forms a carbonyl group (ie, C═O).

The term “amino” means NH₂.

The phrase “Group I metal cation” means Li⁺, Na⁺, K⁺, Rb⁺, Cs⁺, or Fr⁺.

The phrase “Group II metal cation” means Be⁺², Mg⁺², Ca⁺², Sr^{30 2}, Ba⁺², or Ra⁺².

The phrase “Group I metal cation amide” means a base which comprises NH₂⁻ and a cation which is Li⁺, Na⁺, K⁺, Rb⁺, Cs⁺, or Fr⁺.

The phrase “Group II metal cation amide” means a base which comprises NH₂⁻ and a cation which is Be⁺², Mg⁺², Ca⁺², Sr⁺², Ba⁺², or Ra⁺².

The phrase “Group I metal cation dialkylamide” means a base which comprises two independent alkyl groups each bonded to a N⁻ group, which is an alkyl-N(⁻)-alkyl group, wherein alkyl is unsubstituted alkyl as defined above, and a cation which is Li⁺, Na⁺, K⁺, Rb⁺, Cs⁺, or Fr⁺. Illustrative examples of a Group I metal cation dialkylamide includes lithium diisopropylamide (“LDA”).

The phrase “Group II metal cation dialkylamide” means a base which comprises two independent alkyl groups each bonded to a N⁻ group, which is an alkyl-N(⁻)-alkyl group, wherein alkyl is unsubstituted alkyl as defined above, and a cation which is Be⁺², Mg⁺², Ca⁺², Sr⁺², Ba⁺², or Ra⁺². Illustrative examples of a Group II metal cation dialkylamide includes magnesium bis(diisopropylamide).

The phrase “Group I metal cation bis(trialkylsilyl)amide” means a base which comprises two independent trialkylsilyl groups each bonded to a N⁻ group, which is an (alkyl)₃Si—N(⁻)—Si(alkyl)₃ group, wherein each alkyl is independently unsubstituted alkyl as defined above, and a cation which is Li⁺, Na⁺, K⁺, Rb⁺, Cs⁺, or Fr⁺. Illustrative examples of a Group I metal cation bis(trialkylsilyl)amide includes lithium bis(trimethylsilyl)amide (“LiHDMS” or “lithium hexamethyldisilazane”).

The phrase “Group II metal cation bis(trialkylsilyl)amide” means a base which comprises two independent trialkylsilyl groups each bonded to a N⁻ group, which is an (alkyl)₃Si—N(⁻)—Si(alkyl)₃ group, wherein each alkyl is independently unsubstituted alkyl as defined above, and a cation which is Be⁺², Mg⁺², Ca⁺², Sr⁺², Ba⁺², or Ra⁺². Illustrative examples of a Group II metal cation bis(trialkylsilyl)amide includes magnesium di[bis(trimethylsilyl)amide].

The phrase “Group I metal cation alkoxide” means a base which comprises an alkyl bonded to a O⁻ group, which is an alkyl-O⁻ group, wherein alkyl is unsubstituted alkyl as defined above, and a cation which is Li⁺, Na⁺, K⁺, Rb⁺, Cs⁺, or Fr⁺. Illustrative examples of a Group I metal cation alkoxide includes lithium methoxide, sodium ethoxide, and potassium tert-butoxide.

The phrase “Group II metal cation alkoxide” means a base which comprises an alkyl bonded to a O⁻ group, which is an alkyl-O⁻ group, wherein alkyl is unsubstituted alkyl as defined above, and a cation which is Be⁺², Mg⁺², Ca⁺², Sr⁺², Ba⁺², or Ra⁺². Illustrative examples of a Group II metal cation alkoxide includes magnesium bismethoxide and calcium bisethoxide.

Of the above mentioned bases, preferred are bases which comprise a salt of a Group I metal cation. More preferred are bases which comprise a salt of Li⁺, Na⁺, K⁺. Still more preferred are bases which comprise a salt of Li⁺. However, any base whereof the conjugate acid has a pKa≧16 will work in the invention process.

The term “comprising” which is synonymous with the terms “including”, “containing”, or “characterized by”, is inclusive or open-ended, and does not exclude additional, unrecited elements or method steps from the scope of the invention that is described following the term.

The phrase “consisting of” is closed-ended, and excludes any element, step, or ingredient not specified in the description of the invention that follows the phrase.

The phrase “consisting essentially of” limits the scope of the invention that follows to the specified elements, steps, or ingredients, and those further elements, steps, or ingredients that do not materially affect the basic and novel characteristics of the invention.

The phrase “carboxylic acid activating reagent” means a reagent which activates a —C(═O)OH group, or the corresponding conjugate base (ie, —C(═O)O⁻), towards a coupling reaction that involves displacement of the OH or O⁻, respectively. Illustrative examples of carboxylic acid activating reagents include lipase enzymes, mineral acids, including HCl and sulfuric acid, boron trifluoride etherate, 2,4,6-trichloro-1,3,5-triazine, 3-nitro-2-pyridinesulfenyl chloride, trifluoroacetic anhydride, mesyl chloride, S(O)Cl₂, S(O)₂Cl₂, P(O)Cl₃, oxalyl chloride, (phenyl)₂P(═O)Cl (“DPPCl”), 1,1′-carbonyldiimidazole (“CDI”), triphenylphosphine/diethylazodicarboxylate, N,N′-dicyclohexylcarbodiimide (“DCC”), the water soluble carbodiimides, including 1-(3-dimethylaminopropyl)-3-ethylcarbodiimide hydrochloride (“EDC”) and 1-(3-dimethylaminopropyl)-3-ethylcarbodiimide methiodide, 2-ethoxy-1-ethoxycarbonyl-1,2-dihydroquinoline (“EEDQ”), benzotriazol-1-yloxy-tris(dimethylamino)-phosphonium hexafluorophosphate (“BOP”), and bromo-tris(pyrrolidino)-phosphonium hexafluorophosphate (“PyBrOP”). Additional carboxylic acid activating reagents may be found in Comprehensive Organic Transformations, by Richard C. Larock, VCH Publishers, Inc, New York, 1989.

Preferred carboxylic acid activating reagents are selected from: (COCl)₂, S(O)Cl₂, S(O)₂Cl₂, P(O)Cl₃, (phenyl)2P(═O)Cl, 1,1′-carbonyldiimidazole, triphenylphosphine/diethylazodicarboxylate, EDC, EDCI, and N,N′-dicyclohexylcarbodiimide.

The phrase “organopalladium catalyst” means a catalyst comprising palladium and an organic ligand. Illustrative examples of organopalladium catalysts include palladium acetate, palladium tetrakis(triphenylphosphine), and palladium dichloride [bis(diphenylphosphino)ferrocene]. Other organopalladium catalysts are known, and may be found in Comprehensive Organic Transformations, by Richard C. Larock, VCH Publishers, Inc, New York, 1989.

The phrase “reactive functional group” means a group that is expected to react with certain solvents, reagents, catalysts, reaction starting materials, reaction intermediates, or reaction products under the particular reaction conditions employed. Examples of reactive functional groups include, but are not limited to, NH₂, OH, SH, CO₂H, N═C═O, C(O)Cl, and the like.

The phrase “non-nucleophilic base” means a base that is slow to act as a nucleophile in a substitution reaction such as, for example, a nucleophilic aromatic substitution reaction. Examples of non-nucleophilic bases include tertiary organic amines, which are defined below, Group I metal cation hydrides, Group II metal cation hydrides, Group I metal cation dialkylamides, Group II metal cation dialkylamides, Group I metal cation bis(trialkylsilyl)amides, Group II metal cation bis(trialkylsilyl)amides, Group I metal cation tertiary-alkoxides, and Group II metal cation tertiary-alkoxides.

The phrase “acid catalyst” means a Brnsted acid or Lewis acid which may be present in catalytic, stochiometric, or greater than stochiometric amounts.

The phrase “aprotic solvent” means a solvent that does not yield a proton (ie, acts as a Brnsted acid) under the particular conditions employed. This means that the pKa (relative to water or, optionally, DMSO) of an aprotic solvent is greater than the pKa of the conjugate acid of the strongest base employed. Typical aprotic solvents with high pKa's (ie, >30) include diethyl ether, tetrahydrofuran, dioxane, dimethylsulfoxide, hexane, heptane, dimethylformamide, toluene, and benzene. Typical aprotic solvents with lower pKa's (ie, 19<pKa<30) include ethyl acetate, acetone, and acetonitrile. Solvents with pKa's less than 19 such as, for example, tert-butyl alcohol, usually are not aprotic, although nitromethane is an aprotic solvent. Solvents that contain a functional group selected from OH, NH, and SH are typically not aprotic.

The phrases “protic solvent” or “protic contaminant” mean a solvent or contaminant, respectively, that does yield a proton under the particular conditions employed.

The phrase “tertiary organic amine” means a trisubstituted nitrogen group wherein the three substituents are independently selected from C₁-C₁₂ alkyl, C₃-C₁₂ cycloalkyl, and benzyl, or wherein two of the three substituents are taken together with the nitrogen atom to which they are attached to form a 5-membered or 6-membered, monocyclic heterocycle containing one nitrogen atom and carbon atoms and the third substituent is selected from C₁-C₁₂ alkyl, C₃-C₁₂ cycloalkyl, and benzyl, or wherein the three substituents are taken together with the nitrogen atom to which they are attached to form a 7-membered to 1 2-membered bicyclic heterocycle containing 1 or 2 nitrogen atoms total and carbon atoms, and optionally having a carbon-nitrogen double bond (“C═N”) when 2 nitrogen atoms are present. Illustrative examples of tertiary organic amine include triethylamine, diisopropylethylamine, benzyldiethylamine, dicyclohexyl-methyl-amine, 1,8-diazabicyclo[5.4.0]undec-7-ene (“DBU”), 1,4-diazabicyclo[2.2.2]octane (“TED”), and 1,5-diazabicyclo[4.3.0]non-5-ene.

The term “purifying” means separating a desired compound from undesired components of a mixture which contains both by methods which include distillation, chromatography, including column chromatography, thin layer chromatography, normal phase chromatography, reverse phase chromatography, gas phase chromatography, and ion exchange chromatography, precipitation, extraction, rotary evaporation, chemical-based trapping by reaction with an incompatible functional group, including quenching with polymer-bound quenching reagents, filtration, centrifugation, physical separation, and fractional crystallization.

The phrase “carried out on a commercial scale” means a process which employs more than 1 kilogram of a compound of formula (A) or a compound of formula (B), wherein a compound of formula (A) and a compound of formula (B) are as defined above.

Some of the compounds prepared according to a process of the present invention are capable of further forming pharmaceutically acceptable salts, including, but not limited to, acid addition and/or base salts. The acid addition salts are formed from basic compounds, whereas the base addition salts are formed from acidic compounds. All of these forms are within the scope of the compounds prepared by a process of the present invention.

Pharmaceutically acceptable acid addition salts of the basic compounds prepared according to a process of the present invention include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are salts of amino acids such as arginate and the like and gluconate, galacturonate (see, for example, Berge S. M. et al., “Pharmaceutical Salts,” J. of Pharma. Sci., 1977;66:1).

An acid addition salt of a basic compound prepared according to a process of the present invention is prepared by contacting the free base form of the compound with a sufficient amount of a desired acid to produce a nontoxic salt in the conventional manner. The free base form of the compound may be regenerated by contacting the acid addition salt so formed with a base, and isolating the free base form of the compound in the conventional manner. The free base forms of compounds prepared according to a process of the present invention differ from their respective acid addition salt forms somewhat in certain physical properties such as solubility, crystal structure, hygroscopicity, and the like, but otherwise free base forms of the compounds and their respective acid addition salt forms are equivalent for purposes of the present invention.

A pharmaceutically acceptable base addition salt of an acidic compound prepared according to a process of the present invention may be prepared by contacting the free acid form of the compound with a nontoxic metal cation such as an alkali or alkaline earth metal cation, or an amine, especially an organic amine. Examples of suitable metal cations include sodium cation (Na⁺), potassium cation (K⁺), magnesium cation (Mg⁺²), calcium cation (Ca⁺²), and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, supra., 1977).

A base addition salt of an acidic compound prepared according to a process of the present invention may be prepared by contacting the free acid form of the compound with a sufficient amount of a desired base to produce the salt in the conventional manner. The free acid form of the compound may be regenerated by contacting the salt form so formed with an acid, and isolating the free acid of the compound in the conventional manner. The free acid forms of the compounds prepared according to a process of the present invention differ from their respective salt forms somewhat in certain physical properties such as solubility, crystal structure, hygroscopicity, and the like, but otherwise the salts are equivalent to their respective free acid for purposes of the present invention.

Certain of the compounds prepared according to a process of the present invention can exist in unsolvated forms as well as solvated forms, including hydrated forms. In general, the solvated forms, including hydrated forms, are equivalent to unsolvated forms and are intended to be encompassed within the scope of the present invention.

Certain of the compounds prepared according to a process of the present invention possess one or more chiral centers, and each center may exist in the R or S configuration. A process of the present invention prepares all diastereomeric, enantiomeric, and epimeric forms of the compounds as well as mixtures thereof.

Additionally, certain compounds prepared according to a process of the present invention may exist as geometric isomers such as the entgegen (E) and zusammen (Z) isomers of alkenyl groups. A process of the present invention prepares all cis, trans, syn, anti, and entgegen (E), and zusammen (Z) isomers as well as mixtures thereof.

Certain compounds prepared according to a process of the present invention can exist as two or more tautomeric forms. Tautomeric forms of the compounds may interchange, for example, via enolization/de-enolization and the like. A process of the present invention prepares all tautomeric forms of the compounds of Formula I.

Intermediates for the synthesis of the compounds of Formula I, and pharmaceutically acceptable salts thereof, may be prepared by one of ordinary skill in the art of organic chemistry by adapting various synthetic procedures that are well known in the art of organic chemistry. These synthetic procedures may be found in the literature in, for example, Reagents for Organic Synthesis, by Fieser and Fieser, John Wiley & Sons, Inc, New York, 2000; Comprehensive Organic Transformations, by Richard C. Larock, VCH Publishers, Inc, New York, 1989; the series Compendium of Organic Synthetic Methods (1989) by Wiley-Interscience; the text Advanced Organic Chemistry, 4^{th} edition, by Jerry March, Wiley-Interscience, New York (1992); or the Handbook of Heterocyclic Chemistry by Alan R. Katritzky, Pergamon Press Ltd, London, 1985, to name a few. Alternatively, a skilled artisan may find methods useful for preparing the intermediates in the chemical literature by searching widely available databases such as, for example, those available from the Chemical Abstracts Service, Columbus, Ohio, or MDL Information Systems GmbH (formerly Beilstein Information Systems GmbH), Frankfurt, Germany.

Preparations of the compounds of the present invention may use starting materials, reagents, solvents, and catalysts that may be purchased from commercial sources or they may be readily prepared by adapting procedures in the references or resources cited above. Commercial sources of starting materials, reagents, solvents, and catalysts useful in preparing invention compounds include, for example, The Aldrich Chemical Company, and other subsidiaries of Sigma-Aldrich Corporation, St. Louis, Mo., BACHEM, BACHEM A.G., Switzerland, or Lancaster Synthesis Ltd, United Kingdom.

Syntheses of some compounds of the present invention may utilize starting materials, intermediates, or reaction products that contain a reactive functional group. During chemical reactions, a reactive functional group may be protected using protecting groups that render the reactive group substantially inert to the reaction conditions employed. A protecting group is introduced onto a starting material prior to carrying out the reaction step for which a protecting group is needed. Once the protecting group is no longer needed, the protecting group can be removed. It is well within the ordinary skill in the art to introduce protecting groups during a synthesis of a compound of Formula I, and then later remove them. Procedures for introducing and removing protecting groups are known and referenced such as, for example, in Protective Groups in Organic Synthesis, 2^{nd} ed., Greene T. W. and Wuts P. G., John Wiley & Sons, New York: N.Y., 1991, which is hereby incorporated by reference. Thus, for example, protecting groups such as the following may be utilized to protect amino, hydroxyl, and other groups: carboxylic acyl groups such as, for example, formyl, acetyl, and trifluoroacetyl; alkoxycarbonyl groups such as, for example, ethoxycarbonyl, tert-butoxycarbonyl (BOC), β,β,β-trichloroethoxycarbonyl (TCEC), and β-iodoethoxycarbonyl; aralkyloxycarbonyl groups such as, for example, benzyloxycarbonyl (CBZ), para-methoxybenzyloxycarbonyl, and 9-fluorenylmethyloxycarbonyl (FMOC); trialkylsilyl groups such as, for example, trimethylsilyl (TMS) and tert-butyldimethylsilyl (TBDMS); and other groups such as, for example, triphenylmethyl (trityl), tetrahydropyranyl, vinyloxycarbonyl, ortho-nitrophenylsulfenyl, diphenylphosphinyl, para-toluenesulfonyl (Ts), mesyl, trifluoromethanesulfonyl, and benzyl. Examples of procedures for removal of protecting groups include hydrogenolysis of CBZ groups using, for example, hydrogen gas at 50 psi in the presence of a hydrogenation catalyst such as 10% palladium on carbon, acidolysis of BOC groups using, for example, hydrogen chloride in dichloromethane, trifluoroacetic acid (TFA) in dichloromethane, and the like, reaction of silyl groups with fluoride ions, and reductive cleavage of TCEC groups with zinc metal.

General invention processes are illustrated in the Schemes below.

The process of the present invention for the synthesis of a compound of Formula I, or a pharmaceutically acceptable salt thereof via base-promoted reaction of a compound of Formula (A) with a compound of Formula (B), wherein R¹ to R¹⁰, X, and Z are as defined above, is outlined below in Scheme 1.

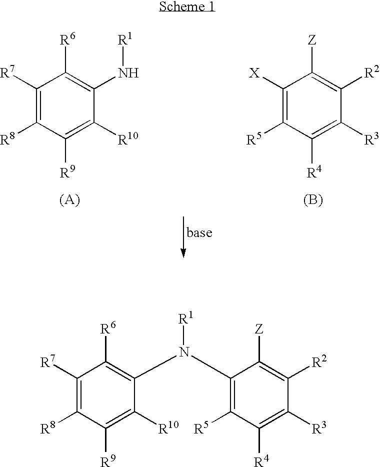

In Scheme 1, the number of mole-equivalents (“mol eq.”) of base used in the process of the present invention compared to the smaller of the number of moles of a compound of Formula (A) or a compound of Formula (B) used in the present invention is preferably greater than about 2, more preferably greater than about 2.5, still more preferably greater than about 2.75, and most preferably between 3 and 3.5. Reducing the number of equivalents below 3 decreases the yield except in cases wherein a compound of Formula (B), wherein Z is COOM, wherein M is Group I metal cation or a hemi Group II metal cation, is used as a starting material. In these cases, the preferable amount is between 2 and 2.5 equivalents of added base. The use of more than 3.5 equivalents of base improves the reaction yield in cases where the reagents contain a protic solvent or a protic contaminant (see Table I, entries 1-4 below).

In general, a reaction of the present invention is accomplished by mixing a compound of Formula (A) with a compound of Formula (B), preferably in an aprotic solvent, which solvent is preferably tetrahydrofuran or acetonitrile, along with a base. A reaction is generally carried out at a temperature of about −78° C. to about 150° C. (preferably about −70° C. to about 120° C.), and normally is complete within about 2 hours to about 4 days. A compound of Formula I produced by a process of the present invention can be isolated by removing the solvent, for example, by rotary evaporation under reduced pressure, and further purified, if desired, by standard methods such as chromatography, crystallization or distillation. Other standard purification methods are recited above.

The base employed in the process of the present invention can be added to the reaction in several ways. Four methods, namely Methods A-D, are described below.

Method A—The base can be added in a “two-pot procedure,” wherein a first flask, base is added to a solution or suspension of a compound of Formula (B) (˜1 mol eq.); in a second flask, base is added to a solution or suspension of a compound of Formula (A) (˜1 mol eq.). The contents can be combined, and the resulting mixture warmed, if necessary or desired, to react.

Method B—The base can be added in a “one-pot procedure,” where both compounds of Formulas (A) and (B) are dissolved or suspended, in solvent and cooled. The base is added, and the mixture is warmed, if necessary or desired, to react.

Method C—The base can be added in an alternate “two-pot procedure,” where in a first flask is a solution or suspension of a compound of Formula (B) (˜1 mol eq.); in a second flask, the base and a compound of Formula A (˜1 eq.) are mixed. The contents from the first flask are transferred into the second flask, or, optionally, the contents from the second flask are transferred into the first flask, and the resulting is mixture warmed, if necessary or desired, to react.

Method D—A solution or suspension of a compound of Formula (B) (˜1 eq.); and a compound of Formula (A) (˜1 eq.) is made, and the contents transferred into a flask containing the base or, optionally, the contents of the flask containing the base are transferred into the flask containing compounds of Formulas (A) and (B). The resulting mixture is warmed, if necessary or desired, to react.

When Z is —COOH, a compound of Formula I can be reacted with an alcohol (optionally in the presence of a coupling agent) to produce an ester.

When Z is —COOH, or —COOM, a compound of Formula I can be reacted with NH₃, a primary or secondary amine, hydroxylamine, or an O-substituted hydroxylamine to form an amide, hydroxamic acid, or a hydroxamic ester.

When a compound Formula I is intended to be further reacted with an alcohol, amine, or hydroxylamine derivative, to produce a pharmaceutical ester, amide (such as those described in WO 99/01421 and WO 99/01426) or hydroxamic acid or hydroxamic ester, then it is convenient to activate a compound of Formula I, wherein Z is COOH or COOM, wherein M is as defined above, in a manner which can avoid the need for isolation. In these instances, it is convenient to use a method as shown below in Scheme 2:

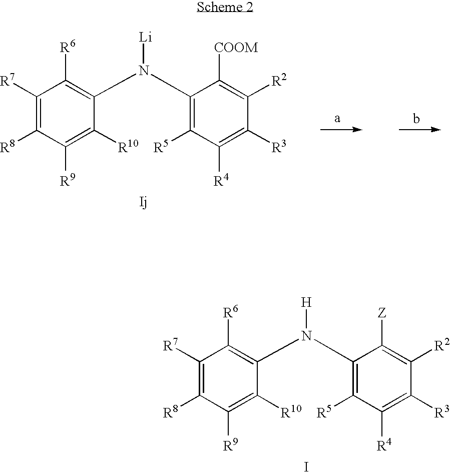

where in M and R² to R¹⁰ are as described above; and

Z is COOR′⁵, C(O)N(R¹⁶)R¹⁷, or C(O)N(R¹⁸)R¹⁹, wherein R¹⁵ to R¹⁹ are as defined above.

In Step (a) of Scheme 2, 1 mol eq. of an acid such as MeSO₃H is added at a temperature and for a time sufficient to monoprotonate a compound of Formula I, to give a carboxylate salt intermediate. Typically, the temperature is about −78° C. to about 0° C. (preferably about −20° C.), and the reaction is complete within about 30 minutes.

Thereafter, in Step (b), the carboxylate salt intermediate is converted to the corresponding acid chloride intermediate with a reagent such as thionyl chloride (SOCl₂). Subsequently, the acid chloride intermediate is reacted with an alcohol, amine, or a hydroxylamine derivative to obtain an ester, amide, or hydroxamic acid or hydroxamic ester, wherein Z is as defined immediately above.

In another embodiment of the present invention, higher yields of a compound of Formula I, obtained by reaction of a compound of Formula (A) with a compound of Formula (B), may be achieved by employing sequential addition of said base. In one such sequential addition procedure, the procedure comprises

(a) dissolving or suspending a compound of formula (A) and a compound of formula (B) in a solvent, preferably an aprotic solvent;

(b) adding a base to the mixture of Step (a), which mixture is preferably at a temperature of from about −70° C. to about 30° C., and allowing the compound of formula (A) and the compound of formula (B) to react for a time sufficient to increase the amount of a compound of Formula I;

(c) optionally, heating the reaction mixture of Step (b) for a time sufficient to increase the amount of a compound of Formula I or decrease the time required to produce an amount of a compound of Formula I;

(d) adding a base to the mixture of Step (b), which mixture is preferably at a temperature of from about −70° C. to about 30° C. and allowing to react for a time sufficient to increase the amount of a compound of Formula I; or cooling the mixture of step (c) to a temperature of from about −70° C. to about 30° C., adding a base to the cooled mixture, and allowing to react for a time sufficient to increase the amount of a compound of Formula I;

(e) optionally, heating the reaction mixture of step (d) for a time sufficient to increase the amount of a compound of Formula I; and

(f) optionally repeating steps (d) and (e).

The number of times that Step (d) or Steps (d) and (e) are repeated according to Step (f) is preferably less than 10, and is most preferably from 0 to 7 times.

The amount of base added in Step (b) is preferably about 2 mol equivalents, except in cases where a compound of Formula (B), wherein Z is COOM, wherein M is as defined above, is employed. Then the amount of base is preferably about 1 mol equivalent.

The amount of base added in Step (d) is preferably about 0.5 mol equivalents. As Step (d) is repeated, the number of mol equivalents of base should be decreased by about 50% compared to the number of mol eq. used previously.

The base used in Steps (b) and (d) above can be the same or different. Suitable bases include lithium diisopropylamide (LDA), lithium hydride, lithium amide, lithium diethylamide, lithium bis(trimethylsilyl)amide, sodium bis(trimethylsilyl)amide, or potassium bis(trimethylsilyl)amide.

LDA is preferably used in a process of the present invention in the commercial (ie, large-scale) setting, but more preferred bases are LiHMDS, LiH, or LiNH₂.

It should be appreciated that the process of the present invention can be conducted in any size container or reactor.

The following examples are provided merely to further illustrate the invention. The scope of the invention is not to be construed as merely consisting of the following examples.

### EXAMPLE 1

2-(2-Chloro-4-iodophenylamino)-3,4-difluorobenzoic acid

Sequential Addition Procedure Using Lithium Diisopropylamide (LDA) as the Base

Method B

To a 3 L flask fitted with a mechanical stirrer was added 58 g (0.33 mol) 2,3,4-trifluorobenzoic acid, 76 g (0.30 mol) 2-chloro-4-iodoaniline, and 500 mL THF. The mixture was cooled to about −20° C., and 400 mL of 1.5 M lithium diisopropylamide (“LDA”) solution in hexane/THF was added. The reaction was then allowed to warm to room temperature and stirred for at least 1 hour. The reaction was then cooled to about −20° C., and 100 mL of 1.5 M LDA solution in hexane/THF was added. The reaction was then allowed to warm to room temperature and stirred for at least 1 hour. The reaction was then cooled to about −20° C., and 50 mL of 1.5 M LDA solution in hexane/THF was added. The reaction was then allowed to warm to room temperature and stirred for at least 1 hour. The reaction was then cooled to about −20° C., and 25 mL of 1.5 M LDA solution in hexane/THF was added. The reaction was then allowed to warm to room temperature and stirred for at least 1 hour. The reaction was then cooled to about −20° C., and 12 mL of 1.5 M LDA solution in hexane/THF was added. The reaction was then allowed to warm to room temperature and stirred for at least 1 hour. The reaction was then cooled to about −20° C., and 6 mL of 1.5 M LDA solution in hexane/THF was added. The reaction was then allowed to warm to room temperature and stirred for at least 1 hour. The reaction was then cooled to about −40° C., and 600 mL of 4 M aqueous HCl was added. The reaction was then allowed to warm to room temperature and stirred for at least 10 minutes and the phases allowed to separate for at least 1 hour. The lower layer was then discarded and the upper layer concentrated by vacuum distillation to a slurry. The slurry was dissolved in hot acetone and the solution diluted with water and cooled to crystallize. The product was isolated by filtration and dried in a vacuum oven resulting in 96 g (78%) of 2-(2-chloro-4-iodophenylamino)-3,4-difluorobenzoic acid as an off-white solid.

Table 1 below presents the in-process high performance liquid chromatography (“HPLC”) results obtained during the preparation described in Example 1. The area percent amount of 2-(2-chloro-4-iodophenylamino)-3,4-difluorobenzoic acid is shown as determined by using an ultraviolet (“UV”) detector.

TABLE 1

| | | | | | | | |
|---|---|---|---|---|---|---|---|
| Molar | 2 | 0.5 | 0.25 | 0.13 | 0.06 | 0.03 | 0.015 |
| Equivalents | eq | eq | eq | eq | eq | eq | eq |
| of LDA | | | | | | | |
| Area Percent | 40% | 66% | 82% | 89% | 93% | 96% | 97% |
| Product | | | | | | | |

Additional Examples 2-10, indicated by their Example number in the column labelled “Ex. No.”, are shown below in Table 2. Results are shown as percent yield of a compound of Formula I in the column labelled “Yield (%)”. The reactants are a compound of formula (A) and a compound of formula (B), which are shown in the columns labelled “(A)” and “(B)”, respectively. The base and method employed are shown in the columns labelled “Base” and “Method”, respectively. Three (3) mole-equivalents of base were employed unless otherwise indicated.

TABLE 2

| Ex. | | | | | Yield |
|---|---|---|---|---|---|
| No. | (B) | (A) | Base | Method | (%) |
| 2 | 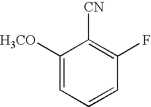 | 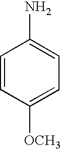 | LiHMDS | A | 33 |
| 3 | 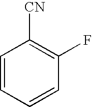 | 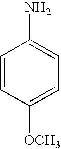 | LiHMDS | B | 6 |
| 4 | 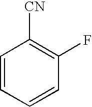 | 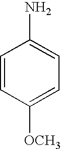 | LiHMDS | B | a |
| 5 | 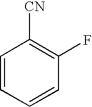 | 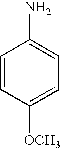 | LiHMDS | B | 6 |
| 6 | 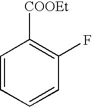 | 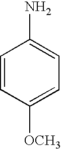 | LiHMDS | B | a |
| 7 | 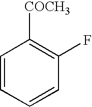 | 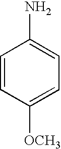 | LiHMDS | B | ND^{b} |
| 8 | 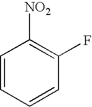 | 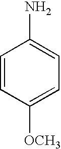 | LiHMDS | A | 78 |
| 9 | Same as | Same as | LiHMDS | A^{c} | 87 |
| | Ex. 8 | Ex. 8 | | | |
| 10 | 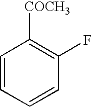 | 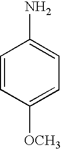 | LiHMDS | B | ND |

(a) Amide formation was observed
^{b}ND means amount of product was not determined
^{c}1.3 equivalents of a compound of formula (A) was used

### EXAMPLE 11

Sequential Addition Procedure Using Lithium Amide as the Base (Solid Addition)

Lithium amide procedure to make 2-(2-chloro-4-iodophenylamino)-3,4-difluorobenzoic acid

To an inerted flask fitted with a stir bar was added 8 g (31.6 mmol) 2-chloro-4-iodoaniline, 6 g (34 mmol) 2,3,4-trifluorobenzoic acid and 50 mL acetonitrile. 2.7 g (117 mmol) lithium amide was added at room temperature in portions over 2 days. The reaction was heated to 60° C. for about 1 hour, then cooled to room temperature and quenched by the addition of aqueous hydrochloric acid. After cooling to 0° C. to −15° C., the reaction was filtered and the cake washed with an acetonitrile/water mixture. The wet cake was dried in a vacuum oven resulting in 12.8 g (98%) of 2-(2-chloro-4-iodophenylamino)-3,4-difluorobenzoic acid (CIPFA).

### EXAMPLE 12

Lithium amide procedure to make 2-(2-chloro-4-iodophenylamino)-3,4-difluorobenzoic acid using the sodium salt of 2,3,4-trifluorobenzoic acid

To an inerted flask fitted with a stir bar was added 5 g (20 mmol) 2-chloro-4-iodoaniline, 4.3 g (22 mmol) 2,3,4-trifluorobenzoic acid sodium salt, 2.0 g of lithium amide powder, and 50 mL acetonitrile. The reaction was stirred at room temperature for 16 hours, then 35 mL of aqueous hydrochloric acid was added and the slurry cooled to −5° C. The product was isolated by filtration and dried in a vacuum oven resulting in 7.5 g (93%) of 2-(2-chloro-4-iodophenylamino)-3,4-difluorobenzoic acid (CIPFA).

Alternate Addition Procedures Using Lithium Hydride and/or Lithium Amide

### EXAMPLE 13

Lithium hydride coupling procedure to make 2-(2-chloro-4-iodophenylamino)-3,4-difluorobenzoic acid

To an inerted 22 L three-neck flask fitted with a condenser, mechanical stirrer, thermocouple, and dropping funnel was added 85 g (10.6 mol) lithium hydride, 823 g (3.25 mol) 2-chloro-4-iodoaniline and 6 L acetonitrile. A solution of 630 g (3.58 mol) 2,3,4-trifluorobenzoic acid in 7 L acetonitrile was added causing a temperature rise to about 60° C. The slurry was stirred at 60° C. to 70° C. for 44 hours, after which time a solution is formed. To this was added a mixture of 1.75 L (21 mol) of 37% aqueous hydrochloric acid and 4.5 L of water. The resulting product slurry was cooled to 0° C. to −15° C., and after about 1 hour of stirring, the product was collected by filtration, and the filter cake washed with 8 L of 1:1 acetonitrile/water. The wet cake was dried in a vacuum yielding 1.2 Kg (90%) of 2-(2-chloro-4-iodophenylamino)-3,4-difluorobenzoic acid.

### EXAMPLE 14

Lithium hydride coupling procedure to make 2-(2-chloro-4-iodophenylamino)-3,4-difluorobenzoic acid using the sodium salt of 2,3,4-trifluorobenzoic acid

To an inerted 12 L flask fitted with mechanical stirring was added 580 g (23 mol) 2-chloro-4-iodoaniline, 500 g (2.53 mol) 2,3,4-trifluorobenzoic acid sodium salt, 50 g of lithium hydride powder (30 mesh), and 6 L anhydrous acetonitrile. The reaction was heated to 57° C. and stirred for 16 hours, then cooled to about 40° C., and 3.5 L of 12% aqueous hydrochloric acid added (exotherm to 55° C.). The slurry was cooled to 0° C., and the product collected by filtration. The filter cake was washed with aqueous acetonitrile, and dried in a vacuum oven, resulting in 860 g (91%) of 2-(2-chloro-4-iodophenylamino)-3,4-difluorobenzoic acid.

### EXAMPLE 15

Preparation of Compound (1) Using the Acid Chloride and the HCl Salt of an Amine with Aqueous Sodium Hydroxide

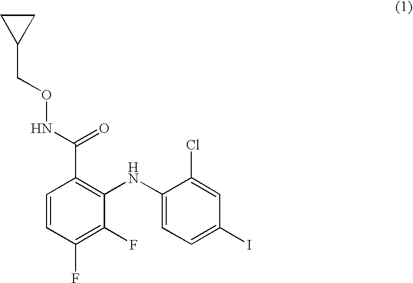

To a 2 L pressure vessel was added CIPFA (70 g, 171 mmol), dimethylformamide (“DMF”) (0.66 mL, 8.6 mmol), and toluene (560 mL). The reaction flask was sealed, and a vacuum of ˜20 inches Hg was applied. Thionyl chloride (15.0 mL, 205 mmol) was added by vacuum addition followed by a toluene line rinse (10 mL). The thick reaction mixture was stirred and heated gradually to 60° C. Over about 4 hours, the reaction mixture changed from an off-white slurry to a yellow solution, and the internal pressure rose from about −20 inches Hg to about 12 psi. The toluene solution was washed with cold water (250 mL), and the organic layer was used directly in the next step.

To a 1000 mL three-neck round bottom flask was added o-cyclopropylmethylhydroxylamine hydrochloride CPMON.HCl (25.4 g, 205 mmol) and 5 M NaOH (164 mL, 820 mmol). The mixture was stirred at room temperature until all the CPMON.HCl was dissolved. To this vigorously stirred solution was added the solution of the CIPFA acid chloride (171 mmol, 0.3 M in toluene) prepared above dropwise while holding the temperature below 35° C. A white slurry formed during the addition. This was stirred for 1 hour at room temperature, and quenched with concentrated HCl solution (35 mL), EtOAc (400 mL), and water (150 mL). This mixture was heated to 45° C. to dissolve the solids, and the lower aqueous layer was removed. The organic layer was washed with water at 40° C. to 45° C. (2×250 mL). After distilling off about 400 mL solvent, the organic layer was allowed to crystallize overnight. The slurry was cooled in an ice/acetone bath for about 2 hours, and then vacuum filtered. The cake was washed with toluene (2×100 mL) and water (100 mL) and dried in a vacuum oven. 71.3 g of white solid remained (87% yield, 99.8% pure by HPLC area %).

### EXAMPLE 16

Preparation of Compound (1) Using Monoprotonation of the Dianion Followed by Reaction with Thionyl Chloride and an Amine Free Base

To a solution of 8 g 2-chloro-4-iodoaniline and 6 g 2,3,4-trifluorobenzoic acid in 50 mL THF was added 2.3 g lithium amide in portions over 2 days. The reaction was stirred at 20° C. to 40° C. for an additional 16 hours, then heated to reflux, then allowed to cool to room temperature.

The above solution of dianion (31 mmol) was cooled to 0° C., and methanesulfonic acid (3.0 g, 31 mmol) was added. The solution was stirred for 30 minutes at 0° C. Thionyl chloride (7.3 g, 62 mmol) was added and the mixture allowed to warm to room temperature and stirred overnight. The reaction mixture was cooled to 0° C., and o-cyclopropylmethylhydroxylamine (5.9 g, 68 mmol) was added followed by triethylamine (9.4 g, 93 mmol). The reaction mixture was allowed to warm to room temperature and stirred overnight. After adding 50 mL of water, the mixture was acidified to pH about 1 and extracted with ethyl acetate. Following three water washes, the organic layer was stripped to dryness, then recrystallized from toluene to give 12.0 g of compound (1) (81%).

### EXAMPLE 17

Preparation of Compound (1) Via an Acid Chloride

To a 250 mL three-neck round bottom flask was added 2-(2-chloro-4-iodophenylamino)-3,4-difluorobenzoic acid (6.0 g, 14.6 mmol) and the flask was purged with N₂. Toluene (70 mL) was added followed by DMF (3 drops) and the thick slurry was heated to 60° C. under a slow stream of N₂. Thionyl chloride (1.6 mL, 22.1 mmol) was added slowly, and the temperature was held at 60° C. for 4 hours. At this point, the reaction mixture became a homogeneous solution. After cooling to about 30° C., the contents of the flask were vacuum distilled to a volume of about 50 mL. Care was taken to ensure that the temperature of the flask was kept below 55° C. The reaction mixture was cooled to −5° C., and to this orange solution was then added o-cyclopropylmethylhydroxylamine (1.5 g, 16.9 mmol) and triethylamine (4.5 g, 44.1 mmol) while holding the temperature below 10° C. Upon complete addition, the contents of the flask were stirred at −5° C. for 30 minutes, and then warmed to room temperature and stirred overnight. The reaction was quenched by the addition of ethyl acetate (40 mL), water (25 mL), and concentrated HCl solution (7 mL). The mixture was heated to 40° C. to 45° C. for 15 minutes, and then stirring was stopped. The lower aqueous layer was removed, and the organic layer was washed twice with water (25 mL) at 40° C. to 45° C. The organic layer was then distilled to a volume of about 50 mL, and allowed to cool slowly to room temperature. After cooling for 2 hours in an ice/acetone bath, the precipitate was vacuum filtered and washed twice with toluene (10 mL) and once with water (10 mL). After drying in a vacuum oven, compound (1) as an off-white solid was obtained (6.2 g, 88%).

### EXAMPLE 18

Preparation of Compound (1) Using N,N-carbonyldiimidazole

To a 50 L glass reactor was added 2.75 Kg 2-(2-chloro-4-iodophenylamino)-3,4-difluorobenzoic acid and 1.25 Kg N,N-carbonyldiimidazole, followed by 18 L anhydrous acetonitrile. After stirring at room temperature for 90 minutes, 0.7 Kg of o-cyclopropylmethylhydroxylamine was added, and the reaction was stirred at room temperature for 16 hours. The slurry was heated to 65° C. to redissolve, then filtered through a scintered glass funnel, and diluted with 3 L hot water. The resulting slurry was cooled to −15° C., and the product was collected by filtration, washed with 14 L of a mixture of acetonitrile and water, and dried in a vacuum oven resulting in 2.72 Kg (86%) of compound (1).

### EXAMPLE 19

Preparation of Compound (1) Using BOP

To a suspension of 2-(2-chloro-4-iodophenylamino)-3,4-difluorobenzoic acid (25.1 g, 61.3 mmol) in THF (300 mL) and CH₂Cl₂ (300 mL) was added O-cyclopropylmethylhydroxylamine hydrochloride (9.1 g, 73.5 mmol), and then the mixture was cooled in ice bath. Diisopropylethylamine (37.4 mL, 0.214 mol) was added, followed by slow addition of benzotriazol-1-yloxy-tris(dimethylamino)-phosphonium hexafluorophosphate (BOP, 32.5 g, 73.54 mmol). The solution was stirred at ambient temperature overnight (20 hours). The mixture was concentrated, and partitioned between tBuOMe and 1N HCl. The organic layer was washed with 1N HCl, brine, sat. solution of NaHCO₃ respectively, and dried (MgSO₄). Crude product was recrystallized from heptane-acetone to give compound (1) as a white solid 26.4 g, 90.2%, mp 177.5-178.5° C.

### EXAMPLE 20

Preparation of Compound (2) Using Lithium Amide Followed by CDI

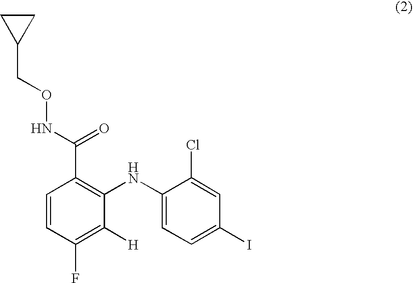

To an inerted flask containing a solution of 24 g (95 mmol) 2-chloro-4-iodoaniline and 15 g (95 mmol) 2,4-difluorobenzoic acid in 150 mL acetonitrile was added 7.6 g (330 mmol) lithium amide in portions over 4 days. The reaction was stirred at room temperature for an additional day, then quenched by the addition of 100 mL aqueous HCl. The resulting slurry was cooled to 5° C., and the product was collected by filtration, washed with a mixture of acetonitrile/water, and dried in a vacuum oven resulting in 29 g (78%) of acid product.

To a glass flask was added 21.2 g 2-(2-chloro-4-iodophenylamino)-4-fluorobenzoic acid and 10 g N,N-carbonyldiimidazole followed by 170 mL anhydrous acetonitrile. After stirring at room temperature for 60 minutes, 8 g of O-cyclopropylmethylhydroxylamine was added, and the reaction was stirred at room temperature for 20 hours. The solution was diluted with 15 mL water, the resulting slurry was cooled to −5° C., and the product was collected by filtration, washed with a mixture of acetonitrile and water, and dried in a vacuum oven resulting in 13.5 g (55%) of compound (2).

### EXAMPLE 21

Preparation of Compound (2) Using Lithium Amide in THF

To a solution of 2,4-difluorobenzoic acid (0.69 g, 4.34 mmol, 1.1 eq.) and 2-chloro-4-iodo-aniline (1.0 g, 3.95 mmol) in dry THF (60 mL), at ambient temperature, was added LiNH₂ (0.32 g, 13.81 mmol, 3.5 eq.). The mixture was stirred at ambient temperature over night (18 hours). THF was rotary evaporated. The residue was dissolved in tBuOMe, washed with 2N HCl, H₂O (2×), brine, and dried over MgSO₄. Evaporation gave brown solid, which was stirred in hexane-CH₂Cl₂ (4:1) for 30 minutes. Solid was filtered, washed with hexane, and dried at 40° C. under vacuum overnight to give 2-(2-chloro-4-iodophenylamino)-4-fluorobenzoic acid, 1.46 g, 85.9%, mp 238-239° C.

To a solution of 2-(2-chloro-4-iodophenylamino)-4-fluorobenzoic acid (1.0 g, 2.55 mmol) in dry THF (40 mL), cooled in ice bath, was added N-methylmorpholine (0.7 mL, 6.38 mmol) followed by diphenylphosphonic chloride (0.78 g, 3.32 mmol). The mixture was stirred for 30 minutes, and O-cyclopropylmethylhydroxylamine (0.31 g, 3.58 mmol) was added. The ice bath was removed, and the mixture was stirred at ambient temperature over night (18 hours). The mixture was concentrated, and tBuOMe was added. The organic solution was washed with saturated solution of NaHCO₃, water respectively, dried over MgSO₄. Crude solid was triturated with hexane-CH₂Cl₂ (4:1) to give compound (2) as an off-white solid, 1.14 g (97%), mp 141-142° C.

### EXAMPLE 22

Preparation of Compound (3) Using Lithium Amide

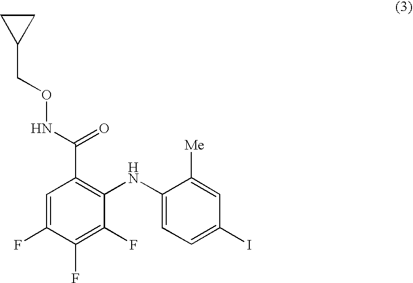

Step (a) In an inerted three-neck round-bottomed flask equipped with a thermometer and powder addition funnel were dissolved 2,3,4,5-tetrafluorobenzoic acid (30.00 g, 154.6 mmol, 1 eq.) and 4-iodo-2-methyl aniline (36.15 g, 154.6 mmol, 1 eq.) in a mixture of THF (220 mL) and acetonitrile (220 mL). The flask was placed in a water bath, and to this solution was added LiNH₂ (11.1 g, 479.2 mmol, 3.1 eq.) over 20 minutes. The temperature of the solution was kept below 30° C. during the addition. After 30 minutes, 1.79 g (0.5 eq.) of LiNH₂ was added in one portion, and the same operation was repeated after another 10 minutes in order to drive the reaction to completion. The dark green mixture was quenched with 1N HCl until pH was acidic, and the mixture was extracted with diethyl ether (3×). The combined organic extracts were washed with brine and dried over MgSO₄. The solvent was removed under vacuum, and the crude solid thus obtained was triturated in CH₂Cl₂ to give 34.33 g (54% yield, mp 206-210° C.) of 2-(4-iodo-2-methylphenylamino)-3,4,5-trifluorobenzoic acid as a bright green solid.

Step (b) To an inerted three-necked, round-bottomed flask, containing a solution of 2-(4-iodo-2-methylphenylamino)-3,4,5-trifluorobenzoic acid (30.00 g, 73.7 mmol, 1 eq) in dry THF (150 mL) was added 4-methylmorpholine (20.3 mL, 184.2 mmol, 2.5 eq). After cooling this mixture to −20° C., a solution of diphenylphosphinic chloride (18.3 mL, 95.8 mmol, 1.3 eq) in dry THF (30 mL), cooled at −20° C., was added via cannula. The resulting mixture was stirred at this temperature for 30 minutes and then a solution of O-cyclopropylmethylhydroxylamine (8.99 g, 103.2 mmol, 1.4 eq) in dry THF (30 mL), cooled at −20° C., was added via cannula. The mixture was stirred at −20° C. for 1.5 hours, then allowed to warm to ambient temperature overnight (20 hours). The reaction mixture was concentrated at reduced pressure to a paste, and the paste was dissolved in ethyl acetate. The organic layer was washed with brine, 1 M KHSO₄ (2×), saturated aqueous NaHCO₃ (2×), brine, and dried over MgSO₄. The solvent was removed under vacuum to give a foamy product that was passed through a plug of silica gel with CH₂Cl₂ as eluent to give 31.68 g (90% yield, mp 137-139° C.) of compound (3) as an off-white solid.

### EXAMPLE 23

Preparation of Compound (4) Using Lithium Hydride

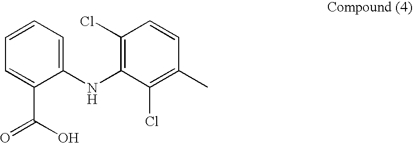

Compound (4)

To a 100 mL flask was added 3 g of 2-fluorobenzoic acid, 4 g of 2,6-dichloro-3-methyl aniline, 0.5 g lithium hydride, and 35 mL diethoxyethane. The mixture was heated to 80° C. for 130 hours, allowed to cool to 45° C., and diluted with 20 mL of 20% aqueous HCl. After cooling to −10° C., the product was collected by filtration, washed with aqueous acetonitrile, and dried in a vacuum oven resulting in 4 g of off-white product 2-(2,6-Dichloro-3-methyl-phenylamino)-benzoic acid, compound (4).

### EXAMPLE 24

Preparation of 2-{4-[2-(3,4-Dichlorophenyl)-ethyl]-phenylamino}-benzoic acid—Compound (5)

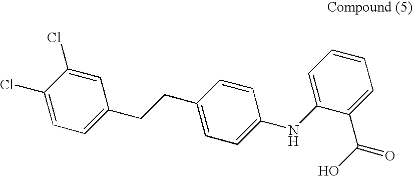

An inerted flask was charged with lithium amide powder (9.4 gm) and 25 mL tetrahydrofuran. To this slurry was added a solution containing 4-[2-(3,4-dichlorophenyl)-ethyl]-benzenamine (22 g), 2-fluorobenzoic acid (11.5 g), and tetrahydrofuran (75 mL). The mixture was then heated to (50-65° C.) for several hours, and monitored (HPLC) for the loss of starting materials. Upon completion, the reaction was quenched with the addition of aqueous HCl, the aqueous layer discarded, and the organic layer washed with additional water. The organic layer was treated with carbon, filtered and the product precipitated by the addition of 150 mL methanol followed by 20 mL water. The thick slurry was cooled to 0° C. overnight and the product collected by filtration, washed with 100 mL of a mixture of 80:20 methanol/water, then dried in a vacuum oven yielding 22.7 g (71.4%) of compound (5) as a pale yellow solid.

### EXAMPLE 25

Preparation of 2-(Indolin-1-yl)benzoic acid

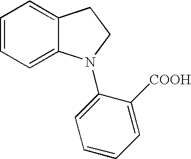

To a 250 mL flask was added indoline (5 g, 42.0 mmol), 2-fluorobenzoic acid (6.2 g, 44.1 mmol), and THF (140 mL). To this solution was added lithium amide (2.0 g, 88.2 mmol) in two portions over 5 minutes. This mixture was heated to 50° C. under nitrogen for about 4 hours, then cooled to room temperature. The reaction was quenched with water (25 mL), concentrated HCl (10 mL), and tBuOMe (25 mL). The aqueous layer was removed, and the organic layer was washed with water (25 mL). Following removal of solvent in vacuo, the resulting yellow solid was dissolved in isopropyl alcohol (“IPA”) (40 mL) at 65° C., and water (48 mL) was added slowly. Following slow cooling to 3° C., the product was filtered and washed with 40% IPA in water (2×10 mL). The off-white solid was dried in vacuum oven at about 50° C. to give 7.9 g (79% yield) of 2-(indolin-1-yl)benzoic acid.

### EXAMPLE 26

Preparation of 2-(Diphenylamino)benzoic acid

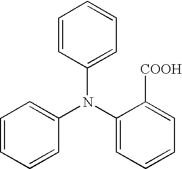

To a 250 mL flask was added diphenylamine (5 g, 29.5 mmol), 2-fluorobenzoic acid (4.3 g, 30.7 mmol), and THF (100 mL). To this solution was added lithium amide (1.4 g, 61.0 mmol) in two portions over 5 minutes. This mixture was heated to 60° C. under nitrogen for about 72 hours, then cooled to room temperature. The reaction was quenched with water (25 mL), concentrated HCl (5 mL), and tBuOMe (25 mL). The aqueous layer was removed, and the organic layer was washed with water (25 mL). Following removal of solvent in vacuo, the product was dissolved in ethyl acetate (100 mL) and the remaining aqueous layer was removed. The solvent was removed in vacuo, and the wet solid was dissolved in IPA (70 mL) at 70° C. Following slow cooling to −10° C., the product was filtered and washed with IPA (10 mL). The bright yellow solid was dried in vacuum oven at about 50° C. to give 5.8 g (68% yield) of 2-(diphenylamino)benzoic acid.

### EXAMPLE 27

Preparation of N-Cyclopropylmethyloxy-2-(4-iodo-2-methylphenylamino)-3,4,5-trifluorobenzamide

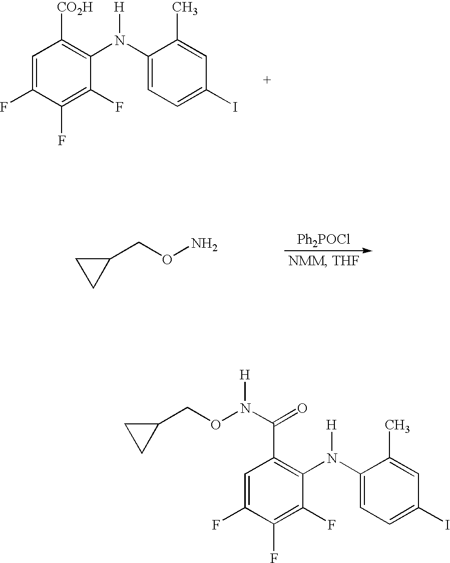

In a three-necked round-bottomed flask equipped with a stirring bar and a low temperature thermometer and under a nitrogen atmosphere was dissolved 2-(4-iodo-2-methylamino)-3,4,5-trifluorobenzoic acid (2.00 g, 4.91 mmol) in dry THF (10 mL). N-methylmorpholine (“NMM,” 1.1 mL, 9.82 mmol) was added, and the contents of the flask were cooled to −20° C. In a second round-bottomed flask equipped with a stirring bar and a low temperature thermometer and under a nitrogen atmosphere was prepared a solution of diphenylphosphinic chloride (“DPPCl”, 1.03 mL, 5.40 mmol) in dry THF (2 mL), cooled to −20° C. and cannulated into the first flask. The mixture was allowed to stir at this temperature for 15 minutes, and then a solution of O-cyclopropylmethylhydroxylamine (0.47 g, 5.40 mmol) in dry THF (2 mL) was added. The mixture was stirred at −20° C. for 1.5 hours and allowed to warm up to ambient temperature overnight. The solvent was removed under vacuum, and the residue taken up in ethyl acetate (50 mL). The organic phase was washed with brine (20 mL), 1 M KHSO₄ aqueous solution (20 mL), saturated NaHCO₃ aqueous solution (2×20 mL), brine (20 mL), and dried over MgSO₄. The solvent was removed under vacuum, and the residue chromatographed on silica gel (eluent: CH₂Cl₂) to give 2.15 g (92%) of N-cyclopropylmethyloxy-2-(4-iodo-2-methylphenylamino)-3,4,5-trifluorobenzamide as a yellow solid.

### EXAMPLE 28

2-(2-Fluoro-4-iodophenylamino)-3,4-difluorobenzoic acid

In an inerted three-necked round-bottomed flask equipped with mechanical stirrer and reflux condenser were dissolved 2,3,4-trifluorobenzoic acid (37.57 g, 213.4 mmol) and 2-fluoro-4-iodoaniline (50.57 g, 213.4 mmol) in dry acetonitrile (740 mL). Lithium amide (19.59 g, 853.46 mmol) was then added in small portions over 15 minutes, and the resulting suspension was refluxed for 1 hour, during which time the color went from gray-pink to dark green. The flask was placed in an ice water bath, and the reaction was quenched with concentrated HCl to pH 1. Water (2 L) was then added, and the resulting solid was filtered, washed with water (2×500 mL), and dried in a vacuum oven at 50° C. for 18 hours. The solid thus obtained was triturated with CH₂Cl₂ (500 mL), filtered, washed with fresh CH₂Cl₂ (2×100 mL), and dried in a vacuum oven at 50° C. for 24 hours to give 55.8 g (66%) of 2-(2-fluoro-4-iodophenylamino)-3,4-difluorobenzoic acid as a light brown solid; mp 199-201° C.

The results for Examples 11-28 expressed as percent yields are found in the Examples themselves.

Three further embodiments of Method A, Method B, or Method C, namely Method A1, Methods B1 and B2, and Method C1, respectively, were used in the preparation of Examples 25-39 and Preparations 3-8. In Method A1, a “two-pot procedure”, in a first flask, a base (1 mol equivalent) was added to a solution of a compound of formula (B), wherein Z is COOH (1 mol equivalent), in an aprotic solvent such as, for example, tetrahydrofuran (“THF”) at about −78° C. In a second flask, base (2 mol equivalents) was added to a solution of a compound of formula (A) (1 mol equivalent) in an aprotic solvent such as, for example, THF at about −78° C. The contents of the first flask were transferred into the second flask, and the resulting mixture was allowed to warm, or warmed, overnight to a temperature such as, for example, ambient temperature to allow the reaction to progress satisfactorily before the reaction was worked up.

In Method B1, a “one-pot procedure”, both a compound of formula (B), wherein Z is COOH (1 mol equivalent), and a compound of formula (A) (1 mol equivalent) were dissolved in an aprotic solvent such as, for example, THF, the mixture was cooled to about −78° C., and a base was added. The mixture was allowed to warm, or warmed, overnight to a temperature such as, for example, ambient temperature to allow the reaction to progress satisfactorily before the reaction was worked up.

In Method B2, both a compound of formula (B), wherein Z is COOH (1 mol equivalent), and a compound of formula (A) (1 mol equivalent) were dissolved in an aprotic solvent such as, for example, THF, the mixture was cooled to about −20° C. to about 0 ° C., and a base (3 mol equivalents) was added. The mixture was warmed overnight to a temperature such as, for example, 40° C.-50° C. to allow the reaction to progress satisfactorily before the reaction was worked up.

In Method C1, which is also a two-pot procedure, in a first flask, a solution of a compound of formula (B), wherein Z is COOH (1 mol equivalent), in an aprotic solvent such as, for example, THF at about −78° C. was made. In a second flask, base (3 mol equivalents) was added to a solution of a compound of formula (A) (1 mol equivalent) in an aprotic solvent such as, for example, THF at about −78° C. The contents of the first flask were transferred into the second flask, and the resulting mixture was allowed to warm, or warmed, overnight to a temperature such as, for example, ambient temperature to allow the reaction to progress satisfactorily before the reaction was worked up.

Additional Examples 29 and 30, indicated by their Example number in the column labelled “Ex. No.”, are shown below in Table 3. Results are shown as percent yield of a compound of Formula I in the column labelled “Yield (%)”. The reactants are a compound of formula (A) and a compound of formula (B), which are shown in the columns labelled “(A)” and “(B)”, respectively. The base and method employed are shown in the columns labelled “Base” and “Method”, respectively. Three (3) mole-equivalents of base were employed unless otherwise indicated.

TABLE 3

| Ex. | | | | | Yield |
|---|---|---|---|---|---|
| No. | (B) | (A) | Base | Method | (%) |
| 29 30 | 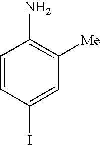 | 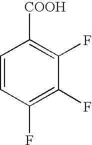 | LDA NaH | B1 B1 | 28 ND(f) |

### EXAMPLE 31

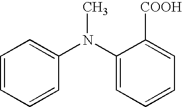

2-(N-Methyl-N-phenylamino)-benzoic acid

To a 250 mL flask was added N-methylaniline (3.75g, 35.0 mmol), 2-fluorobenzoic acid (5.1 g, 36.8 mmol), and THF (115 mL). To this solution was added lithium amide (1.7 g, 73.5 mmol) in two portions over 5 minutes. This mixture was heated to 50° C. under nitrogen for about 3.5 hours, then cooled to room temperature. The reaction was quenched with water (25 mL), conc. HCl (10 mL), and MTBE (25 mL). The aqueous layer was removed, and the organic layer was washed with water (25 mL). Following removal of solvent in vacuo, the product was dissolved in isopropyl alcohol (“IPA”) (25 mL) at 70° C. Water (10 mL) was added, and following slow cooling to −10° C., the resulting product was filtered and washed with 4:6 IPA:water (10 mL). The resulting yellow solid was dried in a vacuum oven at about 50° C. to give 6.9 g (87% yield) of 2-(N-methyl-N-phenylamino)-benzoic acid; mp 105-106° C.

### EXAMPLE 32

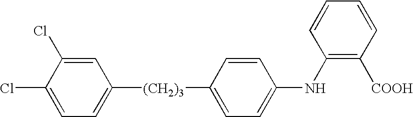

2-{4-[3-(3,4-Dichlorophenyl)-propyl]-phenylamino}-benzoic acid

To an inerted flask containing 7.1 g (25 mmol) of 4-[3-(3,4-dichlorophenyl)-propyl]-aniline, 3.6 g (26 mmol) of 2-fluorobenzoic acid, and 70 mL THF was added 2.0 g (87 mmol) of lithium amide powder. The reaction was heated at 55° C. for 6 hours, then cooled to room temperature and quenched by the addition of water and aqueous hydrochloric acid. The layers were separated, the top layer concentrated in vacuo, and the solid crystallized from acetone and water. The solid was filtered off, and the filter cake was washed with an acetone/water mixture and dried in a vacuum oven to give 7 g (70% yield) of 2-{4-[3-(3,4-dichlorophenyl)-propyl]-phenylamino}-benzoic acid as a light yellow solid; mp 133° C.

Additional Examples 33-35, indicated by their Example number in the column labelled “Ex. No.”, are shown below in Table 4. Results are shown as percent yield of a compound of Formula I in the column labelled “Yield (%)”. The reactants are a compound of formula (A) and a compound of formula (B), which are shown in the columns labelled “(A)” and “(B)”, respectively. The base and method employed are shown in the columns labelled “Base” and “Method”, respectively. Three (3) mole-equivalents of base were employed unless otherwise indicated.

TABLE 4

| P. | | | | | Yield |
|---|---|---|---|---|---|
| No. | (A) | (B) | Base | Method | (%) |
| 33 | 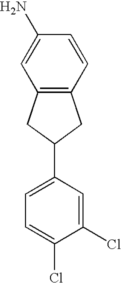 | 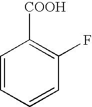 | LiHMDS | B2 | 76^{c} |
| 34 | 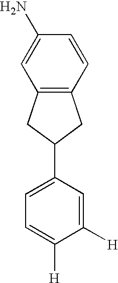 | 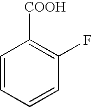 | LiHMDS | B2 | 76 |
| 35 | 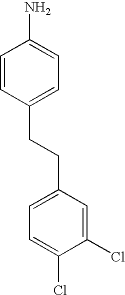 | 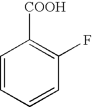 | LIHMDS | C1 | 99^{d} |

The data in Tables 1-4 were collected from non-optimized experiments. The amount of product would be expected to increase if reaction conditions were optimized. An entry of “ND” indicates that product was not detected. This is not to say that product could not be obtained using the process of the present invention. Rather, such an entry indicates that under the specific reaction conditions used, the amount of product was below the detection limits or was simply not determined.

It is shown by the above examples that the sequential addition method of Example 1 and use of lithium amide of Examples 11 and 12 above surprisingly improves the yield of the invention process.

Although the method of the present invention typically provides high selectivity for ortho substitution over para substitution, the preparation of 2-(4-iodo-2-methylphenylamino)-3,4,5-trifluorobenzoic acid from 4-iodo-2-methylaniline and 2,3,4,5-tetrafluorobenzoic acid in THF typically provided mixtures of the desired compound and the para-substituted regioisomer, namely 4-(4-iodo-2-methylphenylamino)-2,3,5-trifluorobenzoic acid. These mixtures were hard to purify. As shown in Example 22, Step (a), use of a mixture of about 1 part THF and about 1 part acetonitrile provides the desired ortho-substituted product without contamination by the corresponding para-substituted regioisomer.

In the process of the present invention, Group I metal cation hydride and Group I metal cation amide bases are preferred over Group I metal cation and Group II metal cation bis(trialkylsilyl)amides for the preparation of a compound of Formula I, wherein the compound of Formula I is as defined above except Z is COOH or COOM, wherein M is a Group I metal cation or hemi Group II metal cation. Bases such as lithium hexamethyldisilazide must be preformed for best results, as the bases degrade slowly over time, and the commercially available materials are usually impure. More importantly, bases such as lithium hexamethyldisilazide should be added sequentially to avoid formation of a reactive benzyne intermediate. Said benzyne intermediate has been observed when, for example, 3 mol equivalents of LiHMDS is added all at once to a reaction of the invention process.

Group I metal cation hydride and Group I metal cation amide bases are solids, which can be added to the reaction all at once while still providing best results. Since the bases are solids, the amount of base in contact with reactants is controlled by the rate of dissolution of the base and/or limited surface area contact of the reactants with the solid particles of the base. Further, the Group I metal cation hydrides and the Group I metal cation amides do not have to be preformed before use. The advantages of the Group I metal cation hydride and Group I metal cation amide bases over Group I metal cation and Group II metal cation bis(trialkylsilyl)amides in the preparation of a compound of Formula I, wherein the compound of Formula I is as defined above except Z is COOH or COOM, wherein M is a Group I metal cation or hemi Group II metal cation, are important for satisfactory commercial scale production.

Another advantage of the process of the instant invention lies in the discovery of superior carboxylic acid activating reagents for the coupling of a compound of Formula I as defined above except wherein Z is COOH or COOM, wherein M is a Group I metal cation or hemi Group II metal cation, with a compound of formula II, III, or IV, each as defined above, to give a product which is a compound of Formula I, wherein Z is COOR¹⁵, —C(O)N(R¹⁶)R¹⁷, or —C(O)N(R¹⁸)OR¹⁹, wherein R¹⁵, R¹⁶, R¹⁷, R¹⁸, and R¹⁹ are as defined above. Coupling using PyBOP typically provided products in low yields, and purification of the resulting product was difficult. The present invention process employs carboxylic acid activating reagents such as thionyl chloride, DPPCl, or EDC, in particular. These reagents provide product in higher yields. Further, the products are easier to purify typically. Still further, the cost of the carboxylic acid activating reagents used in the instant invention is usually lower than the cost of PyBOP. These advantages are important for commercial scale production.

What is claimed is:

1. A process of synthesizing a compound of Formula I 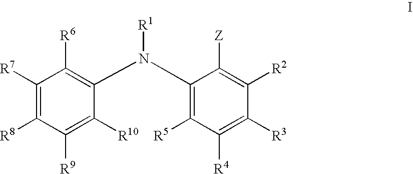
or a pharmaceutically acceptable salt thereof,
wherein:
R¹ is hydrogen, alkyl, alkoxy, or aryl;
R², R³, R⁴, R⁵, R⁶, R⁷, R⁸, R⁹, and R¹⁰ are each independently selected from:
hydrogen,
halo,
alkyl,
aryl,
a heterocyclic group,
haloalkyl,
alkoxy,
nitro,
CN,
—(O)ₘ—(CH₂)ₙ—R¹¹, or
—[N(H)]ₘ—(CH₂)ₙ—R¹¹, wherein m, n, and R¹¹ are as defined below,
or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms,
or R¹ and R⁶ may be taken together with the nitrogen atom to which R¹ is attached, the carbon atom to which R⁶ is attached, and the carbon atom contiguous to said nitrogen atom to which R¹ is attached and said carbon atom to which R⁶ is attached, to form a 5-membered or 6-membered, aromatic or dihydro-aromatic ring having carbon atoms and 1 or 2 nitrogen atoms;
R¹¹ is hydrogen, hydroxy, —CO₂H, or N(R¹²)R¹³,
R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;
m is an integer of 0 or 1;
n is an integer selected from 0, 1, 2, 3, 4; and
Z is COOH, COOM, COOR15, —C(O)R¹⁵, —C(O)N(R¹⁶)R¹⁷, —C(O)N(R¹⁸)OR¹⁹, NO₂, or CN, wherein
M is a Group I metal cation or a hemi Group II metal cation,
R¹⁵ is alkyl, alkenyl, alkynyl, aryl, or a heterocyclic group, and
R¹⁶, R¹⁷, R¹⁸, and R¹⁹ are each independently selected from hydrogen, alkyl, alkenyl, phenyl, and benzyl, or
R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;
comprising reacting a compound of Formula (A) 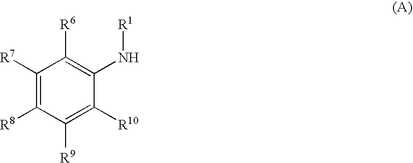
wherein R¹, R⁶, R⁷, R⁸, R⁹, and R¹⁰ are as defined above, with a compound of Formula (B) 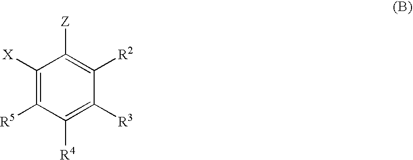
wherein Z, R², R³, R⁴, and R⁵ are as defined above, and X is halo or O-LG, wherein LG is SO₂R²⁰ or P(═O)(OR²⁰)₂, wherein R²⁰ is alkyl or aryl, optionally in a solvent, and in the presence of from about 1 mol equivalent to about 10 mol equivalents of a base, wherein the base is selected from:
a Group I metal cation hydride or a Group 2 metal cation hydride, including lithium hydride, sodium hydride, potassium hydride, and calcium hydride,
a Group I metal cation dialkylamide or a Group 2 metal cation dialkylamide, including lithium diisopropylamide,
a Group I metal cation amide or a Group 2 metal cation amide, including lithium amide, sodium amide, potassium amide, and
a Group I metal cation alkoxide or a Group 2 metal cation alkoxide, including sodium ethoxide, potassium tert-butoxide, and magnesium ethoxide, for a time, and at a temperature, sufficient to yield a compound of Formula I.

2. The process according to claim 1, wherein the base is selected from:
lithium diisopropylamide, lithium hydride, sodium hydride, potassium hydride, lithium amide, sodium amide, potassium amide, sodium methoxide, sodium ethoxide, and potassium tert-butoxide.

3. The process according to claim 1, wherein the base is selected from lithium hydride, sodium hydride, and potassium hydride.

4. The process according to claim 1, wherein the base is lithium hydride.

5. The process according to claim 1, wherein the base is selected from lithium amide, sodium amide, and potassium amide.

6. The process according to claim 1, wherein the base is lithium amide.

7. The process according to claim 1, wherein the base is lithium diisopropylamide.

8. The process according to claim 1, wherein the base is selected from sodium methoxide, sodium ethoxide, and potassium tert-butoxide.

9. The process according to claim 1, wherein from 1 to 5 mol equivalents of base are employed initially, and optionally from 0.5 to 4 additional mol equivalents of base are added to the reaction after a time, wherein said 0.5 to 4 additional mol equivalents of base are added in one portion or are added sequentially in unequal or equal portions at unequal or equal time intervals.

10. The process according to claim 9, wherein said 0.5 to 4 additional mol equivalents of base are added sequentially to the reaction in unequal portions of decreasing size.

11. The process according to claim 10, wherein in the compound of formula (B), Z is COOH and 2 mol equivalents of base are employed initially or Z is COOM and 1 mol equivalent of base is employed initially, and said 0.5 to 4 additional mol equivalents of base are added sequentially to the reaction in unequal portions of decreasing size as follows: about 0.5 mol equivalents, followed by about 0.25 mol equivalents, followed by about 0.13 mol equivalents, followed by about 0.06 mol equivalents, optionally followed by about 0.03 mol equivalents, followed by about 0.015 mol equivalents.

12. The process according to claim 1, wherein R¹ is hydrogen.

13. The process according to claim 1, wherein X is fluoro.

14. The process according to Claim l, wherein X is O-LG, wherein LG is SO₂CF₃, or P(═O)(OCH₂CH₃)₂.

15. The process according to claim 1, wherein R², R³, R⁴, and R⁵ are each independently selected from hydrogen, alkoxy, fluoro, chloro, bromo, and iodo.

16. The process according to claim 1, wherein R⁶, R⁷, R⁸, R⁹, and R¹⁰ are each independently selected from hydrogen, alkyl, fluoro, chloro, bromo, and iodo.

17. The process according to claim 1, wherein Z is COOH or COOM.

18. The process according to claim 1, wherein R¹ is hydrogen, X is fluoro, R², R³, R⁴, and R⁵ are each independently selected from hydrogen, alkoxy, fluoro, chloro, bromo, and iodo, R⁶, R⁷, R⁸, R⁹, and R¹⁰ are each independently selected from hydrogen, methyl, fluoro, chloro, bromo, and iodo, and Z is COOH or COOM.

19. The process according to claim 1, wherein a solvent is present and the solvent comprises acetonitrile, tetrahydrofuran, 1,2-diethoxyethane, 2,2-dimethoxypropane, 1,2-dimethoxypropane, diethylether, dioxane, or methyl tert-butylether.

20. The process according to claim 1, wherein a solvent is present and the solvent comprises tetrahydrofuran or acetonitrile.

21. The process according to claim 1, wherein a solvent is present and the solvent comprises a mixture of from about 1 part by volume of acetonitrile and about 1 part by volume of tetrahydrofuran to about 5 parts by volume of acetonitrile and about 1 part by volume of tetrahydrofuran.

22. The process according to claim 1, wherein when the base is added, the reaction mixture is at a temperature of from −78° C. to 150° C.

23. The process according to claim 1, wherein the compound of Formula I is a 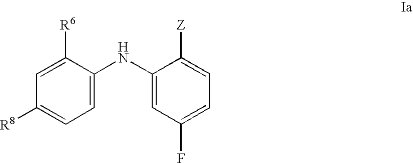
compound of Formula Ia
or a pharmaceutically acceptable salt thereof,
wherein R⁶ is halo or methyl, R⁸ is bromo or iodo, and Z is COOH, COOM, COOR¹⁵, —C(O)R¹⁵, —C(O)N(R¹⁶)R¹⁷, —C(O)N(R¹⁸)OR¹⁹, NO₂, or CN, wherein
M is a Group I metal cation or a hemi Group II metal cation,
R¹⁵ is alkyl, alkenyl, alkynyl, aryl, or a heterocyclic group, and
R¹⁶, R¹⁷, R¹⁸, and R¹⁹ are each independently selected from hydrogen, alkyl, alkenyl, phenyl, and benzyl, or
R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl.

24. The process according to claim 1, wherein the compound of Formula I is a compound of Formula Ib 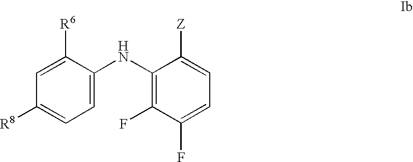
or a pharmaceutically acceptable salt thereof,
wherein R⁶ is halo or methyl, R⁸ is bromo or iodo, and Z is COOH, COOM, COOR¹⁵, —C(O)R¹⁵, —C(O)N(R¹⁶)R¹⁷, —C(O)N(R¹⁸)OR¹⁹, NO₂, or CN, wherein
M is a Group I metal cation or a hemi Group II metal cation,
R¹⁵ is alkyl alkenyl, alkynyl, aryl, or a heterocyclic group, and
R¹⁶, R¹⁷, R¹⁸, and R¹⁹ are each independently selected from hydrogen, alkyl, alkenyl, phenyl, and benzyl, or
R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl.

25. The process according to claim 1, wherein the compound of Formula I is a compound of Formula Ic1 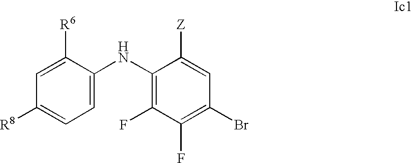
or a pharmaceutically acceptable salt thereof,
wherein R⁶ is halo or methyl, R⁸ is bromo or iodo, and Z is COOH, COOM, COOR¹⁵, —C(O)R¹⁵, —C(O)N(R¹⁶)R¹⁷, —C(O)N(R¹⁸)OR¹⁹, NO₂, or CN, wherein
M is a Group I metal cation or a hemi Group II metal cation,
R¹⁵ is alkyl, alkenyl, alkynyl, aryl, or a heterocyclic group, and
R¹⁶, R¹⁷, R¹⁸, and R¹⁹ are each independently selected from hydrogen, alkyl, alkenyl, phenyl, and benzyl, or
R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl.

26. The process according to claim 1, wherein the compound of Formula I is a compound of Formula Ic2 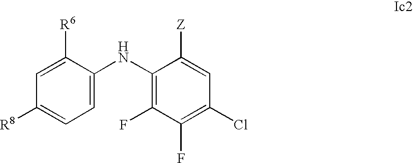
or a pharmaceutically acceptable salt thereof,
wherein R⁶ is halo or methyl, R⁸ is bromo or iodo, and Z is COOH, COOM, COOR¹⁵, —C(O)R¹⁵, —C(O)N(R¹⁶)R¹⁷, —C(O)N(R¹⁸)OR¹⁹, NO₂, or CN, wherein
M is a Group I metal cation or a hemi Group II metal cation,
R¹⁵ is alkyl, alkenyl, alkynyl, aryl, or a heterocyclic group, and
R¹⁶, R¹⁷, R¹⁸, and R¹⁹ are each independently selected from hydrogen, alkyl, alkenyl, phenyl, and benzyl, or
R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl.

27. The process according to claim 1, wherein the compound of Formula I is a compound of Formula Id 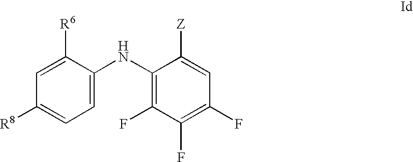
or a pharmaceutically acceptable salt thereof,
wherein R⁶ is halo or methyl, R⁸ is bromo or iodo, and Z is COOH, COOM, COOR¹⁵, —C(O)R¹⁵, —C(O)N(R¹⁶)R¹⁷, —C(O)N(R¹⁸)OR¹⁹, NO₂, or CN, wherein
M is a Group I metal cation or a hemi Group II metal cation,
R¹⁵ is alkyl, alkenyl, alkynyl, aryl, or a heterocyclic group, and
R¹⁶, R¹⁷, R¹⁸, and R¹⁹ are each independently selected from hydrogen, alkyl, alkenyl, phenyl, and benzyl, or
R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl.

28. The process according to claim 1, wherein the compound of Formula I is a compound of formula 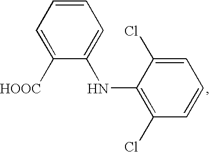
or a pharmaceutically acceptable salt thereof.

29. The process according to claim 1, wherein the compound of Formula I is a compound of formula 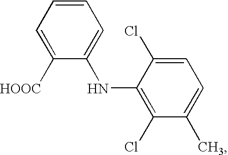
or a pharmaceutically acceptable salt thereof.

30. The process according to claim 1, wherein the compound of Formula I is a compound of formula 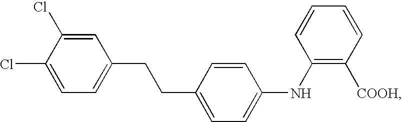
or a pharmaceutically acceptable salt thereof.

31. The process according to claim 1 wherein the compound of Formula I is a compound of formula 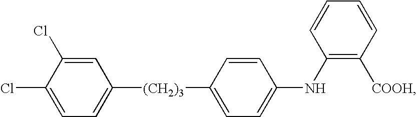
or a pharmaceutically acceptable salt thereof.

32. The process according to claim 1, wherein the compound of Formula I is a compound of formula 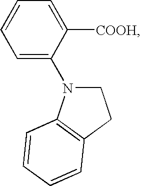
or a pharmaceutically acceptable salt thereof.

33. The process according to claim 1, wherein the compound of Formula I is a compound of formula 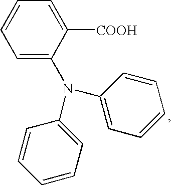
or a pharmaceutically acceptable salt thereof.

34. The process according to claim 1, wherein the compound of Formula I is a compound of formula 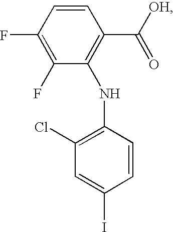
or a pharmaceutically acceptable salt thereof.

35. The process according to claim 1, wherein the compound of Formula I is a compound of formula 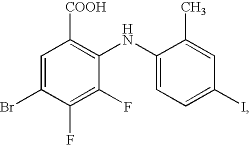
or a pharmaceutically acceptable salt thereof.

36. The process according to claim 1, wherein the compound of Formula I is a compound of formula 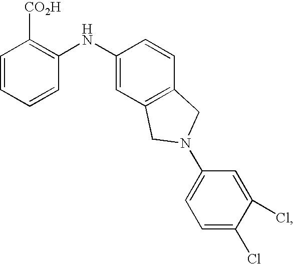
or a pharmaceutically acceptable salt thereof.

37. The process according to claim 1, wherein the compound of Formula I is a compound of formula 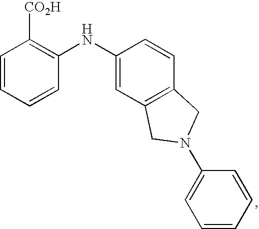
or a pharmaceutically acceptable salt thereof.

38. A process of synthesizing a compound of Formula I 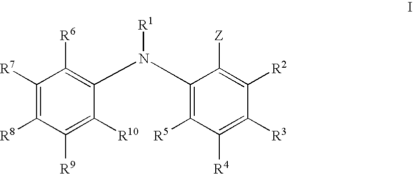
or a pharmaceutically acceptable salt thereof,
wherein:
R¹ is hydrogen, alkyl, alkoxy, or aryl;
R², R³, R⁴, R⁵, R⁶, R⁷, R⁸, R⁹, and R¹⁰ are each independently selected from:
hydrogen,
halo,
alkyl,
aryl,
a heterocyclic group,
haloalkyl,
alkoxy,
nitro,
CN,
—(O)ₘ—(CH₂)ₙ—R¹¹, or
—[N(H)]ₘ—(CH₂)ₙ—R¹¹, wherein m, n, and R¹¹ are as defined below,
or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms,
or R¹ and R⁶ may be taken together with the nitrogen atom to which R¹ is attached, the carbon atom to which R⁶ is attached, and the carbon atom contiguous to said nitrogen atom to which R¹ is attached and said carbon atom to which R⁶ is attached, to form a 5-membered or 6-membered, aromatic or dihydro-aromatic ring having carbon atoms and 1 or 2 nitrogen atoms;
R¹¹ is hydrogen, hydroxy, —CO₂H, or N(R¹²)R¹³,
R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;
m is an integer of 0 or 1;
n is an integer selected from 0, 1, 2, 3, 4; and
Z is COOH, COOM, COOR¹⁵, —C(O)R¹⁵, —C(O)N(R¹⁶)R¹⁷, —C(O)N(R¹⁸)OR¹⁹, NO₂, or CN, wherein M is a Group I, or a hemi Group II, metal cation, R¹⁵ is alkyl, alkenyl, alkynyl, aryl, or a heterocyclic group, and R¹⁶, R¹⁷, R¹⁸, and R¹⁹ are each independently selected from hydrogen, alkyl, alkenyl, phenyl, and benzyl, or
R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;
comprising reacting a compound of Formula (A) 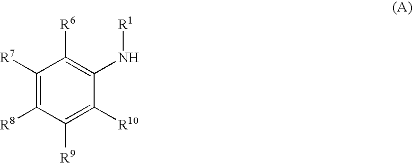
wherein R¹ and R⁶-R¹⁰ are as defined above, with a compound of Formula (B) 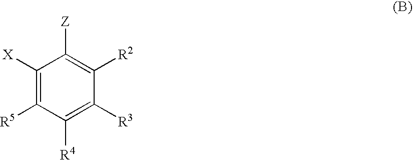
wherein Z is COOR¹⁵, —C(O)R¹⁵, —C(O)N(R¹⁶)R¹⁷, —C(O)N(R¹⁸)OR¹⁹, NO₂, or CN, and R²-R⁵ and R¹⁵-R¹⁹ are as defined above, and X is halo or O-LG, wherein LG is SO₂R²⁰ or P(═O)(OR²⁰)₂, wherein R²⁰ is alkyl or aryl, optionally in a solvent, and in the presence of from about 1 mol equivalent to about 10 mol equivalents of a base, wherein the base is selected from:
a Group I metal cation bis(trialkylsilyl)amide or a Group 2 metal cation bis(trialkylsilyl)amide, including lithium bis(trimethylsilyl)amide, sodium bis(trimethylsilyl)amide, or potassium bis(trimethylsilyl)amide, for a time, and at a temperature, sufficient to yield a compound of Formula I.

39. The process according to claim 38, wherein the base is selected from:
lithium bis(trimethylsilyl)amide, sodium bis(trimethylsilyl)amide, and potassium bis(trimethylsilyl)amide.

40. The process according to claim 38, wherein the base is lithium bis(trimethylsilyl)amide.

41. The process according to claim 38, wherein from 1 to 5 mol equivalents of base are employed initially, and optionally from 0.5 to 4 additional mol equivalents of base are added to the reaction after a time, wherein said 0.5 to 4 additional mol equivalents of base are added in one portion or are added sequentially in unequal or equal portions at unequal or equal time intervals.

42. The process according to claim 41, wherein said 0.5 to 4 additional mol equivalents of base are added sequentially to the reaction in unequal portions of decreasing size.

43. The process according to claim 38, wherein R¹ is hydrogen.

44. The process according to claim 38, wherein X is fluoro.

45. The process according to claim 38, wherein X is O-LG, wherein LG is SO₂CF₃, or P(═O)(OCH₂CH₃)₂.

46. The process according to claim 38, wherein R², R³, R⁴, and R⁵ are each independently selected from hydrogen, alkoxy, fluoro, chloro, bromo, and iodo.

47. The process according to claim 38, wherein R⁶, R⁷, R⁸, R⁹, and R¹⁰ are each independently selected from hydrogen, alkyl, fluoro, chloro, bromo, and iodo.

48. The process according to claim 38, wherein Z is —C(O)N(R¹⁸)OR¹⁹, wherein R¹⁸ and R¹⁹ are each independently selected from hydrogen, alkyl, alkenyl, phenyl, and benzyl.

49. The process according to claim 38, wherein R¹ is hydrogen, X is fluoro, R², R³, R⁴, and R⁵ are each independently selected from hydrogen, alkoxy, fluoro, chloro, bromo, and iodo, R⁶, R⁷, R⁸, R⁹, and R¹⁰ are each independently selected from hydrogen, methyl, fluoro, chloro, bromo, and iodo, and Z is —C(O)N(R¹⁸)OR¹⁹, wherein R¹⁸ and R¹⁹ are each independently selected from hydrogen, alkyl, alkenyl, phenyl, and benzyl.

50. The process according to claim 38, wherein a solvent is present and the solvent comprises acetonitrile, tetrahydrofuran, 1,2-diethoxyethane, 2,2-dimethoxypropane, 1,2-dimethoxypropane, diethylether, dioxane, or methyl tert-butylether.

51. The process according to claim 38, wherein a solvent is present and the solvent comprises tetrahydrofuran or acetonitrile.

52. The process according to claim 38, wherein a solvent is present and the solvent comprises a mixture of from about 1 part by volume of acetonitrile and about 1 part by volume of tetrahydrofuran to about 5 parts by volume of acetonitrile and about 1 part by volume of tetrahydrofuran.

53. The process according to claim 38, wherein when the base is added, the reaction mixture is at a temperature of from −78° C. to 150° C.

54. The process according to claim 38, wherein the compound of Formula I is 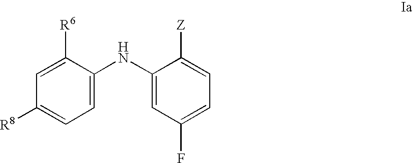
compound of Formula Ia
or a pharmaceutically acceptable salt thereof,
wherein R⁶ is halo or methyl, R⁸ is bromo or iodo, and Z is COOR¹⁵, —C(O)R¹⁵, —C(O)N(R¹⁶)R¹⁷, —C(O)N(R¹⁸)OR¹⁹, NO₂, or CN, wherein
R¹⁵ is alkyl, alkenyl, alkynyl, aryl, or a heterocyclic group, and
R¹⁶, R¹⁷, R¹⁸, and R¹⁹ are each independently selected from hydrogen, alkyl, alkenyl, phenyl, and benzyl, or
R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl.

55. The process according to claim 38, wherein the compound of Formula I is a compound of Formula Ib 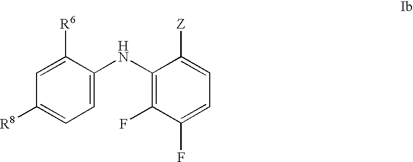
or a pharmaceutically acceptable salt thereof,
wherein R⁶ is halo or methyl, R⁸ is bromo or iodo, and Z is COOR¹⁵, —C(O)R¹⁵, —C(O)N(R¹⁶)R¹⁷, —C(O)N(R¹⁸)OR¹⁹, NO₂, or CN, wherein
R¹⁵ is alkyl, alkenyl, alkynyl, aryl, or a heterocyclic group, and
R¹⁶, R¹⁷, R¹⁸, and R¹⁹ are each independently selected from hydrogen, alkyl, alkenyl, phenyl, and benzyl, or
R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl.

56. The process according to claim 38, wherein the compound of Formula I is a compound of Formula Ic1 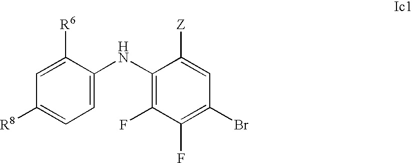
or a pharmaceutically acceptable salt thereof,
wherein R⁶ is halo or methyl, R⁸ is bromo or iodo, and Z is COOR¹⁵, —C(O)R¹⁵, —C(O)N(R¹⁶)R¹⁷, —C(O)N(R¹⁸)OR¹⁹, NO₂, or CN, wherein
R¹⁵ is alkyl, alkenyl, alkynyl, aryl, or a heterocyclic group, and
R¹⁶, R¹⁷, R¹⁸, and R¹⁹ are each independently selected from hydrogen, alkyl, alkenyl, phenyl, and benzyl, or
R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl.

57. The process according to claim 38, wherein the compound of Formula I is a compound of Formula Ic2 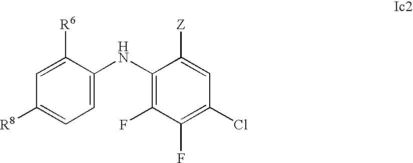
or a pharmaceutically acceptable salt thereof,
wherein R⁶ is halo or methyl, R⁸ is bromo or iodo, and Z is COOR¹⁵, —C(O)R¹⁵, —C(O)N(R¹⁶)R¹⁷, —C(O)N(R¹⁸)OR¹⁹, NO₂, or CN, wherein
R¹⁵ is alkyl, alkenyl, alkynyl, aryl, or a heterocyclic group, and
R¹⁶, R¹⁷, R¹⁸, and R¹⁹ are each independently selected from hydrogen, alkyl, alkenyl, phenyl, and benzyl, or
R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl.

58. The process according to claim 38, wherein the compound of Formula I is a compound of Formula Id 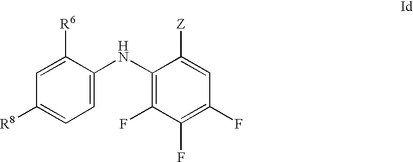
or a pharmaceutically acceptable salt thereof,
wherein R⁶ is halo or methyl, R⁸ is bromo or iodo, and Z is COOR¹⁵, —C(O)R¹⁵, —C(O)N(R¹⁶)R¹⁷, —C(O)N(R¹⁸)OR¹⁹, NO₂, or CN, wherein
R¹⁵ is alkyl, alkenyl, alkynyl, aryl, or a heterocyclic group, and
R¹⁶, R¹⁷, R¹⁸, and R¹⁹ are each independently selected from hydrogen, alkyl, alkenyl, phenyl, and benzyl, or
R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl.

59. The process according to claim 38 wherein the compound of Formula I is a compound of formula 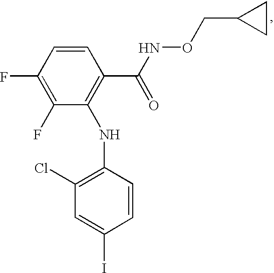
or a pharmaceutically acceptable salt thereof.

60. The process according to claim 38, further comprising hydrolyzing the compound of Formula I wherein Z is COOR¹⁵, wherein R¹⁵ is alkyl, alkenyl, alkynyl, aryl, or a heterocyclic group, to provide the compound of Formula Id2 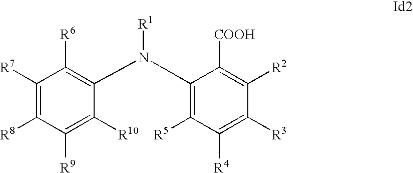
or a pharmaceutically acceptable salt thereof,
wherein:
R¹ is hydrogen, alkyl, alkoxy, or aryl;
R², R³, R⁴, R⁵, R⁶, R⁷, R⁸, R⁹, and R¹⁰ are each independently selected from:
hydrogen,
halo,
alkyl,
aryl,
a heterocyclic group,
haloalkyl,
alkoxy,
nitro,
CN,
—(O)ₘ—(CH₂)ₙ—R¹¹, or
—[N(H)]ₘ—(CH₂)ₙ—R¹¹, wherein m, n, and R¹¹ are as defined below,
or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms,
or R¹ and R⁶ may be taken together with the nitrogen atom to which R¹ is attached, the carbon atom to which R⁶ is attached, and the carbon atom contiguous to said nitrogen atom to which R¹ is attached and said carbon atom to which R⁶ is attached, to form a 5-membered or 6-membered, aromatic or dihydro-aromatic ring having carbon atoms and 1 or 2 nitrogen atoms;
R¹¹ is hydrogen, hydroxy, —CO₂H, or N(R¹²)R¹³,
R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;
m is an integer of 0 or 1; and
n is an integer selected from 0, 1, 2, 3, 4.

61. The process according to claim 60, wherein the compound of Formula Id2 is a compound of formula 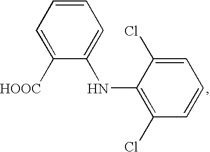
or a pharmaceutically acceptable salt thereof.

62. The process according to claim 60, wherein the compound of Formula 1d2 is a compound of formula 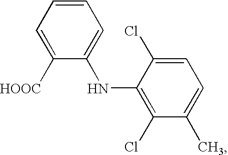
or a pharmaceutically acceptable salt thereof.

63. The process according to claim 60, wherein the compound of Formula Id2 is a compound of formula 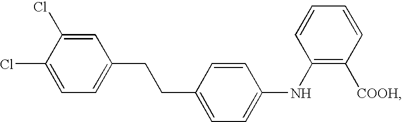
or a pharmaceutically acceptable salt thereof.

64. The process according to claim 60, wherein the compound of Formula Id2 is a compound of formula 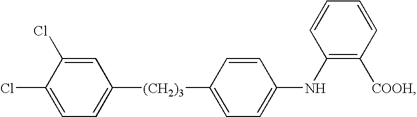
or a pharmaceutically acceptable salt thereof.

65. The process according to claim 60, wherein the compound of Formula Id2 is a compound of formula 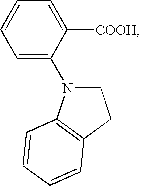
or a pharmaceutically acceptable salt thereof.

66. The process according to claim 60, wherein the compound of Formula Id2 is a compound of formula 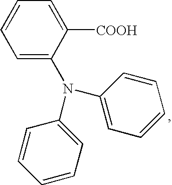
or a pharmaceutically acceptable salt thereof.

67. The process according to claim 60, wherein the compound of Formula Id2 is a compound of formula 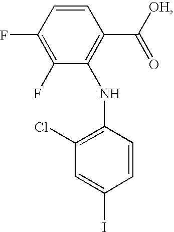
or a pharmaceutically acceptable salt thereof.

68. The process according to claim 60, wherein the compound of Formula Id2 is a compound of formula 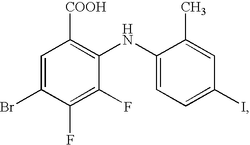
or a pharmaceutically acceptable salt thereof.

69. The process according to claim 60, wherein the compound of Formula 1d2 is a compound of formula 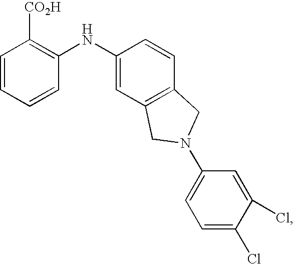
or a pharmaceutically acceptable salt thereof.

70. The process according to claim 60, wherein the compound of Formula Id2 is a compound of formula 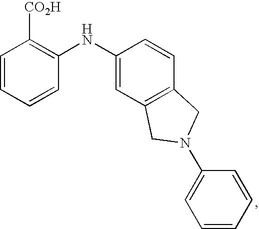
or a pharmaceutically acceptable salt thereof.

71. A process of synthesizing a compound of Formula Ie 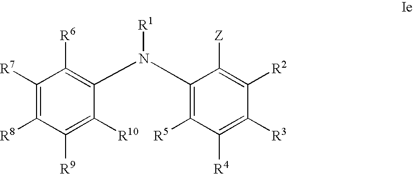
or a pharmaceutically acceptable salt thereof,
wherein:
R¹ is hydrogen, alkyl, alkoxy, or aryl;
R², R³, R⁴, R⁵, R⁶, R⁷, R⁸, R⁹, and R¹⁰ are each independently selected from:
hydrogen,
halo,
alkyl,
aryl,
a heterocyclic group,
haloalkyl,
alkoxy,
nitro,
CN,
—(O)ₘ—(CH₂)ₙ—R¹¹, or
—[N(H)]ₘ—(CH₂)ₙ—R¹¹, wherein m, n, and R¹¹ are as defined below, or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms, or
R¹ and R⁶ may be taken together with the nitrogen atom to which R¹ is attached, the carbon atom to which R⁶ is attached, and the carbon atom contiguous to said nitrogen atom to which R¹ is attached and said carbon atom to which R⁶ is attached, to form a 5-membered or 6-membered, aromatic or dihydro-aromatic ring having carbon atoms and 1 or 2 nitrogen atoms;
R¹¹ is hydrogen, hydroxy, —CO₂H, or N(R¹²)R¹³,
R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;
m is an integer of 0 or 1;
n is an integer selected from 0, 1, 2, 3, 4; and
Z is COOR¹⁵, —C(O)N(R¹⁶)R¹⁷, or —C(O)N(R¹⁸)OR¹⁹, wherein
R¹⁵ is alkyl, alkenyl, alkynyl, aryl, or a heterocyclic group, and
R¹⁶, R¹⁷, R¹⁸, and R¹⁹ are each independently selected from hydrogen, alkyl, alkenyl, phenyl, and benzyl, or
R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;
comprising coupling a compound of Formula If 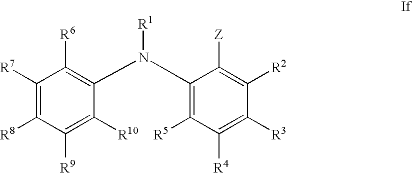
wherein Z is COOH or COOM, wherein M is a Group I metal cation or a hemi Group II metal cation, and R¹, R², R³, R⁴, R⁵, R⁶, R⁷, R⁸, R⁹, and R¹⁰ are as defined above, or when Z is COOM, R¹ is optionally a Group I metal cation or a hemi Group II metal cation,
with a compound of Formula II
HOR¹⁵ II
wherein R¹⁵ is as defined above, or a pharmaceutically acceptable salt thereof,
or with a compound of Formula III
HN(R¹⁶)R¹⁷ III
or a pharmaceutically acceptable salt thereof, wherein R¹⁶ and R¹⁷ are as defined above, or with a compound of Formula IV
HN(R¹⁸)OR¹⁹ IV
or a pharmaceutically acceptable salt thereof, wherein R¹⁸ and R¹⁹ are as defined above.

72. The process according to claim 71, wherein R¹⁸ is hydrogen and R¹⁹ is selected from methyl, ethyl, propyl, isopropyl, 1-butyl, 2-butyl, 2-methyl-prop-1-yl, 1,1-dimethylethyl, 1-buten-1-yl, 1-buten-2-yl, 1-buten-3-yl, 1-buten-4-yl, 2-buten-1-yl, 2-buten-2-yl, 1-methylcyclopropyl, 2-methylcyclopropyl, 1-methylcyclobutyl, 2-methylcyclobutyl, 3-methylcyclobutyl, 1-methylcyclopentyl, 2-methylcyclopentyl, 3-methylcyclopentyl, 1-methylcyclohexyl, 2-methylcyclohexyl, 3-methylcyclohexyl, 4-methylcyclohexyl, cyclopropylmethyl, cyclopropyl-difluoromethyl, cyclopropyl-difluoromethyl, cyclobutylmethyl, cyclopentylmethyl, cyclohexylmethyl, phenyl, and benzyl.

73. The process according to claim 72, wherein R¹⁸ is hydrogen and R¹⁹ is cyclopropylmethyl.

74. The process according claims 71 wherein the compound of Formula I is a compound of formula 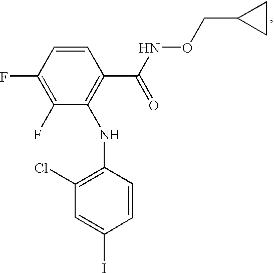
or a pharmaceutically acceptable salt thereof.

75. The process according to claim 71, wherein R¹⁶ is hydrogen and R¹⁷ is cyclopropylmethyl, 2-cyclopropylethyl, cyclobutylmethyl, 2-cyclobutylethyl, cyclopentylmethyl, 2-cyclopentylethyl, cyclohexylmethyl, 2-cyclohexylethyl, cyclopropyl-difluoromethyl, or 2-cyclopropyl-1,1-difluoroethyl.

76. The process according to claim 71 of synthesizing a compound of Formula Ig 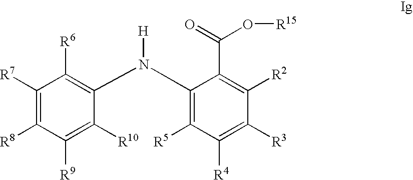
or a pharmaceutically acceptable salt thereof,
or a compound of Formula Ih 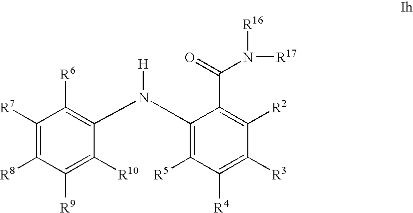
or a pharmaceutically acceptable salt thereof,
or a compound of Formula Ii 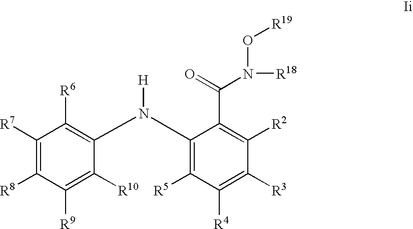
or a pharmaceutically acceptable salt thereof,
wherein:
R², R³, R⁴, R⁵, R⁶, R⁷, R⁸, R⁹, and R¹⁰ are each independently selected from:
hydrogen,
halo,
alkyl,
aryl,
a heterocyclic group,
haloalkyl,
alkoxy,
nitro,
CN,
—(O)ₘ—(CH₂)ₙ—R¹¹, or
—[N(H)]ₘ—(CH₂)ₙ—R¹¹, wherein m, n, and R¹¹ are as defined below, or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms;
R¹¹ is hydrogen, hydroxy, —CO₂H, or N(R¹²)R¹³,
R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;
m is an integer of 0 or 1;
n is an integer selected from 0, 1, 2, 3, 4; and
R¹⁵ is alkyl, alkenyl, alkynyl, aryl, or a heterocyclic group,
R¹⁶, R¹⁷, R¹⁸, and R¹⁹ are each independently selected from hydrogen, alkyl, alkenyl, phenyl, and benzyl, or
R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl comprising
(a) reacting an acid selected from trifluoroacetic acid, trichloroacetic acid, a mineral acid, an alkylsulfonic acid, and an arylsulfonic acid with a compound of Formula Ij 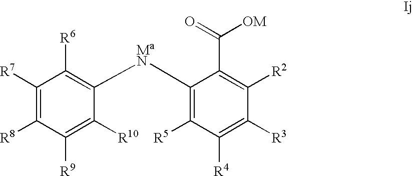
wherein R², R³, R⁴, R⁵, R⁶, R⁷, R⁸, R⁹, and R¹⁰ are as defined above,
M and M^{a} are each independently a Group I metal cation or a hemi Group II metal cation;
(b) adding a carboxylic acid activating reagent to the mixture of Step (a), and reacting for a time, and at a temperature, sufficient to form a corresponding activated carboxylic acid intermediate; and
(c) adding, optionally in the presence of up to 10 mol equivalents of a tertiary organic amine, a reactant which is selected from:
a compound of Formula II
HOR¹⁵ II
or a pharmaceutically acceptable salt thereof, wherein R¹⁵ is as defined above, or a compound of Formula III
HN(R¹⁶)R¹⁷ III
or a pharmaceutically acceptable salt thereof, wherein R¹⁶ and R¹⁷ are as defined above, or a compound of Formula IV
HN(R¹⁸)OR¹⁹ IV
or a pharmaceutically acceptable salt thereof, wherein R¹⁸ and R¹⁹ are as defined above, and reacting for a time, and at a temperature, sufficient to provide a compound of Formula Ig, Ih, or Ii, respectively.

77. The process according to claim 76, wherein M^{a} is selected from lithium cation, sodium cation, and potassium cation.

78. The process according to claim 76, wherein M^{a} is lithium cation.

79. The process according to claim 76, wherein in Step (a), the acid employed is trifluoroacetic acid, trichloroacetic acid, a mineral acid selected from HCl, HBr, or H₂SO₄, an alkylsulfonic acid selected from CH₃SO₃H and CF₃SO₃H, or an arylsulfonic acid selected from phenyl-SO₃H and para-toluenesulfonic acid.

80. The process according to claim 76, wherein in Step (a), the acid employed is CH₃SO₃H.

81. The process according to claim 76, wherein the carboxylic acid activating reagent employed in Step (b) is selected from: (COCl)₂, S(O)Cl₂, S(O)₂Cl₂, P(O)Cl₃, (phenyl)₂P(═O)Cl, 1,1′-carbonyldiimidazole, triphenylphosphine/diethylazodicarboxylate, EDC, EDCl, and N,N′-dicyclohexylcarbodiimide.

82. The process according to claim 76, wherein the carboxylic acid activating reagent employed in Step (b) is S(O)Cl₂.

83. The process according to claim 76, wherein the carboxylic acid activating reagent employed in Step (b) is (phenyl)₂P(═O)Cl.

84. The process according to claim 76, wherein the reactant added in step (c) is O-cyclopropylmethyl-hydroxylamine, or a pharmaceutically acceptable acid addition salt thereof.

85. The process according to claims 76 wherein the compound of Formula I is a compound of formula 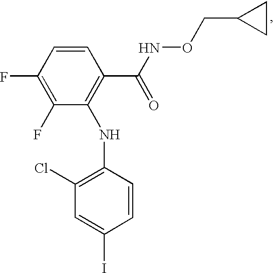
or a pharmaceutically acceptable salt thereof.

86. A process of synthesizing a compound of Formula Ik 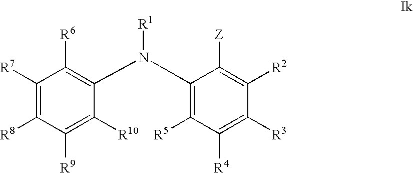
or a pharmaceutically acceptable salt thereof,
wherein:
R¹ is hydrogen, alkyl, alkoxy, or aryl;
R², R³, R⁴, R⁵, R⁶, R⁷, R⁸, R⁹, and R¹⁰ are each independently selected from:
hydrogen,
halo,
alkyl,
aryl,
a heterocyclic group,
haloalkyl,
alkoxy,
nitro,
CN,
—(O)ₘ—(CH₂)ₙ—R¹¹, or
—[N(H)]ₘ—(CH₂)ₙ—R¹¹, wherein m, n, and R¹¹ are as defined below,
or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms,
or R¹ and R⁶ may be taken together with the nitrogen atom to which R¹ is attached, the carbon atom to which R⁶ is attached, and the carbon atom contiguous to said nitrogen atom to which R¹ is attached and said carbon atom to which R⁶ is attached, to form a 5-membered or 6-membered, aromatic or dihydro-aromatic ring having carbon atoms and 1 or 2 nitrogen atoms;
R¹¹ is hydrogen, hydroxy, —CO₂H, or N(R¹²)R¹³,
R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;
m is an integer of 0 or 1;
n is an integer selected from 0, 1, 2, 3, 4; and
Z is COOR¹⁵ wherein R¹⁵ is alkyl, alkenyl, alkynyl, aryl, or a heterocyclic group;
comprising coupling a compound of Formula If 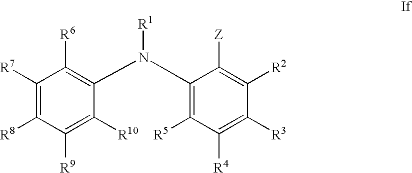
wherein Z is COOH or COOM, wherein M is a Group I metal cation or a hemi Group II metal cation, and R¹, R², R³, R⁴, R⁵, R⁶, R⁷, R⁸, R⁹, and R¹⁰ are as defined above, or when Z is COOM, R¹ is optionally a Group I metal cation or a hemi Group II metal cation,
with a compound of Formula II
HOR¹⁵ II
or a pharmaceutically acceptable salt thereof, wherein R¹⁵ is as defined above, optionally in the presence of an acid catalyst;
or with a compound of Formula IIa
L-R¹⁵ IIi
or a pharmaceutically acceptable salt thereof, wherein R¹⁵ is as defined above and L is a leaving group selected from bromo, chloro, iodo, alkylsulfonyloxy, and arylsulfonyloxy, acyloxy, optionally in the presence of a non-nucleophilic base.

87. A process for synthesizing a compound of Formula I 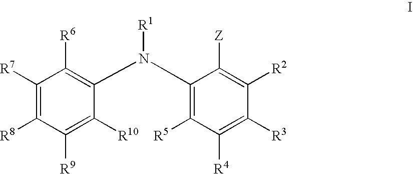
or a pharmaceutically acceptable salt thereof,
wherein:
R¹ is hydrogen, alkyl, alkoxy, or aryl;
R², R³, R⁴, R⁵, R⁶, R⁷, R⁸, R⁹, and R¹⁰ are each independently selected from:
hydrogen,
halo,
alkyl,
aryl,
a heterocyclic group,
haloalkyl,
alkoxy,
nitro,
CN,
—(O)ₘ—(CH₂)ₙ—R¹¹, or
—[N(H)]ₘ—(CH₂)ₙ—R¹¹, wherein m, n, and R¹¹ are as defined below, or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms,
or R¹ and R⁶ may be taken together with the nitrogen atom to which R¹ is attached, the carbon atom to which R⁶ is attached, and the carbon atom contiguous to said nitrogen atom to which R¹ is attached and said carbon atom to which R⁶ is attached, to form a 5-membered or 6-membered, aromatic or dihydro-aromatic ring having carbon atoms and 1 or 2 nitrogen atoms;
R¹¹ is hydrogen, hydroxy, —CO₂H, or N(R¹²)R¹³,
R¹² and R¹³ are each independently hydrogen or alkyl, or R¹² and
R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;
m is an integer of 0 or 1;
n is an integer selected from 0, 1, 2, 3, 4; and
Z is COOH, COOM, COOR15, —C(O)R¹⁵, —C(O)N(R¹⁶)R¹⁷, —C(O)N(R¹⁸)OR¹⁹, NO₂, or CN, wherein
M is a Group I metal cation or a hemi Group II metal cation,
R¹⁵ is alkyl, alkenyl, alkynyl, aryl, or a heterocyclic group, and
R¹⁶, R¹⁷, R¹⁸, and R¹⁹ are each independently selected from hydrogen, alkyl, alkenyl, phenyl, and benzyl, or
R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl,
comprising:
(a) a step for reacting a compound of Formula (A) 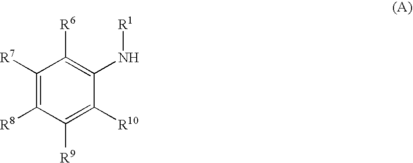
wherein R¹, R⁶, R⁷, R⁸, R⁹, and R¹⁰ are as defined above, with a compound of Formula (B) 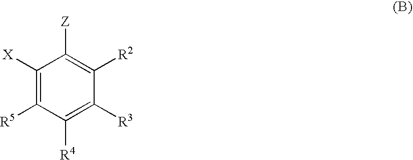
wherein Z, R², R³, R⁴, and R⁵ are as defined above, and X is halo or O-LG, wherein LG is SO₂R²⁰ or P(═O)(OR²⁰)₂, wherein R²⁰ is alkyl or aryl,
in the presence of from about 1 mol equivalent to about 10 mol equivalents of a base, wherein the base is selected from:
a Group I metal cation hydride or a Group 2 metal cation hydride, including lithium hydride, sodium hydride, potassium hydride, and calcium hydride,
a Group I metal cation dialkylamide or a Group 2 metal cation dialkylamide, including lithium diisopropylamide,
a Group I metal cation amide or a Group 2 metal cation amide, including lithium amide, sodium amide, potassium amide, and
a Group I metal cation alkoxide or a Group 2 metal cation alkoxide, including sodium ethoxide, potassium tert-butoxide, and magnesium ethoxide, for a time, and at a temperature, sufficient to yield a compound of Formula I; and
(b) purifying the compound of Formula I produced in step (a).

88. A process for synthesizing a compound of Formula I 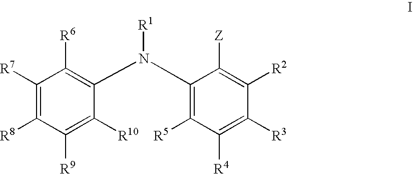
or a pharmaceutically acceptable salt thereof,
wherein:
R¹ is hydrogen, alkyl, alkoxy, or aryl;
R², R³, R⁴, R⁵, R⁶, R⁷, R⁸, R⁹, and R¹⁰ are each independently selected from:
hydrogen,
halo,
alkyl,
aryl,
a heterocyclic group,
haloalkyl,
alkoxy,
nitro,
CN,
—(O)ₘ—(CH₂)ₙ—R¹¹, or
—[N(H)]ₘ—(CH₂)ₙ—R¹¹, wherein m, n, and R¹¹ are as defined below, or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms,
or R¹ and R⁶ may be taken together with the nitrogen atom to which R¹ is attached, the carbon atom to which R⁶ is attached, and the carbon atom contiguous to said nitrogen atom to which R¹ is attached and said carbon atom to which R⁶ is attached, to form a 5-membered or 6-membered, aromatic or dihydro-aromatic ring having carbon atoms and 1 or 2 nitrogen atoms;
R¹¹ is hydrogen, hydroxy, —CO₂H, or N(R¹²)R¹³,
R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;
m is an integer of 0 or 1;
n is an integer selected from 0, 1, 2, 3, 4; and
Z is COOR¹⁵, —C(O)R¹⁵, —C(O)N(R¹⁶)R¹⁷, —C(O)N(R¹⁸)OR¹⁹, NO₂, or CN, wherein
R¹⁵ is alkyl, alkenyl, alkynyl, aryl, or a heterocyclic group, and
R¹⁶, R¹⁷, R¹⁸, and R¹⁹ are each independently selected from hydrogen, alkyl, alkenyl, phenyl, and benzyl, or
R¹⁶ and R¹⁷ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl,
comprising:
(a) a step for reacting a compound of Formula (A) 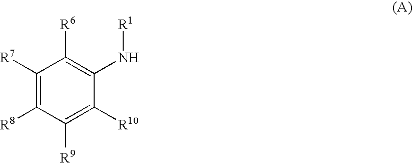
wherein R¹, R⁶, R⁷, R⁸, R⁹, and R¹⁰ are as defined above, with a compound of Formula (B) 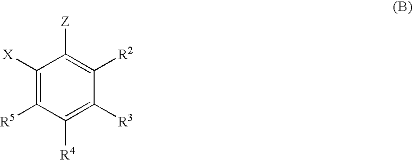
wherein Z, R², R³, R⁴, and R⁵ are as defined above, and X is halo or O-LG, wherein LG is SO₂R²⁰ or P(═O)(OR²⁰)₂, wherein R²⁰ is alkyl or aryl,
in the presence of from about 1 mol equivalent to about 10 mol equivalents of a base, wherein the base is a Group I metal cation bis(trialkylsilyl)amide or a Group 2 metal cation bis(trialkylsilyl)amide, including lithium bis(trimethylsilyl)amide, sodium bis(trimethylsilyl)amide, or potassium bis(trimethylsilyl)amide, for a time, and at a temperature, sufficient to yield a compound of Formula I; and
(b) purifying the compound of Formula I produced in step (a).

89. A process of synthesizing a compound of Formula I 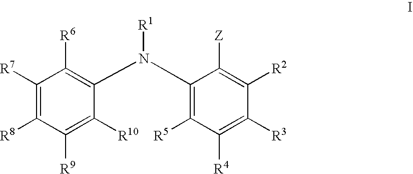
or a pharmaceutically acceptable salt thereof,
wherein:
R¹ is alkyl, alkoxy, or aryl;
R², R³, R⁴, R⁵, R⁶, R⁷, R⁸, R⁹, and R¹⁰ are each independently selected from:
hydrogen,
halo,
alkyl,
aryl,
a heterocyclic group,
haloalkyl,
alkoxy,
nitro,
CN,
—(O)ₘ—(CH₂)ₙ—R¹¹, or
—[N(H)]ₘ—(CH₂)ₙ—R¹¹, wherein m, n, and R¹¹ are as defined below,
or any two substituents selected from R², R³, R⁴, R⁵, R⁶, R⁷, R⁸, R⁹, and R¹⁰ that are bonded to contiguous ring carbon atoms, may be taken together with the contiguous ring carbon atoms themselves, to form an aryl, heteroaryl, a heterocyclic group, or cycloalkyl of from 4 to 7 total ring atoms,
or R¹ and R⁶ may be taken together with the nitrogen atom to which R¹ is attached, the carbon atom to which R⁶ is attached, and the carbon atom contiguous to said nitrogen atom to which R¹ is attached and said carbon atom to which R⁶ is attached, to form a 5-membered or 6-membered, aromatic or dihydro-aromatic ring having carbon atoms and 1 or 2 nitrogen atoms;
R¹¹ is hydrogen, hydroxy, —CO₂H, or N(R¹²)R¹³,
R¹² and R¹³ are each independently hydrogen or alkyl, or R¹² and R¹³ are taken together with the nitrogen atom to which they are attached to form a 3- to 10-membered heterocyclic group having carbon atoms and one, two, or three heteroatoms selected from O, S, and NR¹⁴, wherein R¹⁴ is hydrogen or alkyl;
m is an integer of 0 or 1;
n is an integer selected from 0, 1, 2, 3, 4; and
Z is COOH or COOM;
comprising reacting a compound of Formula (A) 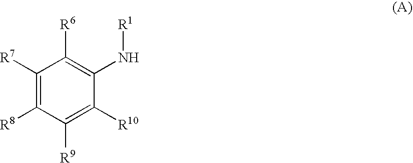
wherein R¹, R⁶, R⁷, R⁸, R⁹, and R¹⁰ are as defined above, with a compound of Formula (B) 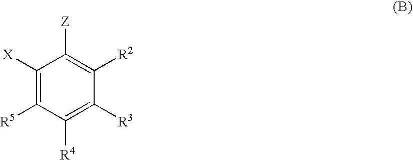
wherein Z, R², R³, R⁴, and R⁵ are as defined above, and X is halo or O-LG, wherein LG is SO₂R²⁰ or P(═O)(OR²⁰)₂, wherein R²⁰ is alkyl or aryl, optionally in a solvent, and in the presence of from about 1 mol equivalent to about 10 mol equivalents of a base, wherein the base is a Group I metal cation bis(trialkylsilyl)amide or a Group 2 metal cation bis(trialkylsilyl)amide, including lithium bis(trimethylsilyl)amide, sodium bis(trimethylsilyl)amide, or potassium bis(trimethylsilyl)amide, for a time, and at a temperature, sufficient to yield a compound of Formula I.

90. The process according to any one of claims 1, 38, 71, 86, 87, 88, or 89, wherein the process is carried out on a commercial scale.
